(19) United States
(12) Patent Application Publication
(10) Pub. No.: US 2024/0238184 A1
(43) Pub. Date: Jul. 18, 2024

(54) ANTI-INFLAMMATORY PEPTIDES, AND USES THEREOF

(71) Applicant: Nuritas Limited, South Dublin (IE)

(72) Inventors: Nora Khaldi, Dublin (IE); Cyril Lopez, Dublin (IE); Alessandro Adelfio, Dublin (IE)

(73) Assignee: Nuritas Limited, South Dublin (IE)

(21) Appl. No.: 18/451,560

(22) Filed: Aug. 17, 2023

Related U.S. Application Data
(62) Division of application No. 17/574,865, filed on Jan. 13, 2022, now Pat. No. 11,779,531.
Application No. 17/574,865 is a continuation of application No. 16/569,159, filed on Sep. 12, 2019, now Pat. No. 11,253,456.
Application No. 16/569,159 is a division of application No. 15/744,304, filed on Jan. 12, 2018, now Pat. No. 10,463,591.

(30) Foreign Application Priority Data
Jul. 16, 2015 (EP) 15177013.8
Jul. 16, 2015 (EP) 15177017.9

(51) Int. Cl. A61K 8/64; A61K 9/00; A61K 9/14; A61P 29/00; C07K 14/415; A61K 38/00

(52) U.S. Cl. CPC A61K 8/64; A61K 9/0014; A61K 9/0053; A61K 9/14; A61P 29/00; C07K 14/415; A61K 38/00

(57) ABSTRACT

An anti-inflammatory peptide comprises an anti-inflammatory fragment of a protein selected from SEQUENCE ID NOs: 1 to 16, the anti-inflammatory fragment being 7 to 37 amino acids in length and having a charge of between −9 and +3; wherein the c-terminal amino acid is not cysteine (C) or methionine (M), and the n-terminal amino acid is not cysteine (C), histidine (H), proline (P) or threonine (T). The anti-inflammatory fragment does not contain cysteine (C). The anti-inflammatory fragment is from a region of the proteins of SEQUENCE ID NOs: 1 to 16, which region is characterised by being 17 to 109 amino acids in length and having a charge of between −6 and +4, wherein the c-terminal amino acid of the region is not aspartic acid (D), phenylalanine (F), methionine (M) or tryptophan (W), and the n-terminal amino acid of the region is not aspartic acid (D), histidine (H), methionine (M), proline (P) or tryptophan (W). Examples of peptides are provided in SEQUENCE ID NOs: 71 to 221.

### CROSS REFERENCE TO RELATED APPLICATION

This application is a divisional under 35 U.S.C. § 121 of co-pending U.S. application Ser. No. 17/574,865 filed Jan. 13, 2022, which is continuation of U.S. application Ser. No. 16/569,159 filed Sep. 12, 2019, now U.S. Pat. No. 11/253,456 issued on Feb. 22, 2022, which is a divisional application of U.S. application Ser. No. 15/744,304 filed Jan. 12, 2018, now U.S. Pat. No. 10,463,591 issued on Nov. 5, 2019, which is a 35 U.S.C. § 371 National Phase Entry of the International Application No. PCT/EP2016/067090 filed Jul. 18, 2016, which designates the U.S. and which claims benefit under one or more of 35 U.S.C. § 119(a)-119(d) of European Patent Application No. 15177017.9 filed Jul. 16, 2015, and European Patent Application No. 15177013.8 filed Jul. 16, 2015, the contents of all which are incorporated herein by reference in their entirety.

### SEQUENCE LISTING

The sequence listing of the present application has been submitted electronically via Patent Center as an XML, formatted sequence listing with a file name “P11683USDIV2_SequenceListing26ST.xml”, creation date of Aug. 15, 2023 and a size of 535,867 bytes. The sequence listing submitted via Patent Center is part of the specification and is herein incorporated by reference in its entirety.

### BACKGROUND TO THE INVENTION

It is estimated that a staggering 2 billion people worldwide suffer from some type of inflammation. Inflammation is a vast biological process and an integral part of our immune response. The inflammatory response may be acute (short lived) or chronic (longer lasting) and can occur in almost every part of the body whether it be internal or external. Interestingly as well, whatever the cause of inflammation, the biological changes that occur within the body due to the inflammation response are the same throughout the body, meaning the modes of reducing inflammation, which different anti-inflammatory agents carry are the same in all parts of the body.

Typically, inflammation is a natural response and a necessary one that rids the cells of injury causes, foreign attacks, or removes dead cells. However, excess inflammation has drastic and sometimes detrimental effects on the human body. Indeed, an inflammatory response can have long-lasting, negative consequences such as tissue damage. During an inflammatory response, the body releases lysosomal enzymes which can damage tissue and can even lead to a life-threatening hypersensitivity reaction. These conditions can have long-lasting effects both externally, with the development of acute skin rashes and eczema, and internally, triggering diseases such as inflammatory bowel disease. As a result, maintaining a normal level of inflammation is very important for our health and wellbeing, both inside and out. Unfortunately, inflammation is on the rise. One of the major factors of this increase comes from our exposure to an increasing variety of external agents that our bodies are not accustomed to.

Most anti-inflammatory treatments used today are drugs. There are two major types of anti-inflammatory drugs, corticosteroids and non-steroidal anti-inflammatory drugs (NSAIDs). These include: Immunosuppressives (methotrexate, ciclosporin); Specific biological drugs (mostly TNF-alpha inhibitors, but also inhibitors of cyclooxygenase enzyme for example); Cytotoxic drugs; and Oral retinoids (acitretin). Furthermore, topical treatments exist to specifically reduce skin inflammation. Examples include creams and ointment (mostly cortisone based), and physical treatments like UV radiations. These drugs however have drastic side-effects such as gastrointestinal toxicity and anaphylactoid reactions. They can even suppress the immune system to such a point that it is made vulnerable to other diseases and pathogens.

Therefore, there is a clear need for the identification of agents having anti-inflammatory activity that are not immunosuppressive and/or cause other undesirable side effects. To that end, very specific types of food are known to reduce inflammation (Kiecolt-Glaser J. K. et al. 2010, Middleton E. et al. 2000, Chatterjee M. et al. 2005). Indeed, particular components of these specific foods are in low concentrations, hidden, or locked away, and once identified and unlocked can be scaled to specifically target inflammation in a recognized way, as our bodies understand the components of food and are able to readily process these molecules. Indeed, these particular food molecules can reduce inflammation without completely blocking this immune system response which puts the body in a vulnerable state. For those with many food allergies as well, identifying and unlocking the particular components of a food that may reduce inflammation would allow for these individuals to gain the anti-inflammatory benefits of a food they would otherwise be allergic to.

It is an object of the invention to overcome at least one of the above-referenced problems.

### SUMMARY OF THE INVENTION

The pea genome codes for over 70,000 different proteins. The Applicant has identified seven of these proteins, each of which contains one or more anti-inflammatory fragments. Likewise, out of the more than 60,000 proteins encoded by the rice genome, the Applicant has identified eight proteins, each of which contains one or more anti-inflammatory fragments. The anti-inflammatory fragments of the sixteen identified proteins have been shown to have anti-inflammatory activity when incubated with LPS-stimulated human cells (FIG. 1 to FIG. 19), do not cause human cell viability issues (FIGS. 20A and 20B) and are not toxic to human cells (FIGS. 21A and 21B). The specific plant proteins from which the natural peptides are derived are provided in SEQ ID NOs: 1-15 and 353-355, especially from regions of the proteins provided in SEQ ID NOs: 17-69. The specific pea proteins from which the peptides are derived are provided in SEQ ID NOs: NO: 1 to 5, 8 and 9, and the specific rice proteins from which the peptides are derived are provided in SEQ ID NOs: 6, 7 and 10 to 15. Homologs of these proteins are described in SEQ ID NOs: 222-267. The specific peptides initially identified in the pea proteins are shown in SEQ ID NOs: 71-107 and 110-111. The specific peptides initially identified in the rice proteins are shown in SEQ ID NOs: 108-109 and 112-220. Additional peptides of the invention identified in the pea and rice proteins are provided herein, for example in SEQ ID NOs: 320, 331 to 352, and 356-424. The peptide of the invention encompasses peptides comprising or consisting of any of the afore-mentioned peptides.

(SEQ ID NO: 331)
EWQINEK-
Fragment of rice protein of SEQ ID NO: 7
(SEQ ID NO: 332)
FLPQHTD-
Fragment of pea protein of SEQ ID NO: 1
(SEQ ID NO: 333)
GPQQYAEWQINEK-
Fragment of rice protein of SEQ ID NO: 7
(SEQ ID NO: 334)
PGQLQSFLLSGN-
Fragment of pea protein of SEQ ID NO: 1
(SEQ ID NO: 335)
PGQLQSFLLSGNQNQQNYLSGF-
Fragment of pea protein of SEQ ID NO: 1
(SEQ ID NO: 336)
PQQYAEWQ-
Fragment of rice protein of SEQ ID NO: 7
(SEQ ID NO: 337)
QLQSFLLSGNQNQQNYLSGFSK-
Fragment of pea protein of SEQ ID NO: 1
(SEQ ID NO: 338)
QNQQNYLSGFSK-
Fragment of pea protein of SEQ ID NO: 1
(SEQ ID NO: 339)
QSFLLSGNQNQQ-
Fragment of pea protein of SEQ ID NO: 1
(SEQ ID NO: 340)
QSFLLSGNQ-
Fragment of pea protein of SEQ ID NO: 1
(SEQ ID NO: 341)
RGPQQYA-
Fragment of rice protein of SEQ ID NO: 7
(SEQ ID NO: 342)
DALEPDNR-
Fragment of pea protein of SEQ ID NO: 354
(SEQ ID NO: 343)
SEEGYYGEQQQQPGMTR-
Fragment of rice protein of SEQ ID NO: 353
(SEQ ID NO: 344)
GYYGEQQQQPGMTR-
Fragment of rice protein of SEQ ID NO: 353
(SEQ ID NO: 345)
IDGYDTPVEGR-
Fragment of rice protein of SEQ ID NO: 15
(SEQ ID NO: 346)
NGVLRPGQL-
Fragment of rice protein of SEQ ID NO: 14
(SEQ ID NO: 347)
RHGEWGPSY
(SEQ ID NO: 348)
FWM-
Fragment of pea protein of SEQ ID NO: 3
[SEQ ID NO: 349]
TVFDGVLRPGQL-
Fragment of rice protein of SEQ ID NO: 10
[SEQ ID NO: 350]
RLQSQNDQRGEIIHVK-
Fragment of rice protein of SEQ ID NO: 10
[SEQ ID NO: 351]
HGPVEMPYTLLYPSSK-
Fragment of pea protein of SEQ ID NO: 355
[SEQ ID NO: 352]
LDALEPDNR-
Fragment of pea protein of SEQ ID NO: 354
[SEQ ID NO: 320]
RGPQQYAEWQINE-
Fragment of rice protein SEQ ID NO: 7

The peptides of the invention are primarily useful for causing a decrease in inflammation, and therefore have utility in the prevention or treatment of inflammatory conditions and maintaining gut health in mammals.

In a first aspect, the invention provides a peptide, typically 3 to 50 amino acids in length, and comprising a fragment of a protein disclosed herein, for example selected from SEQ ID NOs: 1 to 16, 349 or 350, or a homolog thereof, or a variant or fragment of the peptide (hereafter “peptide of the invention”). In one embodiment, the peptide or variant or fragment thereof is bioactive. In one embodiment, the peptide or variant or fragment thereof has anti-inflammatory activity.

In one embodiment, the peptide of the invention comprises a sequence selected from SEQ ID NOs: 17-220, 268-352, and 356-424.

In one embodiment, the peptide of the invention consists essentially of a sequence selected from SEQ ID NOs: 17-220, 268-352, and 356-424.

In one embodiment, the peptide consists of 3-50 amino acids. In one embodiment, the peptide consists of 4-50 amino acids. In one embodiment, the peptide consists of 5-50 amino acids. In one embodiment, the peptide consists of 6-50 amino acids.

In one embodiment, the fragment has between 7 and 37 amino acids and a charge of between −9 and +3.

Preferably, the c-terminal amino acid is not cysteine (C) or methionine (M).

Preferably, the n-terminal amino acid is not cysteine (C), histidine (H), proline (P) or threonine (T)

Preferably, the c-terminal domain of the fragment does not contain cysteine (C).

Preferably, the n-terminal domain of the fragment does not contain cysteine (C).

Preferably, the fragment does not contain cysteine (C).

Preferably, the peptide does not contain cysteine (C).

Preferably, the fragment is from a region of the proteins of SEQ ID NOs: 1 to 16, which regions are characterised by the following features:
17 to 109 amino acids in length;
a charge of between −6 and +4;
the c-terminal amino acid is not aspartic acid (D), phenylalanine (F), methionine (M) or tryptophan (W);
the n-terminal amino acid is not aspartic acid (D), histidine (H), methionine (M), proline (P) or tryptophan (W).

Preferably, the c-terminal domain of the region does not contain tryptophan (W).

Preferably, the regions of the proteins of SEQ ID NOs: 1 to 7 are selected from the SEQ ID NOs: 17-33.

Preferably, the regions of the proteins of SEQ ID NOs: 8 to 16 are selected from the SEQ ID NOs: 34-70.

Preferably, the regions of the proteins of SEQ ID NOs: 1 to 16 are selected from the SEQ ID NOs: 17-70.

Preferably, the fragment is selected from SEQ ID NOs: 71 to 221, or a variant of the fragment.

Preferably, the peptide consists of a fragment selected from SEQ ID NOs: 71 to 221, or a variant of the fragment.

Preferably, the peptide consists of a sequence selected from SEQ ID NOs: 71 to 221.

In one embodiment, the peptide comprises a fragment of a pea protein, wherein the fragment is selected from SEQ ID NOs: 71-107, 332, 334, 335, 337-340, 342, 348 and 351-352.

In one embodiment, the peptide comprises a fragment of a pea protein, wherein the fragment is selected from SEQ ID NOs: 71-107, 332, 334, 335, 337-340, 342, 348 and 351-352. In one embodiment, the fragment is selected from 339, 352, 351, 93, 92, 75, 76, 105.

In one embodiment, the peptide comprises a fragment of a rice protein, wherein the fragment is selected from SEQ ID NOs: 108-109, 112-220, 320, 331, 333, 336, 341, 343-346, 349-350. In one embodiment, the fragment is selected from 341, 144, 320, 349, 350, 177, 343-346.

In one embodiment, the peptide is a modified peptide. In one embodiment the peptide is modified with a protecting group. In one embodiment, the peptide is modified to increase its lipophilicity. In none embodiment, the peptide is modified to increase its half-life. In one embodiment, an N or C-terminal amino acid of the peptide is modified. In one embodiment, the N or C-terminal amino acid of the peptide is modified with a protecting group.

The invention also provides a conjugate comprising a peptide of the invention conjugated to a binding partner. In one embodiment, the peptide of the invention is modified with a reactive group configured to allow conjugation to the binding partner.

### SEQ ID NO: 1 (Pea Protein 1)

Preferably, the peptide comprises a bioactive fragment of the protein of SEQ ID NO: 1, or a homolog thereof, or a bioactive variant of the protein fragment.

Preferably, the peptide comprises a bioactive fragment of one of seven regions of SEQ ID NO: 1, namely SEQ ID NOS: 17 to 23, or a bioactive variant of the protein fragment.

Preferably, the peptide comprises a bioactive fragment selected from SEQ ID NOS: 71 to 91 or 360, or a bioactive variant of the protein fragment.

Preferably, the peptide comprises a fragment of the region of SEQ ID NO:17, for example SEQ ID NO: 71, or a bioactive variant of the protein fragment.

Preferably, the peptide comprises a bioactive fragment of the region of SEQ ID NO:18, for example SEQ ID NO: 72, or a bioactive variant of the fragment.

Preferably, the peptide comprises a bioactive fragment of the region of SEQ ID NO:19, for example either or both of SEQ ID NO: 73 or 74, or a bioactive variant of the fragment.

Preferably, the peptide comprises a bioactive fragment of the region of SEQ ID NO:20, for example either or both of SEQ ID NO: 75 or 76. or a bioactive variant of the fragment.

Preferably, the peptide comprises a bioactive fragment of the region of SEQ ID NO:21, for example one or more or all of SEQ ID NO: 77 to 84, or a bioactive variant of the fragment.

Preferably, the peptide comprises a bioactive fragment of the region of SEQ ID NO:22, for example one or more or all of SEQ ID NO: 85 to 89, or a bioactive variant of the fragment.

Preferably, the peptide comprises a bioactive fragment of the region of SEQ ID NO:23, for example SEQ ID NO: 90 or a bioactive variant of the fragment.

Preferably, the peptide comprises SEQ ID NO: 91, or a bioactive variant of the fragment.

The invention also provides a composition comprising at least one peptide of the invention, which peptide includes a fragment of SEQ ID NO: 1 or a homolog thereof. The invention also provides a composition comprising at least 2, preferably 3, preferably 4, preferably 5, preferably 6, preferably 7, preferably 8, preferably 9, or preferably 10 peptides of the invention, each of which comprises a different fragment of SEQ ID NO: 1 or a homolog thereof. Preferably, the peptide or peptides are bioactive. Preferably, the peptide or peptides are anti-inflammatory. Preferably, the composition comprises a first peptide of the invention that comprises afragment of a first region selected from SEQ-ID NOS: 17 to 23, and a second peptide of the invention that comprises fragment of a second region selected from SEQ ID NOS: 17 to 23. Preferably, the composition comprises a first bioactive peptide comprising a first bioactive fragment selected from SEQ ID NOs: 71 to 91 (or a bioactive variant of the fragment), and a second bioactive peptide comprising a second bioactive fragment selected from SEQ ID NOs: 71 to 91 (or a bioactive variant of the fragment).

Homologs of Pea Protein 1 (SEQ ID NO: 1) include Vicia fabia, Cicer arietinum and Lens culinaris homologs (SEQ ID NO: 222-224).

### SEQ ID NO: 2 (Pea Protein 2)

Preferably, the peptide comprises a bioactive fragment of the protein of SEQ ID NO: 2, or a homolog thereof.

Preferably, the peptide comprises a bioactive fragment of one of two regions of SEQ ID NO: 2, namely SEQ ID NOS: 24 or 25.

Preferably, the peptide comprises a bioactive fragment of the region of SEQ ID NO:24, for example SEQ ID NO: 92, or a bioactive variant of the fragment.

Preferably, the peptide comprises a bioactive fragment of the region of SEQ ID NO:25, for example SEQ ID NO: 93, or a bioactive variant of the fragment.

The invention also provides a composition comprising at least one bioactive peptide of the invention, which peptide includes a bioactive fragment of SEQ ID NO: 2 or a homolog thereof. The invention also provides a composition comprising at least two bioactive peptides of the invention, each of which comprises a different bioactive fragment of SEQ ID NO: 2 or a homolog thereof. Preferably, the composition comprises a first bioactive peptide comprising a bioactive fragment of the region of SEQ ID NO: 24, and a second bioactive peptide comprising a bioactive fragment of the region of SEQ ID NO: 25. Preferably, the composition comprises a first bioactive peptide comprising a bioactive fragment of SEQ ID NO: 92, and a second bioactive peptide comprising a bioactive fragment of SEQ ID NO: 93.

Homologs of Pea Protein 2 (SEQ ID NO: 2) include Lens culinaris, Vicia narbonensis and Glycine max (SEQ ID NOs: 225-227).

### SEQ-ID NO: 3 (Pea Protein 3)

Preferably, the peptide comprises a bioactive fragment of the protein of SEQ ID NO: 3, or a homolog thereof.

Preferably, the peptide comprises a bioactive fragment of one of two regions of SEQ ID NO: 3, namely SEQ ID NOS: 26 or 27.

Preferably, the peptide comprises a bioactive fragment of the region of SEQ ID NO:26, for example SEQ ID NO: 94, or a bioactive variant the fragment.

Preferably, the peptide comprises a bioactive fragment of the region of SEQ ID NO:27, for example SEQ ID NO: 95, or a bioactive variant of the fragment.

The invention also provides a composition comprising at least bioactive peptides of the invention, which peptide includes a bioactive fragment of SEQ ID NO: 3 or a homolog thereof. The invention also provides a composition comprising at least two bioactive peptides of the invention, each of which comprises a different bioactive fragment of SEQ ID NO: 3 or a homolog thereof. Preferably, the composition comprises a first peptide comprising a bioactive fragment of the region of SEQ ID NO: 26, and a second peptide comprising a bioactive fragment of SEQ ID NO: 27. Preferably, the composition comprises a first bioactive peptide comprising a bioactive fragment of SEQ ID NO: 94 (or a bioactive variant of the fragment), and a second bioactive peptide comprising a bioactive fragment of SEQ ID NO: 95 (or a bioactive variant of the fragment).

Homologs of Pea Protein 3 (SEQ ID NO: 3) include Vicia sativa, Medicago truncatula, and Lotus japonicas (SEQ ID NO: 228-230).

### SEQ ID NO: 4 (Pea Protein 4)

Preferably, the peptide comprises a bioactive fragment of the protein of SEQ ID NO: 4, or a homolog thereof.

Preferably, the peptide comprises a bioactive fragment of one of five regions of SEQ_ID NO: 4, namely SEQ ID NOS: 28 to 32. Preferably, the region is SEQ ID NO: 28, preferably SEQ ID NO: 29, preferably SEQ ID NO: 30, preferably SEQ ID NO: 31, preferably SEQ ID NO: 32.

Preferably, the peptide comprises a bioactive fragment of the region of SEQ ID NO:28, for example SEQ ID NO: 96, or a bioactive variant of the fragment.

Preferably, the peptide comprises a bioactive fragment of the region of SEQ ID NO:29, for example SEQ ID NO: 97 to 103, or a bioactive variant of the fragment.

Preferably, the peptide comprises a bioactive fragment of the region of SEQ ID NO:30, for example SEQ ID NO: 104, or a bioactive variant of the fragment.

Preferably, the peptide comprises a bioactive fragment of the region of SEQ ID NO:31, for example SEQ ID NO: 105, or a bioactive variant of the fragment.

Preferably, the peptide comprises a bioactive fragment of the region of SEQ ID NO:32, for example SEQ ID NO: 106, or a bioactive variant of the fragment.

The invention also provides a composition comprising at least one bioactive peptide of the invention, which peptide includes a bioactive fragment of SEQ ID NO: 4 or a homolog thereof. The invention also provides a composition comprising at least 2, preferably 3, preferably 4, preferably 5, preferably 6, preferably 7, preferably 8, preferably 9, or preferably 10 peptides of the invention that comprise different bioactive fragments of SEQ ID NO: 4 or a homolog thereof. Preferably, the composition comprises a first peptide comprising a bioactive fragment of a first region selected from SEQ ID NOS: 28 to 32, and a second peptide that comprises a bioactive fragment of a second region selected from SEQ ID NOS: 28 to 32. Preferably, the composition comprises a first bioactive peptide comprising a first bioactive fragment selected from SEQ ID NO: 96 to 106 (or a bioactive variant of the fragment), and a second bioactive peptide comprising a second bioactive fragment selected from SEQ ID NO: 96 to 106 (or a bioactive variant of the fragment).

Homologs of Pea Protein 4 (SEQ ID NO: 4) include Pisum abyssinicum, Lathyrus annuus, and Vicia villosa (SEQ ID NOs: 231-233).

### SEQ ID NO: 5 (Pea Protein 5)

Preferably, the peptide comprises a bioactive fragment of the protein of SEQ ID NO: 5, or a homolog thereof.

Preferably, the peptide comprises a bioactive fragment of a region of SEQ ID NO: 5, namely the region of SEQ ID NO: 33, for example SEQ ID NO: 107, or a bioactive variant of the fragment.

The invention also provides a composition comprising at least one bioactive peptides of the invention, which peptide includes a bioactive fragment of SEQ ID NO: 5 or a homolog thereof. The invention also provides a composition comprising at least two peptides of the invention that comprise at least one bioactive fragment of SEQ ID NO: 5 or a homolog thereof, for example SEQ ID NO: 107.

Homologs of Pea Protein 5 (SEQ ID NO: 5) include Medicago truncatula, Vicia peregrine, and Vicia lutea (SEQ ID NOs: 234-236).

### SEQ ID NO: 6 (Rice Protein 7—Q6K508)

Preferably, the peptide comprises a bioactive fragment of the protein of SEQ ID NO: 6 or a homolog thereof.

Preferably, the peptide comprises a bioactive fragment of SEQ ID NO: 108, or a bioactive variant of the fragment.

The invention also provides a composition comprising at least one bioactive peptides of the invention, which peptide includes a bioactive fragment of SEQ ID NO: 6 or a homolog thereof. The invention also provides a composition comprising at least two peptides of the invention including at least one bioactive fragment of SEQ ID NO: 6 or a homolog thereof, for example SEQ ID NO: 108 (SP1).

Homologs of Rice Protein 7 (SEQ ID NO: 6) include Oryza, brachyantha, Avena sativa, and Brachypodium distachyon (SEQ ID NOs: 237-239).

### SEQ ID NO: 7 (Rice Protein 8—Q6K7K6)

Preferably, the peptide comprises a bioactive fragment of the protein of SEQ ID NO: 7 or a homolog thereof.

Preferably, the peptide comprises a bioactive fragment of SEQ ID NO: 109 (SP2), or a bioactive variant of the fragment.

The invention also provides a composition comprising at least one bioactive peptides of the invention, which peptide includes a bioactive fragment of SEQ ID NO: 7 or a homolog thereof. The invention also provides a composition comprising at least two peptides of the invention including at least one bioactive fragment of SEQ ID NO: 7 or a homolog thereof, for example SEQ ID NO: 109 (SP2).

Homologs of Rice Protein 8 (SEQ ID NO: 7) include Oryza sativa Japonica Group, Oryza sativa Indica Group, and Oryza brachyantha (SEQ ID NO: 240-242).

### SEQ ID NO: 8 (Pea Protein 6—P13919)

Preferably, the peptide comprises a bioactive fragment of the protein of SEQ ID NO: 8 or a homolog thereof.

Preferably, the peptide comprises a bioactive fragment of SEQ ID NO: 110 (SP3), or a bioactive variant of the fragment.

The invention also provides a composition comprising at least one bioactive peptide of the invention, which peptide includes a bioactive fragment of SEQ ID NO: 8 or a homolog thereof. The invention also provides a composition comprising at least two peptides of the invention including at least one bioactive fragment of SEQ ID NO: 8 or a homolog thereof, for example SEQ ID NO: 110 (SP3).

Homologs of Pea Protein 8 (SEQ ID NO: 8) include Pisum fulvum, Pisum abyssinicum and Vicia villosa (SEQ ID NO: 243-245).

### SEQ ID NO: 9 (Pea Protein 7—P02855)

Preferably, the peptide comprises a bioactive fragment of the protein of SEQ ID NO: 9 or a homolog thereof.

Preferably, the peptide comprises a bioactive fragment of SEQ ID NO: 111 (SP4), or a bioactive variant of the fragment.

The invention also provides a composition comprising at least one bioactive peptide of the invention, which peptide includes a bioactive fragment of SEQ ID NO: 9 or a homolog thereof. The invention also provides a composition comprising at least two peptides of the invention including at least one bioactive fragment of SEQ ID NO: 9 or a homolog thereof, for example SEQ ID NO: 111 (SP4).

Homologs of Pea Protein 9 (SEQ ID NO: 9) include Lathyrus hirsutus, Lathyrus cicero, Lathyrus sativus (SEQ ID NO: 246-248).

### SEQ ID NO: 10 (Rice Protein 1—P07728)

Preferably, the peptide comprises a bioactive fragment of the protein of SEQ ID NO: 10 or a homolog thereof.

Preferably, the peptide comprises a fragment of one of nine regions of SEQ ID NO: 10, namely SEQ ID NOS: 34 to 42. Preferably, the region is SEQ ID NO: 34, preferably SEQ ID NO: 35, preferably SEQ ID NO: 36, preferably SEQ ID NO: 37, preferably SEQ ID NO: 38, preferably SEQ ID NO: 39, preferably SEQ ID NO: 40, preferably SEQ ID NO: 41, preferably SEQ ID NO: 42.

Preferably, the peptide comprises a bioactive fragment of the region of SEQ ID NO:34, for example SEQ ID NO: 112 or SEQ ID NO: 113 or a bioactive variant of the fragment.

Preferably, the peptide comprises a bioactive fragment of the region of SEQ ID NO:35, for example SEQ ID NO: 114, or a bioactive variant of the fragment.

Preferably, the peptide comprises a bioactive fragment of the region of SEQ ID NO:36, for example SEQ ID NO: 115 or SEQ ID NO: 116, or a bioactive variant of the fragment.

Preferably, the peptide comprises a bioactive fragment of the region of SEQ ID NO:37, for example SEQ ID NO: 117 or SEQ ID NO: 118, or a bioactive variant of the fragment.

Preferably, the peptide comprises a bioactive fragment of the region of SEQ ID NO:38, for example SEQ ID NO: 119, or a bioactive variant of the fragment.

Preferably, the peptide comprises a bioactive fragment of the region of SEQ ID NO:39, for example SEQ ID NOs: 120, 121 or 122, or a bioactive variant of the fragment.

Preferably, the peptide comprises a bioactive fragment of the region of SEQ ID NO:40, for example SEQ ID NO: 123, or a bioactive variant of the fragment.

Preferably, the peptide comprises a bioactive fragment of the region of SEQ ID NO:41, for example SEQ ID NOs: 124, 125, 126, 127, 128 or 129, or a bioactive variant of the fragment.

Preferably, the peptide comprises a fragment of the region of SEQ ID NO:42, for example SEQ ID NOs: 130, 131 or 132, or a bioactive variant of the fragment.

The invention also provides a composition comprising at least one bioactive peptide of the invention, which peptide includes a bioactive fragment of SEQ ID NO: 10 or a homolog thereof. The invention also provides a composition comprising at least 2, preferably 3, preferably 4, preferably 5, preferably 6, preferably 7, preferably 8, preferably 9, or preferably 10 peptides of the invention, each of which comprises a different bioactive fragment of SEQ ID NO: 10 or a homolog thereof. Preferably, the composition comprises a first peptide that comprises a bioactive fragment of a first region selected from SEQ ID NOS: 34 to 42, and a second peptide that comprises a bioactive fragment of a second region selected from SEQ ID NOS: 34 to 42. Preferably, the composition comprises a first bioactive peptide comprising a first bioactive fragment selected from SEQ ID NOs: 112 to 132 (or a bioactive variant of the fragment), and a second bioactive peptide comprising a second bioactive fragment selected from SEQ ID NO: 112 to 132 (or a bioactive variant of the fragment).

Homologs of Rice Protein 1 (SEQ ID NO: 10) include Oryza brachyantha, and Zizania latifolia (SEQ ID NO: 249-251).

### SEQ ID NO: 11 (Rice Protein 2—P07728)

Preferably, the peptide comprises a bioactive fragment of the protein of SEQ ID NO: 11 or a homolog thereof.

Preferably, the peptide comprises a bioactive fragment of one of four regions of SEQ ID NO: 11, namely SEQ ID NOS: 43 to 46. Preferably, the region is SEQ ID NO: 43, preferably SEQ ID NO: 44, preferably SEQ ID NO: 45, preferably SEQ ID NO: 46.

Preferably, the peptide comprises a bioactive fragment of the region of SEQ ID NO:43, for example SEQ ID NO: 133 or a bioactive variant of the fragment.

Preferably, the peptide comprises a bioactive fragment of the region of SEQ ID NO:44, for example SEQ ID NO: 134 to 137, or a bioactive variant of the fragment.

Preferably, the peptide comprises a bioactive fragment of the region of SEQ ID NO:45, for example SEQ ID NO: 138 to 144, or a bioactive variant of the fragment.

Preferably, the peptide comprises a bioactive fragment of the region of SEQ ID NO:46, for example SEQ ID NO:145, or a bioactive variant of the fragment.

The invention also provides a composition comprising at least one bioactive peptides of the invention, which peptide includes a bioactive fragment of SEQ ID NO:11 or a homolog thereof. The invention also provides a composition comprising at least 2, preferably 3, preferably 4, preferably 5, preferably 6, preferably 7, preferably 8, preferably 9, or preferably 10 peptides of the invention, each of which comprises a bioactive fragment of SEQ ID NO: 11 or a homolog thereof. Preferably, the composition comprises a first peptide that comprises a bioactive fragment of a first region selected from SEQ ID NOS: 43 to 46, and a second peptide that comprises a bioactive fragment of a second region selected from SEQ ID NOS: 43 to 46. Preferably, the composition comprises a first bioactive peptide comprising a first bioactive fragment selected from SEQ ID NOs: 133 to 145 (or a bioactive variant of the fragment), and a second bioactive peptide comprising a second bioactive fragment selected from SEQ ID NOs: 133 to 145 (or a bioactive variant of the fragment).

Homologs of Rice Protein 2 (SEQ ID NO: 11) include Oryza sativa Indica Group, Zizania latifolia, Avena sativa (SEQ ID NOs: 252-254).

### SEQ ID NO: 12 (Rice Protein 3—P07730)

Preferably, the peptide comprises a bioactive fragment of the protein of SEQ ID NO: 12 or a homolog thereof.

Preferably, the peptide comprises a bioactive fragment of one of eight regions of SEQ ID NO: 12, namely SEQ ID NOS: 47 to 54. Preferably, the region is SEQ ID NO: 47, preferably SEQ ID NO: 48, preferably SEQ ID NO: 49, preferably SEQ ID NO: 50, preferably SEQ ID NO: 51, preferably SEQ ID NO: 52, preferably SEQ ID NO: 53, preferably SEQ ID NO: 54.

Preferably, the peptide comprises a bioactive fragment of the region of SEQ ID NO:47, for example one of SEQ ID NOs: 146 to 150, or a bioactive variant of the fragment.

Preferably, the peptide comprises a bioactive fragment of the region of SEQ ID NO:48, for example one of SEQ ID NOs: 151 to 157, or a bioactive variant of the fragment.

Preferably, the peptide comprises a bioactive fragment of the region of SEQ ID NO:49, for example one of SEQ ID NOs: 158 or SEQ ID NO: 159, or a bioactive variant of the fragment.

Preferably, the peptide comprises a bioactive fragment of the region of SEQ ID NO:50, for example one of SEQ ID NOs: 160 to 162, or a bioactive variant of the fragment.

Preferably, the peptide comprises a bioactive fragment of the region of SEQ ID NO:51, for example one of SEQ ID NOs: 163 or 164, or a bioactive variant of the fragment.

Preferably, the peptide comprises a bioactive fragment of the region of SEQ ID NO:52, for example SEQ ID NO: 165, or a bioactive variant of the fragment.

Preferably, the peptide comprises a bioactive fragment of the region of SEQ ID NO:53, for example one of SEQ ID NO: 166 to 171, or a bioactive variant of the fragment.

Preferably, the peptide comprises a bioactive fragment of the region of SEQ ID NO:54, for example one of SEQ ID NO: 172 to 176, or a bioactive variant of the fragment.

The invention also provides a composition comprising at least one bioactive peptides of the invention, which peptide includes a bioactive fragment of SEQ ID NO: 12 or a homolog thereof. The invention also provides a composition comprising at least 2, preferably 3, preferably 4, preferably 5, preferably 6, preferably 7, preferably 8, preferably 9, or preferably 10 peptides of the invention, each of which comprises a bioactive fragment of SEQ ID NO: 12 or a homolog thereof. Preferably, the composition comprises a first peptide that comprises a bioactive fragment of a first region selected from SEQ ID NOS: 47 to 54, and a second peptide that comprises a bioactive fragment of a second region selected from SEQ ID NOS: 47 to 54. Preferably, the composition comprises a first bioactive peptide comprising a first bioactive fragment selected from SEQ ID NOs: 146 to 172 (or a bioactive variant of the fragment), and a second bioactive peptide comprising a second anti-inflammatory fragment selected from SEQ ID NOs: 146 to 172 (or a bioactive variant of the fragment).

Homologs of Rice Protein 3 (SEQ ID NO: 12) include Oryza brachyantha, Brachipodium distachyon (SEQ ID NOs: 255-257).

### SEQ ID NO: 13 (Rice Protein 4—Q0D7S0)

Preferably, the peptide comprises a bioactive fragment of the protein of SEQ ID NO: 13 or a homolog thereof.

Preferably, the peptide comprises a bioactive fragment of a region of SEQ ID NO: 13, namely SEQ ID NO: 55, for example SEQ ID NO: 177, or a bioactive variant of the fragment.

The invention also provides a composition comprising at least one bioactive peptide of the invention, which peptide includes a bioactive fragment of SEQ ID NO: 13 or a homolog thereof. The invention also provides a composition comprising at least 2 peptides of the invention, at least one of which comprises a bioactive fragment of SEQ ID NO: 13 or a homolog thereof, for example SEQ ID NO: 177, or a bioactive variant of the fragment.

Homologs of Rice Protein 4 (SEQ ID NO: 13) include Oryza sativa Indica Group, Zizania latifolia, Avena sativa (SEQ ID NOs: 252-254).

### SEQ ID NO: 14 (Rice Protein 5—P14614)

Preferably, the peptide comprises a fragment of the protein of SEQ ID NO: 14 or a homolog thereof.

Preferably, the peptide comprises a bioactive fragment of one of seven regions of SEQ ID NO: 14, namely SEQ ID NOS: 56 to 62. Preferably, the region is SEQ ID NO: 56, preferably

SEQ ID NO: 57, preferably SEQ ID NO: 58, preferably SEQ ID NO: 59, preferably SEQ ID NO: 60, preferably SEQ ID NO: 61, preferably SEQ ID NO: 62.

Preferably, the peptide comprises a bioactive fragment of the region of SEQ ID NO:56, for example one of SEQ ID NOs: 178 to 180 or a bioactive variant of the fragment.

Preferably, the peptide comprises a bioactive fragment of the region of SEQ ID NO:57, for example one of SEQ ID NOs: 181 to 182, or a bioactive variant of the fragment.

Preferably, the peptide comprises a bioactive fragment of the region of SEQ ID NO:58, for example SEQ ID NO: 183, or a bioactive variant of the fragment.

Preferably, the peptide comprises a bioactive fragment of the region of SEQ ID NO:59, for example one of SEQ ID NOs: 184 to 190, or a bioactive variant of the fragment.

Preferably, the peptide comprises a bioactive fragment of the region of SEQ ID NO:60, for example SEQ ID NO:191, or a bioactive variant of the fragment.

Preferably, the peptide comprises a bioactive fragment of the region of SEQ ID NO:61, for example one of SEQ ID NOs: 192 to 195, or a bioactive variant of the fragment.

Preferably, the peptide comprises a bioactive fragment of the region of SEQ ID NO:62, for example one of SEQ ID NOs: 196 to 197, or a bioactive variant of the fragment.

The invention also provides a composition comprising at least one bioactive peptides of the invention, which peptide includes a bioactive fragment of SEQ ID NO: 14 or a homolog thereof. The invention also provides a composition comprising at least 2, preferably 3, preferably 4, preferably 5, preferably 6, preferably 7, preferably 8, preferably 9, or preferably 10 peptides of the invention, each of which comprises a bioactive fragment of SEQ ID NO: 14 or a homolog thereof. Preferably, the composition comprises a first peptide that comprises a bioactive fragment of a first region selected from SEQ ID NOS: 56 to 62, and a second peptide that comprises a bioactive fragment of a second region selected from SEQ ID NOS: 56 to 62. Preferably, the composition comprises a first bioactive peptide comprising a first bioactive fragment selected from SEQ ID NOs: 178 to 197 (or a bioactive variant of the fragment), and a second bioactive peptide comprising a second bioactive fragment selected from SEQ ID NOs: 178 to 197 (or a bioactive variant of the fragment).

Homologs of Rice Protein 5 (SEQ ID NO: 14) include Oryza sativa Japonica Group, Brachipodium distachyon (SEQ ID NOs: 261-263).

### SEQ ID NO: 15 (Rice Protein 6—Q0DEV5)

Preferably, the peptide comprises a bioactive fragment of the protein of SEQ ID NO: 15 or a homolog thereof.

Preferably, the peptide comprises a bioactive fragment of one of seven regions of SEQ ID NO: 15, namely SEQ ID NOS: 63 to 67. Preferably, the region is SEQ ID NO: 63, preferably SEQ ID NO: 64, preferably SEQ ID NO: 65, preferably SEQ ID NO: 66, preferably SEQ ID NO: 67.

Preferably, the peptide comprises a fragment of the region of SEQ ID NO:63, for example one of SEQ ID NOs: 198 to 200, or a bioactive variant of the fragment.

Preferably, the peptide comprises a fragment of the region of SEQ ID NO:64, for example one of SEQ ID NO: 201 to 203 or a bioactive variant of the fragment.

Preferably, the peptide comprises a fragment of the region of SEQ ID NO:65, for example SEQ ID NO: 204 or a bioactive variant of the fragment.

Preferably, the peptide comprises a fragment of the region of SEQ ID NO:66, for example one of SEQ ID NOs: 205 to 208 or a bioactive variant of the fragment.

Preferably, the peptide comprises a fragment of the region of SEQ ID NO:67, for example one of SEQ ID NOs:209 to 215 or a bioactive variant of the fragment.

Preferably, the peptide comprises a fragment of the region of SEQ ID NO:68, for example one of SEQ ID NOs: 216 to 219, or a bioactive variant of the fragment.

Preferably, the peptide comprises a fragment of the region of SEQ ID NO:69, for example SEQ ID NO: 220, or a bioactive variant of the fragment.

The invention also provides a composition comprising at least one bioactive peptide of the invention, which peptide includes a bioactive fragment of SEQ ID NO: 15 or a homolog thereof. The invention also provides a composition comprising at least 2, preferably 3, preferably 4, preferably 5, preferably 6, preferably 7, preferably 8, preferably 9, or preferably 10 peptides of the invention, each of which comprises a bioactive fragment of SEQ ID NO: 15 or a homolog thereof. Preferably, the composition comprises a first peptide that comprises a bioactive fragment of a first region selected from SEQ ID NOS: 63 to 69, and a second peptide that comprises a bioactive fragment of a second region selected from SEQ ID NOS: 63 to 69. Preferably, the composition comprises a first bioactive peptide comprising a first bioactive fragment selected from SEQ ID NOs: 198 to 220 (or a bioactive variant of the fragment), and a second bioactive peptide comprising a second bioactive fragment selected from SEQ ID NOs: 198 to 220 (or a bioactive variant of the fragment).

Homologs of Rice Protein 6 (SEQ ID NO: 15) include Oryza rufipogon, Oryza officinalis, Hordeum vulgare subsp. vulgare (SEQ ID NOs: 264-266).

### SEQ ID NO: 16 (Bacterial Protein 1—P0C1U8)

Preferably, the peptide comprises a bioactive fragment of the protein of SEQ ID NO: 16 or a homolog thereof.

Preferably, the peptide comprises a bioactive fragment of a region of SEQ ID NO: 16, namely the region of SEQ ID NO: 70. Typically, the peptide is SEQ ID NO: 221, or a bioactive variant of the fragment.

The invention also provides a composition comprising at least one bioactive peptides of the invention, which peptide includes a bioactive fragment of SEQ ID NO: 16 or a homolog thereof. The invention also provides a composition comprising at least 2 peptides of the invention, at least one of which comprises a fragment of SEQ ID NO: 16 or a homolog thereof, for example SEQ ID NO: 212 or a bioactive variant of the fragment.

Homologs of Bacterial Protein 1 (SEQ ID NO: 16) include:
>gi|580560623|gb|EVF84961.1|glutamyl endopeptidase [Staphylococcus aureus COAS6020]
>gi|580687002|gb|EVH10169.1|glutamyl endopeptidase [Staphylococcus aureus UCIM6080]
>gi|751815683|gb|KIN24957.1|glutamyl endopeptidase [Staphylococcus aureus MRSA_CVM43477]
>gi|781884797|dbj|BAR08486.1|glutamyl endopeptidase precursor [Staphylococcus aureus subsp. aureus]
>gi|781887762|dbj|BAR11210.1|glutamyl endopeptidase precursor [Staphylococcus aureus subsp. aureus]

The invention also provides bioactive composition comprising at least one and preferably a plurality of bioactive peptides of the invention, wherein each of the peptides of the invention comprises a bioactive fragment of a protein described herein. In one embodiment the protein is selected from SEQ ID NOs: 1 to 16. Typically, each peptide of the invention is selected from, or comprises a bioactive fragment selected from, SEQ ID NOs: 71-220, or a bioactive variant of the fragment.

In one embodiment, the composition comprises a peptide of the invention, which peptide comprises a sequence selected from SEQ ID NOs: 17-220, 268-352, and 356-424.

In one embodiment, the composition comprises peptide of the invention, which peptide consists essentially of a sequence selected from SEQ ID NOs: 17-220, 268-352, and 356-424.

Typically, each peptide of the invention is selected from, or comprises a bioactive fragment selected from, SEQ ID NOs: 71-107 and 110-111, or a bioactive variant of the fragment.

Typically, each peptide of the invention is selected from, or comprises an anti-inflammatory fragment selected from, SEQ ID NOs: 108-109 and 112 to 220, or an anti-inflammatory variant of the fragment.

In embodiment, the bioactive peptide, variant or fragment of the invention is anti-inflammatory.

Preferably, the composition comprises at least two distinct peptides of the invention.

Preferably, the composition comprises at least three distinct peptides of the invention.

Preferably, the composition comprises at least four distinct peptides of the invention.

Preferably, the composition comprises at least five distinct peptides of the invention.

Preferably, the composition comprises at least six distinct peptides of the invention.

Preferably, the composition comprises at least seven distinct peptides of the invention.

Preferably, the composition comprises at least eight distinct peptides of the invention.

Preferably, the composition comprises at least nine distinct peptides of the invention.

Preferably, the composition comprises at least ten distinct peptides of the invention.

In one embodiment, the invention comprises a composition comprises one or more of SEQ ID NOs: 71-107 and 110-111, or anti-inflammatory variants of the fragments, or a mixture of the anti-inflammatory fragments and variants.

In one embodiment, the composition comprises at least one, two, three, four, five or all of SEQ ID NOS: 75, 91, 92, 93, 110 and 111.

In one embodiment, the composition comprises substantially all of fragments SEQ ID NOs: 71-107 and 110-111.

In one embodiment, the invention comprises a composition comprises one or more of SEQ ID NOs: 108-109 and 112 to 220, or anti-inflammatory variants of the fragments, or a mixture of the anti-inflammatory fragments and variants.

In one embodiment, the composition comprises at least one, two or three of SEQ ID NOS: 108, 109 and 144.

In one embodiment, the composition of the invention is enriched in peptides having a molecular weight of less than 10 KD. In one embodiment, the composition is depleted of cell debris.

In one embodiment, the composition of the invention is a powder.

In one embodiment, the invention comprises a composition comprising substantially all of fragments SEQ ID NO: 108-109 and 112 to 220, or anti-inflammatory variants of the fragments, or a mixture of the anti-inflammatory fragments and variants.

In one embodiment the composition is edible (comestible). In one embodiment, the composition is a food or beverage. In one embodiment, the composition is a personal care composition. In one embodiment, the composition is a pharmaceutical composition. In one embodiment, the composition is nutritional supplement. In one embodiment, the composition is solid. In one embodiment, the composition is semi-solid (i.e. a cream, gel or lotion). In one embodiment, the composition is liquid.

The invention also relates to a comestible product comprising a peptide of the invention. Preferably the comestible product is man-made.

The invention also relates to a comestible product comprising a composition of peptides of the invention. Preferably the comestible product is man-made.

Preferably, the comestible product is a food product for human or animal (mammalian) consumption.

In one embodiment the man-made comestible product is a beverage. In one embodiment the man-made comestible product is a bakery product. In one embodiment the man-made comestible product is a dairy product. In one embodiment the man-made comestible product is a snack product. In one embodiment the man-made comestible product is a baked extruded food product. In one embodiment the man-made comestible product is powdered milk. In one embodiment the man-made comestible product is an infant formula product. In one embodiment the man-made comestible product is a confectionary product. In one embodiment the man-made comestible product is a yoghurt. In one embodiment the man-made comestible product is a yoghurt drink. In one embodiment the man-made comestible product is an ice cream product. In one embodiment the man-made comestible product is a frozen food product. In one embodiment the man-made comestible product is a breakfast cereal. In one embodiment the man-made comestible product is a bread. In one embodiment the man-made comestible product is a flavoured milk drink. In one embodiment the man-made comestible product is a confectionary bar. In one embodiment the man-made comestible product is a tea or tea product. In one embodiment the man-made comestible product is a based extruded snack product. In one embodiment the man-made comestible product is a fried snack product. In one embodiment the man-made comestible product is a nutritional supplement. In one embodiment the man-made comestible product is a sports nutritional product. In one embodiment the man-made comestible product is a baby food product. In one embodiment the man-made comestible product is a speciality food product for immunocompromised individuals. In one embodiment the man-made comestible product is a food for geriatric patients.

The invention also relates to a man-made personal care composition comprising a peptide of the invention.

The invention also relates to a man-made personal care composition comprising a composition of peptides of the invention.

In one embodiment the personal care composition is a skincare product. In one embodiment the personal care composition is a haircare product. In one embodiment the personal care composition is a dentrifice product. In one embodiment the personal care composition is a perfumery product. In one embodiment the personal care composition is a deodorant product. In one embodiment the personal care composition is an anti-perspirant product. In one embodiment the personal care composition is a soap. In one embodiment the personal care composition is a liquid soap. In one embodiment the personal care composition is a cream. In one embodiment the personal care composition is a lotion. In one embodiment the personal care composition is a gel. In one embodiment the personal care composition is a powder.

The invention also relates to a peptide of the invention for use in treatment or prevention of inflammation in a mammal.

The invention also relates to a composition of peptides of the invention for use in treatment or prevention of inflammation in a mammal.

The invention also relates to a peptide of the invention for use in treatment or prevention of an inflammatory disorder in a mammal.

The invention also relates to a composition of peptides of the invention for use in treatment or prevention of an inflammatory disorder in a mammal.

In one embodiment the inflammation is symptomatic inflammation.

In one embodiment the inflammatory disorder is an inflammatory disorder of the joints. In one embodiment the inflammatory disorder is an inflammatory disorder of the cardiovascular system. In one embodiment the inflammatory disorder is an autoimmune disease. In one embodiment the inflammatory disorder is a lung and airway inflammatory disorder. In one embodiment the inflammatory disorder is an intestinal inflammatory disorder. In one embodiment the inflammatory disorder is dermatitis. In one embodiment the inflammatory disorder is acne vulgaris. In one embodiment the inflammatory disorder is psoriasis. In one embodiment the inflammatory disorder is rheumatoid arthritis. In one embodiment the inflammatory disorder is cardiovascular disease. In one embodiment the inflammatory disorder is atherosclerosis. In one embodiment the inflammatory disorder is Type I diabetes. In one embodiment the inflammatory disorder is Graves disease. In one embodiment the inflammatory disorder is Guillain-Barre disease. In one embodiment the inflammatory disorder is Lupus. In one embodiment the inflammatory disorder is Psoriatic arthritis. In one embodiment the inflammatory disorder is Ulcerative colitis. In one embodiment the inflammatory disorder is asthma. In one embodiment the inflammatory disorder is cystic fibrosis. In one embodiment the inflammatory disorder is COPD. In one embodiment the inflammatory disorder is emphysema. In one embodiment the inflammatory disorder is acute respiratory distress syndrome. In one embodiment the inflammatory disorder is colitis. In one embodiment the inflammatory disorder is inflammatory bowel disease.

The invention also relates to a peptide of the invention for use in treatment or prevention of pain in a mammal.

The invention also relates to a composition of peptides of the invention for use in treatment or prevention of pain in a mammal.

The invention also relates to a peptide of the invention for use in treatment or prevention of a metabolic disorder in a mammal.

The invention also relates to a composition of peptides of the invention for use in treatment or prevention of a metabolic disorder in a mammal.

In one embodiment, the metabolic disorder is pre-diabetes. In one embodiment, the metabolic disorder is diabetes. In one embodiment, the metabolic disorder is Type-1 diabetes. In one embodiment, the metabolic disorder is Type-2 diabetes. In one embodiment, the metabolic disorder is metabolic syndrome. In one embodiment, the metabolic disorder is obesity. In one embodiment, the metabolic disorder is diabetic dyslipidemia. In one embodiment, the metabolic disorder is hyperlipidemia. In one embodiment, the metabolic disorder is hypertension. In one embodiment, the metabolic disorder is hypertriglyceridemia. In one embodiment, the metabolic disorder is hyperfattyacidemia. In one embodiment, the metabolic disorder is hypercholerterolemia. In one embodiment, the metabolic disorder is hyperinsulinemia. In one embodiment, the metabolic disorder is MODY

The invention also relates to a peptide of the invention for use in maintaining or restoring gut health in a mammal.

The invention also relates to a composition of peptides of the invention for use in maintaining or restoring gut health in in a mammal.

The invention also relates to a peptide of the invention for use in maintaining or restoring muscle health (for example lean tissue mass) in a mammal.

The invention also relates to a composition of peptides of the invention for use in maintaining or restoring muscle health (for example lean tissue mass) in in a mammal.

The invention also relates to a pharmaceutical composition comprising a peptide of the invention in combination with a pharmaceutically acceptable carrier.

The invention also relates to a pharmaceutical composition comprising a composition of peptides of the invention in combination with a pharmaceutically acceptable carrier.

Such peptides can be used in personal care, supplement, food and pharmaceutical products to treat and maintain healthy levels of inflammation throughout the body. The present invention meets the huge need for food-derived specific peptides and peptide compositions that reduces inflammation in a way that is able to be processed by the body without completely blocking the immune response and causing autoimmune issues and other undesirable side effects. The invention is ultimately helping the 2 billion people suffering from inflammation.

The invention also relates to a comestible product, for example a food product comprising a composition of the invention, for example a dairy or non-dairy product, a solid food or a beverage, a food additive or supplement. The dairy product may be a milk, a cheese, or yoghurt. In one embodiment, the food product is a snack bar. The food product may comprise any amount of the composition of the invention, for example from 0.1% to 30% (w/w).

The peptides of the invention are used in the topical cosmetic or pharmaceutical composition of this invention at cosmetically or pharmaceutically effective concentrations to achieve the desired effect; in a preferred form with regards to the total weight of the composition, between 0.00000001% (in weight) and 20% (in weight); preferably between 0.000001% (in weight) and 15% (in weight), more preferably between 0.0001% (in weight) and 10% (in weight) and even more preferably between 0.0001% (in weight) and 5% (in weight). Ideally, the peptides of the present invention are preferably used from about 0.00001% w/w to about 0.5% w/w [0.1 to 5000 ppm], and more preferably from 0.00005 w/w to about 0.05 w/w [0.5 to 500 ppm], and most preferably from about 0.0001 w/w to about 0.01 w/w of the composition [1 to 100 ppm]. Ideally, the peptides of the present invention are preferably used from about 0.0001% w/w to about 0.004% w/w of the composition.

For compositions of peptides of the invention, a typical daily dosage may be 0.2 g to 100 g.

The dosage of compositions of the invention for use in food products and food supplements (i.e. comestible compositions) will be broadly in the 0.2-100 g/day range. In one embodiment, the daily dosage is 1-10 g/day, ideally about 3-8 g/day. In one embodiment, the daily dosage is 10-20 g/day. In one embodiment, the daily dosage is 20-30 g/day. In one embodiment, the daily dosage is 30-40 g/day. In one embodiment, the daily dosage is 10-100 g/day. In one embodiment, the daily dosage is about 5 g/day, ideally about 3-8 g/day. In one embodiment, the dosage is 2-1000 mg/day/kg body weight. In one embodiment, the dosage is 10-500 mg/day/kg body weight. In one embodiment, the dosage is 10-100 mg/day/kg body weight. In one embodiment, the dosage is 30-70 mg/day/kg body weight. The dosage of peptides of the invention for food supplements may be 0.00001 mg-0.01 mg per day or dose.

The food product may be a Food for Specific Medicinal Purposes (FSMP) which is defined as foods that are specifically formulated, processed and intended for the dietary management of diseases, disorders or medical conditions of individuals who are being treated under medical supervision. These foods are intended for the exclusive or partial feeding of people whose nutritional requirements cannot be met by normal foods. The dose may be 50-500 g per day depending on the age and condition of the patient. When administered as a food for special medicinal purpose, or medical food, the daily dosage may be 50-500 g per day.

The peptides and compositions of the invention may also be employed in the non-therapeutic treatment of inflammation. Examples of non-therapeutic treatment of inflammation include use to relieve normal, non-pathological, inflammation, for example inflammation in the muscles and joints following exercise.

The invention also provides topical composition comprising a peptide of the invention. It will be appreciated that the topical composition may comprise a plurality of peptides, fragments and/or variants. In one embodiment, the topical composition comprises substantially all the peptides. In one embodiment, the topical composition comprises substantially all the variants. The topical composition of the invention may be presented in a formulation selected from the group comprising creams, multiple emulsions, anhydrous compositions, aqueous dispersions, oils, milks, balsams, foams, lotions, gels, cream gels, hydro-alcoholic solutions, hydro-glycolic solutions, cosmetic, personal care product, hydrogels, liniments, sera, soaps, dusting powder, paste, semi solid formulations, liniments, serums, shampoo, conditioner, ointments, any rinse off formulation, talc, mousses, powders, sprays, aerosols, solutions, suspensions, emulsions, syrups, elixirs, polysaccharide films, patches, gel patches, bandages, an adhesive system, water-in-oil emulsions, oil-in-water emulsions, and silicone emulsions. In an embodiment of the current invention, the emulsion contains a lipid or oil. The emulsion may be, but is not limited to, oil-in-water, water-in-oil, water-in-oil-in-water and oil-in-water-in-silcone emulsions. The emulsion may contain a humectant. The emulsion may contain an anti-foaming agent, such as silicone. The emulsion may have any suitable viscosity. Emulsions may further contain an emulsifier and/or an anti-foaming agent. Methods of preparing an emulsion are known to a person skilled in the art.

The topical composition of the invention may be incorporated into a medical device for administration. Such a device can include but is not limited to a fabric, patch, bandage, gauge, sock, tight, underwear, dressing, glove, mask, adhesive patches, non-adhesive patches, occlusive patches and microelectric patches or suitable adhesive system. In such an embodiment, the device is in direct contact with the keratinous layer such as the skin, thus releasing the peptides of the invention. It will be understood that the topical composition may be incorporated in any suitable form as detailed herein. For example, the topical composition or peptides of the invention can be incorporated into the device or be present on the surface of the device or can be in a cream, gel or wax formulation or any suitable formulation defined herein and incorporated into the device or on the surface of the device. The device may be adapted for adhesion or attachment to the skin.

In one embodiment the device is adapted to release a constant quantity of the composition or the peptides of the invention. It will be understood that the amount of the composition contained in the sustained release system will depend, for example, on where the composition is to be administered, the kinetics and duration of the release of the composition of the invention, as well as the nature of the condition, disorder and/or disease to be treated and/or cared for. The device may be such that the composition is released by biodegradation of the device, or by friction between the device and the body, due to bodily moisture, the skin's pH or body temperature.

In an embodiment of the invention the topical composition may further comprise at least one cosmetically or pharmaceutically acceptable excipient. Excipient may be used interchangeably with functional ingredient or additive. It will be understood that although the topical compositions of the current invention can be administered alone, they will generally be administered in admixture with a cosmetic or pharmaceutical excipient. Cosmetically or pharmaceutically acceptable excipient are well known in the art and any known excipient, may be used provided that it is suitable for topical administration and is dermatologically acceptable without undue toxicity, incompatibility and/or allergic reaction.

Preferably any excipient included is present in trace amounts. The amount of excipient included will depend on numerous factors, including the type of excipient used, the nature of the excipient, the component(s) of the topical composition, the amount of active or peptide in the topical composition and/or the intended use of the topical composition. The nature and amount of any excipient should not unacceptably alter the benefits of the peptides of this invention.

In an embodiment of the invention the excipient may be a suitable diluent, carrier, binder, lubricant, suspending agent, coating agent, preservative, stabilisers, dyes, vehicle, solubilising agent, base, emollient, emulsifying agent, fragrance, humectant, and/or surfactants.

Examples of suitable diluents include, but are not limited to, any diluent disclosed in disclosed in US2014120131 or US2004132667. Examples include ethanol, glycerol and water.

Examples of suitable carriers include, but are not limited to, lactose, starch, glucose, methyl cellulose, magnesium stearate, mannitol, sorbitol and any suitable carrier disclosed in US2014120131 or US2004132667.

Examples of suitable binders include, but are not limited to, starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol and any suitable binder disclosed in US2014120131 or US2004132667.

Examples of suitable lubricants include, but are not limited to, sodium oleate, sodium stearate, magnesium stearate, sodium benzoate, sodium acetate, and sodium chloride and any suitable lubricant disclosed in US2014120131 or US2004132667.

The carrier may be any suitable carried known in the art or disclosed in US2014120131 or US2004132667. In some embodiments, the carrier may include, but is not limited to, a liquid, such as water, oils or surfactants, including those of petroleum, animal, plant or synthetic origin, polymer, oil, such as peanut oil, mineral oil, castor oil, soybean oil, alcohol, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, or digitonin. It will be understood that the carrier will be dermatologically acceptable. Preferred carriers contain an emulsion such as oil-in-water, water-in-oil, water-in-oil-in-water and oil-in-water-in-silicone emulsions. Emulsions may further contain an emulsifier and/or an anti-foaming agent.

In an embodiment of the invention, the topical composition may further comprise one or more additional ingredients. The topical composition of the invention may be administered consecutively, simultaneously or sequentially with the one or more other additional agents. Such additional ingredients may be those of benefit to include in a topical composition, or of benefit depending on the intended use of the topical composition. The additional ingredient may be active or functional or both.

Examples of such additional ingredients include, but are not limited to, one or more of cleaning agents, conditioning agents, sunscreen, pigment, moisturiser, thickening agents, gelling agents, essential oil, astringents, pigments, anti-caking agent, anti-foaming agent, binders, additives, buffers, chelating agents, external analgesics, film formers or materials, bulking agents, polymers, opacifying agents, pH adjusters, propellants, reducing agents, sequestrants, skin bleaching and lightening agents, skin conditioning agents, aloe vera, healing agents, soothing agents, smoothing agents, pantothenic acid, treating agents, thickeners, vitamins. colourants, pharmaceuticals, antiseptic agents, antifoaming agents, buffering agents, astringents, polymers, pH adjuster, deodorant or any other dermatologically acceptable carrier or surfactant.

It is to be understood that additional ingredients listed may provide more than one benefit. The classification given herein is for clarity and convenience only and not intended to limit the additional ingredient to that particular application or category listed.

Any additional ingredients should be suitable for application to the skin without undue toxicity, incompatibility and/or allergic reaction.

In some embodiments, the additional ingredient has glucose transport activity or aids glucose transport activity. In some embodiments, the additional ingredient has anti-inflammatory activity or aids anti-inflammatory activity. In some embodiments, the additional ingredient has anti-aging activity or aids anti-aging activity. In some embodiments, the additional ingredient is for keratinous layer health and/or development, skin health and/or development, and/or muscle health, recovery and/or development. The active agent may be a pharmacological enhancer. Such active agents are known and available on the market. In such cases, the topical composition of the invention may be administered consecutively, simultaneously or sequentially with the one or more other active agents.

In some embodiments, the additional ingredient may be farnesol ([2E, 6E],-3,7,11,-trimethyl-2,6,10, dodecatrien-1-ol), phytantriol (3, 7, 11, 15, tetramethylhexadecane-1, 2, 3,-triol), desquamation actives, enzymes, enzyme inhibitors, enzyme activators, botanical extracts and marine extracts, anti-acne actives, anti-wrinkle or anti atrophy actives, anti-oxidant/radical scavengers, chelators, flavonoids, anti-inflammatory agents, anti-cellulite agents, topical anaesthetics, tanning actives, skin lightening agents, skin healing agents, bisabolol, antimicrobial or antifungal active, sunscreen actives, particulate material, conditioning agents, structuring agents, thickening agent,

The desquamation active may be any suitable agent that enhances the skin appearance or texture of the skin and is as disclosed in US2014120131 or US2004132667.

Examples of anti-acne actives are as disclosed in US2014120131 or US2004132667 and include, resorcinol, salicylic acid, erythromycin, zine, sulfur, benzoyl peroxides.

Examples of thickening agents are as disclosed in US2014120131 or US2004132667 and include carboxylic acid polymers, crosslinked polyacrylate polymers, polyacrylamide polymers, polysaccharides.

Examples of conditioning agents are as disclosed in US2014120131 or US2004132667 and include humectants, moisturiser or skin conditioner.

Examples of structuring agents are as disclosed in US2014120131 or US2004132667 and include any agent that provide rheological characteristics to the composition and contributes to the stability of the composition.

Any suitable antimicrobial or antifungal active may be used and examples are as disclosed in US2014120131 or US2004132667. Such actives are capable of destroying microbes, preventing growth or action of microbes. Examples include but are not limited to β-lactam drugs, quinolone drugs, tetracycline, erythromycin, streptomycin sulfate, salicylic acid, benzoyl peroxide.

Examples of a particulate material include metallic oxide. Examples of anti-cellulite agents include xanthine agents. Examples of tanning actives includes 1,3-dihydroxy-2-propanone and those disclosed in US2014120131 or US2004132667. Examples of topical anaesthetics include benzocaine, lidocaine and bupivacaine and those disclosed in US2014120131 or US2004132667.

Examples of skin lightening agents include any agent known in the art such as kojic acid, ascorbic acid and those disclosed in US2014120131 or US2004132667.

Examples of sunscreen actives include any suitable organic or inorganic sunscreen active.

Examples include metallic oxides, 2-ethylhexyl-p-methoxycinnamate and those disclosed in US2014120131 or US2004132667.

Examples of skin healing agents includes panthenoic acid as disclosed in US2014120131 or US2004132667.

Examples of anti-inflammatory agents include any agent that enhances the skin appearance, tone or colour and include but are not limited to corticosteroids, hydrocortisone, non-steroidal agents such as ibuprofen and aspirin and those disclosed in US2014120131 or US2004132667.

Examples of flavonoids includes flavanones, methoxy flavonones, unsubstituted chalcone and mixtures thereof and those disclosed in US2014120131 or US2004132667.

Examples of enzymes include lipases, proteases, catalase, super oxide-dismutase, amylase, peroxidase, glucuronidase, ceramidases, hyaluronidases. Examples of enzyme inhibitors include trypsine inhibitors, Bowmann Birk inhibitors, chymotrypsin inhibitors, botanical extracts, flavonoids, quercetin chalcone and those disclosed in US2014120131 or US2004132667 and mixtures thereof. Examples of enzyme activators include coenzyme A, Q10 (ubiquinone), glycyrrhizin, berberine, chrysin and those disclosed in US2014120131 or US2004132667 and mixtures thereof.

Examples of anti-wrinkle or anti atrophy actives include sulfur containing D and L amino acids, particular, N-acyl derivatives such as N-acetyl-L-cysteine, hydroxyl acids, phytic acid, lipoic acid, lysophosphatidic acid, skin peel agents, vitamin B₃, retinoids and those disclosed in US2014120131 or US2004132667 and mixtures thereof.

The anti-oxidant/radical scavenger agent may be any agent that is useful for providing protection against UV radiation or other environmental agents which may cause skin damage such as those disclosed in US2014120131 or US2004132667. Examples of anti-oxidant/radical scavengers include ascorbic acid, its salts and derivatives (vitamin C), tocopherol its salts and derivatives (vitamin E), butylated hydroxyl benzoic acids and their salts, peroxides, gallic acids and alkyl esters, sorbic acid, lipoic acid, amines, lycine pidolate, arginine pilolate, nordihydroguaiaretic acid, bioflavonoids, curcumin, lysine, methionine, proline, superoxide dismutase, silymarin, tea extracts and mixtures thereof.

Examples of chelators include EDTA, NTA, hydoxamic acids, phytic acid, lactoferrin and those disclosed in US2014120131 or US2004132667 and mixtures thereof. A chelator means an agent capable of removing a metal ion by forming a complex so that the metal ion cannot participate in or catalyse chemical reactions. A chelator is useful for protection against UV radiation or other environmental agents that can cause skin damage.

It will be appreciated that a plurality of additional ingredients may be added. The amount of the additional ingredient may be from about 0.001% to about 50% weight of the composition, preferably, about 0.01% to about 20%, preferably about 0.1% to about 10%, about 0.5% to about 10%, about 1% to about 5%, preferably 2% weight of the composition. The amount of additional ingredient included will depend on numerous factors, including the type of additional ingredient used, the nature of the additional ingredient, the component(s) of the topical composition, the amount of active or peptide in the topical composition and/or the intended use of the topical composition. The nature and amount of any additional ingredient should not unacceptably alter the benefits of the peptides of this invention.

The topical composition may be alcohol free.

In some embodiments of the invention, the composition further comprises one or more additional active agents, in addition to the peptide of the invention (also known as the active of the composition). In addition, or alternatively, the composition may be administered with one or more other additional active agents. Typical said additional active agent is present in trace amounts only. In some embodiments, there may be no additional active agent present in the composition. The amount of additional active agent included will depend on numerous factors, including the type of additional active agent used, the nature of the additional active agent, the component(s) of the topical composition, the amount of active or peptide in the topical composition and/or the intended use of the topical composition. The nature and amount of any additional active agent should not unacceptably alter the benefits of the peptides of this invention.

It is to be understood that an ingredient that is considered to be an “active” ingredient in one product may be a “functional” or “excipient” ingredient in another and vice versa. It will also be appreciated that some ingredients play a dual role as both an active ingredient and as a functional or excipient ingredient.

Examples of the additional active agents include glucose transport promoting drugs, skin supplement, agent for treatment and/or care of the skin, anti-inflammatory agent, an anti-aging agent, a cellular growth promoting agent and pharmacological enhancers. Such agents are well known in the art and it will be appreciated that any suitable additional active agent may be used. Additional active agents for treatment and/or care of the skin may include collagen synthesis agents, retinoids, exfoliating agents, anti-cellulite agents, elastase inhibiting agents, melanin synthesis stimulating or inhibiting agents, self-tanning agents, antiaging agents, antimicrobial agents, antifungal agents, fungistatic agents, bactericidal agents, and healing agents. Active agents also include anti-inflammatory agents.

Any additional active agent should be suitable for application to the skin without undue toxicity, incompatibility and/or allergic reaction.

It will be understood that the classification given herein is for clarity and convenience only and not intended to limit the additional ingredient, excipient, or active to that particular application or category listed.

In a particularly preferred embodiment, the methods and uses of the invention involve administration of a peptide or composition of the invention in combination with one or more other active agents, for example, existing growth promoting drugs or pharmacological enhancers available on the market. In such cases, the compounds of the invention may be administered consecutively, simultaneously or sequentially with the one or more other active agents.

The effect of the current invention is accomplished by topical application or administration of the topical composition of the invention described herein to a person, animal or a patient in need of treatment or care. Topical delivery preferably means delivery to a keratinous layer such as the skin, hair and/or nails, but can also mean delivery to a body lumen lined with epithelial cells, for example the lungs or airways, the gastrointestinal tract, the buccal cavity. The effect may be confined to the surface of the skin or may be within the skin or a combination of both.

The topical composition of the invention is administered in a cosmetically or pharmaceutically effective amount. In other words, in an amount that is non-toxic but sufficient amount to provide the desired effect. It will be appreciated that a person skilled in the art would be capable of determining an appropriate dose of the topical compositions of the invention to administer without undue experimentation. Alternatively, a physician will determine the actual dose that is most suitable for a patient depending on the particular condition, disease or disorder to be treated or cared for and the age, body weight and/or health of the person. It will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can, of course, be individual instances where higher or lower dosage ranges are merited, and such are within the scope of this invention. For example, the composition may be administered at a dose of from 0.01 to 50 mg/kg body weight, such as from 0.1 to 30 mg/kg, more preferably from 0.1 to 20 mg/kg body weight, more preferably from 0.1 to 10 mg/kg body weight, preferably 0.1 to 5mg/kg body weight. In an exemplary embodiment, one or more doses of 10 to 300 mg/day or more preferably, 10 to 150 mg/day, will be administered to the patient. The amount and the frequency is as best suited to the purpose. The frequency of application or administration can vary greatly, depending on the needs of each subject, with a recommendation of an application or administration range from once a month to ten times a day, preferably from once a week to four times a day, more preferably from three times a week to three times a day, even more preferably once or twice a day.

In preferred embodiments, repeated use of the topical composition is provided. The topical composition may be applied by, but not limited to, rubbing, or massaging into the keratinous tissue, skin or area of the body to be treated or cared for. In some embodiments, the composition is left on or not removed from the area of the body. In other embodiments, the composition is removed after a period of time, such as, but not limited to, from about 2 minutes to 60 minutes, from about 5 minutes to about 30 minutes, preferably from about 10 minutes to about 20 minutes. The composition may be removed immediately after application. In some embodiments of the current invention, the composition of the invention may be applied to an area to be treated by means to achieve a greater penetration of the composition and/or peptide of the invention, such as, but not limited to, iontophoresis, sonophoresis, electroporation, microelectric patches, mechanical pressure, osmotic pressure gradient, occlusive cure, microinjections or needle-free injections by means of pressure, such as injections by oxygen pressure, or any combination thereof.

The peptides of the invention are used in the topical cosmetic or pharmaceutical composition of this invention at cosmetically or pharmaceutically effective concentrations to achieve the desired effect; in a preferred form with regards to the total weight of the composition, between 0.00000001% (in weight) and 20% (in weight); preferably between 0.000001% (in weight) and 15% (in weight), more preferably between 0.0001% (in weight) and 10% (in weight) and even more preferably between 0.0001% (in weight) and 5% (in weight).

In some embodiments of the current invention, the composition may be delivered via any one of liposomes, mixed liposomes, oleosomes, niosomes, ethosomes, millicapsules, capsules, macrocapsules, nanocapsules, nanostructured lipid carriers, sponges, cyclodextrins, vesicles, micelles, mixed micelles of surfactants, surfactant-phospholipid mixed micelles, millispheres, spheres, lipospheres, particles, nanospheres, nanoparticles,milliparticles, solid nanopartciles as well as microemulsions including water-in-oil microemulsions with an internal structure of reverse micelle and nanoemulsions microspheres, microparticles.

A variety of methods are available for preparing liposomes. See, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91/17424, Deamer & Bangham, Biochim. Biophys. Acta 443:629-634 (1976); Fraley, et al., PNAS 76:3348-3352 (1979); Hope et al., Biochim. Biophys. Acta 812:55-65 (1985); Mayer et al., Biochim. Biophys. Acta 858:161-168 (1986); Williams et al., PNAS 85:242-246 (1988); Liposomes (Ostro (ed.), 1983, Chapter 1); Hope et al., Chem. Phys. Lip. 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vehicles and ether fusion methods, all of which are well known in the art.

These delivery systems may be adapted to achieve a greater penetration of the compound and/or peptides of the invention. This may improve pharmacokinetic and pharmacodynamics properties. The delivery system may be a sustained release system wherein the compound or peptide of the invention is gradually released during a period of time and preferably with a constant release rate over a period of time. The delivery systems are prepared by methods known in the art. The amount of peptide contained in the sustained release system will depend on where the composition is to be delivered and the duration of the release as well as the type of the condition, disease and/or disorder to be treated or cared for.

The topical composition of the invention may be for human or animal usage in human and veterinary medicine.

The topical composition of the invention may be used for pharmaceutical, personal care and/or cosmetic uses.

The composition can be used to treat or care for any disease, disorder or condition of the skin, including but not limited to, psoriasis, dermatitis, allergic dermatitis, eczema, spongiosis, edema, skin cancer, ulcers, acne, scars, cellulitis, elastosis, keratosis, rosacea, varicose veins, inflammatory disorders.

The topical composition may be used to for treating or caring for visible signs of aging including but not limited to wrinkles, stretch marks and dark circles, dryness, fine lines, age spots, red blotches, sagging skin, and conditions caused by sun exposure including sunburn, stress, pollution and/diet. The topical composition may also be used for delaying, slowing or inhibiting the skins or the onset of aging. The composition may be administered by a medical device, such as a plaster or a patch as described herein.

The topical composition may be used to treat or care for a wound in a mammal. In another embodiment, the topical composition is for use in the treatment or prevention of a disease or condition characterised by damaged epithelial cells or tissue, and/or damaged dermal or epithelial cells or tissue. The disease may be but is not limited to cancer and trauma.

The topical composition may be used to treat or care for any muscle condition, to improve, muscle status in a mammal, to promote recovery of muscle, typically following exercise, to maintain or restore muscle health (for example lean tissue mass) in a mammal, to enhance physical performance, in treatment or prevention of a disease or condition characterised by lethargy or low energy levels.

The topical composition may be used to promote growth of a tissue, promote growth of epithelial tissue, promote growth of skin, promote growth of an organ, promote growth of an organism. The skin can have a normal pathology and/or an abnormal pathology.

The topical composition may also be used to treat or care for any inflammatory disorder.

A further aspect of the invention relates to a pharmaceutical composition comprising a peptide of the invention or a composition of peptides of the invention, admixed with one or more pharmaceutically acceptable diluents, excipients or carriers. Even though the peptides and compositions of the present invention can be administered alone, they will generally be administered in admixture with a pharmaceutical carrier, excipient or diluent, particularly for human therapy. The pharmaceutical compositions may be for human or animal usage in human and veterinary medicine. Examples of such suitable excipients for the various different forms of pharmaceutical compositions described herein may be found in the “Handbook of Pharmaceutical Excipients, 2^{nd} Edition, (1994), Edited by A Wade and P J Weller. In particular, formulations for topical delivery are described in Topical drug delivery formulations edited by David Osborne and Antonio Aman, Taylor & Francis, the complete contents of which are incorporated herein by reference. Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The peptide or composition of the invention may be adapted for topical, oral, rectal, parenteral, intramuscular, intraperitoneal, intra-arterial, intrabronchial, subcutaneous, intradermal, intravenous, nasal, vaginal, buccal or sublingual routes of administration. For oral administration, particular use is made of compressed tablets, pills, tablets, gellules, drops, and capsules. Preferably, these compositions contain from 1 to 250 mg and more preferably from 10-100 mg, of active ingredient per dose. Other forms of administration comprise solutions or emulsions which may be injected intravenously, intra-arterial, subcutaneously, intradermally, intraperitoneally or intramuscularly, and which are prepared from sterile or sterilisable solutions. The pharmaceutical compositions of the present invention may also be in form of suppositories, vaginal rings, pessaries, suspensions, emulsions, lotions, ointments, creams, gels, sprays, solutions or dusting powders. The composition of the invention may be formulated for topical delivery. Topical delivery generally means delivery to the skin, but can also mean delivery to a body lumen lined with epithelial cells, for example the lungs or airways, the gastrointestinal tract, the buccal cavity. In particular, formulations for topical delivery are described in Topical drug delivery formulations edited by David Osborne and Antonio Aman, Taylor & Francis, the complete contents of which are incorporated herein by reference. Compositions or formulations for delivery to the airways are described in O'Riordan et al (Respir Care, 2002, November 47), EP2050437, WO2005023290, US2010098660, and US20070053845. Composition and formulations for delivering active agents to the iluem, especially the proximal iluem, include microparticles and microencapsulates where the active agent is encapsulated within a protecting matrix formed of polymer or dairy protein that is acid resistant but prone to dissolution in the more alkaline environment of the ileum. Examples of such delivery systems are described in EP1072600.2 and EP13171757.1. An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention. Depending upon the need, the agent may be administered at a dose of from 0.01 to 30 mg/kg body weight, such as from 0.1 to 10 mg/kg, more preferably from 0.1 to 1 mg/kg body weight. In an exemplary embodiment, one or more doses of 10 to 300 mg/day or more preferably, 10 to 150 mg/day, will be administered to the patient for the treatment of an inflammatory disorder.

In a particularly preferred embodiment, the methods and uses of the invention involve administration of a peptide or composition of the invention in combination with one or more other active agents, for example, existing anti-inflammatory drugs or pharmacological enhancers available on the market. In such cases, the compounds of the invention may be administered consecutively, simultaneously or sequentially with the one or more other active agents.

In one embodiment of the invention, the peptide of the invention may be administered in the form of a conjugate comprising the peptide, and may optionally include a linker, and a partner molecule, for example a protein such as an antibody molecule intended to increase the half-life of the conjugate in-vivo. In one embodiment, the peptide may be modified to substitute one or more amino acids with amino acids employed to attach partner molecules. For example, an amino acid may be substituted with a lysine residue for the purpose of conjugating a partner molecule such as a PEG molecule.

### Definitions

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

The term “mammal” should be understood to mean a higher mammal, especially a human. However, the term also includes non-mammalian animals such as fish.

The term “peptide” used herein refers to a polymer composed of up to 50 amino acid monomers typically via peptide bond linkage. The peptide may be 3-50 amino acids in length. The peptide may be 4 to 50 amino acids in length. The peptide may be 5-50 amino acids in length. The peptide may be 7 to 50 amino acids in length. Peptides (including fragments and variants thereof) of and for use in the invention may be generated wholly or partly by chemical synthesis or by expression from nucleic acid. For example, the peptides of and for use in the present invention can be readily prepared according to well-established, standard liquid or, preferably, solid-phase peptide synthesis methods known in the art (see, for example, J. M. Stewart and J. D. Young, Solid Phase Peptide Synthesis, 2nd edition, Pierce Chemical Company, Rockford, Illinois (1984), in M. Bodanzsky and A. Bodanzsky, The Practice of Peptide Synthesis, Springer Verlag, New York (1984). When necessary, any of the peptides employed in the invention can be chemically modified to increase their stability. A chemically modified peptide or a peptide analog includes any functional chemical equivalent of the peptide characterized by its increased stability and/or efficacy in vivo or in vitro in respect of the practice of the invention. The term peptide analog also refers to any amino acid derivative of a peptide as described herein. A peptide analog can be produced by procedures that include, but are not limited to, modifications to side chains, incorporation of unnatural amino acids and/or their derivatives during peptide synthesis and the use of cross-linkers and other methods that impose conformational constraint on the peptides or their analogs. Examples of side chain modifications include modification of amino groups, such as by reductive alkylation by reaction with an aldehyde followed by reduction with NaBH₄; amidation with methylacetimidate; acetylation with acetic anhydride; carbamylation of amino groups with cyanate; trinitrobenzylation of amino groups with 2, 4, 6, trinitrobenzene sulfonic acid (TNBS); alkylation of amino groups with succinic anhydride and tetrahydrophthalic anhydride; and pyridoxylation of lysine with pyridoxa-5′-phosphate followed by reduction with NABH₄. The guanidino group of arginine residues may be modified by the formation of heterocyclic condensation products with reagents such as 2,3-butanedione, phenylglyoxal and glyoxal. The carboxyl group may be modified by carbodiimide activation via o-acylisourea formation followed by subsequent derivatization, for example, to a corresponding amide. Sulfhydryl groups may be modified by methods, such as carboxymethylation with iodoacetic acid or iodoacetamide; performic acid oxidation to cysteic acid; formation of mixed disulphides with other thiol compounds; reaction with maleimide; maleic anhydride or other substituted maleimide; formation of mercurial derivatives using 4-chloromercuribenzoate, 4-chloromercuriphenylsulfonic acid, phenylmercury chloride, 2-chloromercuric-4-nitrophenol and other mercurials; carbamylation with cyanate at alkaline pH. Tryptophan residues may be modified by, for example, oxidation with N-bromosuccinimide or alkylation of the indole ring with 2-hydroxy-5-nitrobenzyl bromide or sulphonyl halides. Tryosine residues may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate. Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. Peptide structure modification includes the generation of retro-inverso peptides comprising the reversed sequence encoded by D-amino acids.

“Modified peptide”: In an embodiment of the invention the peptide is a modified peptide. The term modified peptide is used interchangeably with the term derivative of the peptide. The modified peptide includes a peptide which has been substituted with one or more groups as defined herein. The modification may be any modified that provides the peptides and or the composition of the invention with an increased ability to penetrate a cell. The modification may be any modification that increases the half-life of the composition or peptides of the invention. In one embodiment, the group is a protecting group. The protecting group may be an N-terminal protecting group, a C-terminal protecting group or a side-chain protecting group. The peptide may have one or more of these protecting groups.The person skilled in the art is aware of suitable techniques to react amino acids with these protecting groups. These groups can be added by preparation methods known in the art, for example the methods as outlined in paragraphs to of US2014120141. The groups may remain on the peptide or may be removed. The protecting group may be added during synthesis. In an embodiment of the invention the peptides may be substituted with a group selected from one or more straight chain or branched chain, long or short chain, saturated, or unsaturated, substituted with a hydroxyl, amino, amino acyl, sulfate or sulphide group or unsubstituted having from 1 to 29 carbon atoms. N-acyl derivatives include acyl groups derived from acetic acid, capric acid, lauric acid, myristic acid, octanoic acid, palmitic acid, stearic acid, behenic acid, linoleic acid, linolenic acid, lipoic acid, oleic acid, isosteric acid, elaidoic acid, 2-ethylhexaneic acid, coconut oil fatty acid, tallow fatty acid, hardened tallow fatty acid, palm kernel fatty acid, lanolin fatty acid or similar acids. These may be substituted or unsubstituted. When substituted they are preferably substituted with hydroxyl, or sulphur containing groups such as but not limited to SO₃H, SH, or S—S. In an embodiment of the current invention, the peptide is R₁—X—R₂. R₁ and/or R₂ groups respectively bound to the amino-terminal (N-terminal) and carboxyl-terminal (C-terminal) of the peptide sequence. In one embodiment, the peptide is R₁—X. Alternatively, the peptide is X—R₂. Preferably, R₁ is H, C₁₋₄ alkyl, acetyl, benzoyl or trifluoroacetyl;. X is the peptide of the invention; R₂ is OH or NH₂. In an embodiment, R₁ is selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, substituted or unsubstituted aralkyl, Tert-butyloxycarbonyl, 9-fluorenylmethyloxycarbonyl (Fmoc) and R₅—CO—, wherein R₅ is selected from the group formed by H, a non-cyclic substituted or unsubstituted aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl; R₂ is selected from the group formed by —NR₃R₄, —OR₃ and —SR₃, wherein R₃ and R₄ are independently selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl; and with the condition that R₁ and R₂ are not α-amino acids. In accordance with another preferred embodiment, R₂ is —NR₃R₄, —OR₃ or —SR₃ wherein R₃ and R₄ are independently selected from the group formed by H, substituted or unsubstituted C₁-C₂₄ alkyl, substituted or unsubstituted C₂-C₂₄ alkenyl, Tert-10 butyloxycarbonyl, 9-fluorenylmethyloxycarbonyl (Fmoc), substituted or unsubstituted C₂-C₂₄ alkynyl, substituted or unsubstituted C₃-C₂₄ cycloalkyl, substituted or unsubstituted C₅-C₂₄ cycloalkenyl, substituted or unsubstituted C₈-C₂₄ cycloalkynyl, substituted or unsubstituted C₆-C₃₀ aryl, substituted or unsubstituted C₇-C₂₄ aralkyl, substituted or unsubstituted heterocyclyl ring of 3-10 members, and substituted or unsubstituted heteroarylalkyl of 2 to 24 carbon atoms and 1 to 3 atoms other than carbon wherein the alkyl chain is of 1 to 6 carbon atoms. Optionally, R₃ and R₄ can be bound by a saturated or unsaturated carbon-carbon bond, forming a cycle with the nitrogen atom. More preferably R₂ is —NR₃R₄ or —OR₃, wherein R₃ and R₄ are independently selected from the group formed by H, substituted or unsubstituted C₁-C₂₄ alkyl, substituted or unsubstituted C₂-C₂₄ alkenyl, substituted or unsubstituted C₂-C₂₄ alkynyl, substituted or unsubstituted C₃-C₁₀ cycloalkyl, substituted or unsubstituted C₆-C₁₅ aryl and substituted or unsubstituted heterocyclyl of 3-10 members, substituted or unsubstituted heteroarylalkyl with a ring of 3 to 10 members and an alkyl chain of 1 to 6 carbon atoms. More preferably R₃ and R₄ are selected from the group formed by H, methyl, ethyl, hexyl, dodecyl, or hexadecyl. Even more preferably R₃ is H and R₄ is selected from the group formed by H, methyl, ethyl, hexyl, dodecyl, or hexadecyl. In accordance with an even more preferred embodiment, R₂ is selected from —OH and —NH₂. In accordance with another embodiment of this invention R₁ is selected from the group formed by H, acetyl, lauroyl, myristoyl or palmitoyl, and R₂ is —NR₃R₄ or —OR₃ wherein R₃ and R₄ are independently selected from H, methyl, ethyl, hexyl, dodecyl and hexadecyl, preferably R₂ is —OH or —NH₂. More preferably, R₁ is acetyl or palmitoyl and R₂ is —NH₂. In a preferred embodiment, the acyl group is bound to the N-terminal end of at least one amino acid of the peptide. In an embodiment of the invention, the peptide is modified to comprise a side chain protecting group. The side chain protecting group may be one or more of the group comprising benzyl or benzyl based groups, t-butyl-based groups, benzyloxy-carbonyl (Z) group, and allyloxycarbonyl (alloc) protecting group. The side chain protecting group may be derived from an achiral amino acid such as achiral glycine. The use of an achiral amino acid helps to stabilise the resultant peptide and also facilitate the facile synthesis route of the present invention. Preferably, the peptide further comprises a modified C-terminus, preferably an amidated C-terminus. The achiral residue may be alpha-aminoisobutyric acid (methylalaine). It will be appreciated that the specific side chain protecting groups used will depend on the sequence of the peptide and the type of N-terminal protecting group used.

“Conjugate”: In one embodiment of the invention the peptide is conjugated, linked or fused to a binding partner, for example one or more polyethylene glycol polymers or other compounds, such as molecular weight increasing compounds or lipophilic groups. The molecular weight increasing compound is any compound that will increase the molecular weight, typically by 10% to 90%, or 20% to 50% of the resulting conjugate and may have a molecular weight of between 200 and 20,000, preferably between 500 and 10,000. The molecular weight increasing compound may be PEG, any water-soluble (amphiphilic or hydrophilic) polymer moiety, homo or co-polymers of PEG, a monomethyl-subsitututed polymer of PEG (mPEG) and polyoxyethylene glycerol (POG), polyamino acids such as poly-lysine, poly-glutamic acid, poly-aspartic acid, particular those of L conformation, pharmacologically inactive proteins such as albumin, gelatin, a fatty acid, olysaccharide, a lipid amino acid and dextran. The polymer moiety may be straight chained or branched and it may have a molecular weight of 500 to 40000 Da, 5000 to 10000 Da, 10000 to 5000, Da. The compound (binding partner) may be any suitable cell penetrating compound, such as tat peptide, penetratin, pep-1. The compound (binding partner) may be an antibody molecule. The compound (binding partner) may be a lipophilic moiety or a polymeric moiety. The lipophilic substituent and polymeric substituents are known in the art. The lipophilic substituent includes an acyl group, a sulphonyl group, an N atom, an O atom or an S atom which forms part of the ester, sulphonyl ester, thioester, amide or sulphonamide. The lipophilic moiety may include a hydrocarbon chain having 4 to 30 C atoms, preferably between 8 and 12 C atoms. It may be linear or branched, saturated or unsaturated. The hydrocarbon chain may be further substituted. It may be cycloalkane or heterocycloalkane. The peptide may be modified at the N-terminal, C-terminal or both. The polymer or compound (binding partner) is preferably linked to an amino, carboxyl or thio group and may be linked by N-termini or C-termini of side chains of any amino acid residue. The polymer or compound (binding partner) may be conjugated to the side chain of any suitable residue. The polymer or compound (binding partner) may be conjugated via a spacer. The spacer may be a natural or unnatural amino acid, succinic acid, lysyl, glutamyl, asparagyl, glycyl, beta-alanyl, gamma-amino butanoyl. The polymer or compound (binding partner) may be conjugated via an ester, a sulphonyl ester, a thioester, an amide, a carbamate, a urea, a sulphonamide. A person skilled in the art is aware of suitable means to prepare the described conjugate.

“Fragment” means a segment of a protein, typically selected from SEQ ID Nos: 1 to 16 and 353-355, and particularly from a region of those proteins selected SEQ ID NOS: 17 to 70. In one embodiment, the fragment is typically 3 to 37 contiguous amino acids in length. In one embodiment, the fragment is typically 5 to 37 contiguous amino acids in length. In one embodiment, the fragment is typically 5 to 37 contiguous amino acids in length. The fragment generally having a charge of between −9 and +3; typically a c-terminal amino acid that typcially is not cysteine (C) or methionine (M); and typically an n-terminal amino acid that typcially is not cysteine (C), histidine (H), proline (P) or threonine (T). The charge of a peptide, fragment or region is determined using the method of Cameselle, J. C., Ribeiro, J. M., and Sillero, A. (1986). Derivation and use of a formula to calculate the net charge of acid-base compounds. Its application to amino acids, proteins and nucleotides. Biochem. Educ. 14, 131-136.

The term “natural” as applied to a peptide means a peptide that includes (a) a fragment of a plant protein, typically rice or pea protein, or variants of pea protein including lentil, sweet pea, or chick pea or variants of rice protein including oat, grass, corn, wild rice and bananas, or (b) a variant of the fragment of a plant protein, for example a fragment of a homolog of the plant protein. The peptides or fragments of the invention may be isolated from plant proteins or made synthetically.

“C-terminal domain” as applied to a fragment means the first three amino acids at the c-terminus of the fragment.

“N-terminal domain” as applied to a fragment means the last three amino acids at the n-terminus of the fragment.

“Bioactive” as applied to a peptide or fragment means having a health promoting effect when administered a mammal, for example one or more of glucose transport promoting, anti-bacterial, anti-inflammatory, or cellular growth or proliferation promoting. In one embodiment, the term “bioactive” means anit-inflammatory.

“Glucose transport promoting” or “glucose transport promoting activity” as applied to a peptide or variant or fragment means a peptide, variant or fragment that is capable of increasing GLUT4 translocation into skeletal muscle compared with an untreated control when employed at a concentration of 2 μM in the following in-vitro assay. L6-GLUT4myc cells were grown in 10% FBS and 2 μg/ml blasticidin. Cells were grown for 48-72 hours before being seeded in 24-well plates at 15,000 cells per well in 2% FBS and allowed to differentiate for 6 to 8 days prior to experimentation. L6-GLUT4myc cells were serum-starved for three hours prior to incubation with 100 nM of insulin for 30 mins, or 200, 20, 2.0 and 0.2 μM of SP, and 2, 1, 0.5 and 0.25 mg/ml of peptide composition for 3 hours respectively. A 3 hour incubation period was selected based on previous findings identifying that incubation with branch chain amino acid containing di-peptides for 3 hours increases glucose uptake in L6 myotubes 1. Treatments were staggered in order to determine GLUT4myc translocation at the same time point. The quantity of myc-tagged GLUT4 at the cell surface was measured by antibody-coupled colorimetric assay. Briefly, after incubation with either insulin for 30 mins or synthetic peptide or peptide composition for 3 hours respectively, L6-GLUT4myc cells were fixed via incubation with 3% paraformaldehyde (PFA). A 0.1 M glycine solution was then added to quench PFA and cells were blocked with 5% goat serum. The myotube monolayer was exposed to anti-myc antibody and then incubated with peroxidase conjugated donkey anti-mouse IgG. 1 mL of o-phenylenediamine dihydrochloride (OPD) reagent was added to each well and this reaction was stopped by adding 250 μl/well of 3 M HCL. To determine GLUT4 translocation to cell surface, a measured aliquot of each condition was determined spectrophotometrically on a plate reader using absorbance at 492 nm. Preferably the peptide or fragment is capable of increasing GLUT4 translocation compared with an untreated control by at least 50% (i.e a relative unit increase in GLUT4 translocation of 1% to 1.5%).

“Antibacterial” or “antibacterial activity” as applied to a peptide or fragment means a peptide or fragment that is capable of visibly inhibiting the growth of a bacteria in the following agar-plate based growth inhibition assay: Peptide stock=5 mg/mL dissolved in DMSO. Bacterial inoculums were adjusted to McFarland 0.5 standard and MHA plates swabbed. Blank disks were placed in the plates and 10 μL of each compound (at 64 μg/mL—maximum concentration tested) added. Plates were incubated at 37° C. for 16-18 hours. Appropriate controls (DMSO; Mueller-Hinton media alone; and two antibiotic discs—ciprofloxacin and tetracycline) were also performed.

“Anti-inflammatory” as applied to a peptide or fragment means a peptide or fragment that is capable of significantly reducing the secretion of TNFα by LPS-stimulated J774.2 macrophages (compared with untreated LPS-stimulated J774.2 macrophages) when the macrophages are treated with 100 μM of the peptide or fragment. J774.2 macrophages were treated with 100 μM of synthetic peptide for 24 hours and then stimulated with (A) LPS (10 ng/ml) for five hours or (B) LPS (10 ng/ml) for 5 hours followed by ATP (5 mM) for one hour. Supernatant was collected and levels of TNFα were determined by ELISA.

“Cellular growth promoting” or “cellular growth or proliferation promoting” as applied to a peptide or fragment means a peptide or fragment that is capable of increasing elastin production or cellular proliferation of human skin treated with a 20 μM solution of peptide or fragment in the following assay. Skin explants were prepared from abdominal plastic surgery. Some explants were delipidated with alcohol to obtain a dehydrated skin. These explants were maintained in maintenance medium supplied by the provider Biopredic International for 5 days. Test items are applied twice per day with 5 μL per explant. At the end of the test, viabilities controls are realized with the MTT on two explants, the third explant is fixed in the formaldehyde 4% for histology and cell staining. For each time of analysis (D1 and D5), histologies on delipidated explants, treated explants with test items, the DMSO 0.3% control and water control, are performed. After receipt in the laboratory, each skin explant in the maintenance medium is delipidated with 5 μL alcohol during 3 hours. After 3 hours, all skin explants are treated two per day with test items, and they are incubated at 37° C.+/−2° C., 5% CO2 for 1 day or 5 days. Integrity of the system is realized at day 1 and day 5 with a viability control with MTT. Histology is realized by the laboratory Gredeco and the immunostaining to elastin and Ki67 are realized by the same laboratory. Immunostaining to filaggrin is realized by the laboratory Intertek. The detection of elastin (rabbit monoclonal antibody, clone P15502, LSBio) is performed using an immunoperoxidase technique two layers (ABC kit, Vector Laboratories) and revealed by AEC (3-amino-9-éthylcarbazole). The immunohistochemical staining intensity in the elastic fibers is evaluated using a semi-quantitative histological score. Epithelial proliferation was analyzed by immunohistochemistry using anti-Ki67 antibody. Immunodetection was performed using an indirect immunoperoxidase technique three layers, amplified (DAKO kit) and revealed by AEC (3-Amino-9-ethylcarbazole). Counting the number of labeled cells (keratinocytes of the basal layer of the epidermis) is performed and provides the total number of basal cells to calculate the % of labeled cells. The specific staining of filaggrin is performed with an immunoperoxidase staining (ABC kit, Fisher). The intensity of immunohistochemical marker in the epidermis is evaluated relative to the negative control of the solvent (Water or DMSO 0.3%).

“Enriched in peptides having a molecular weight of less than 10 KD” as applied to a composition of the invention means that the dry weight % of peptides in the composition having a molecular weight of less than 10 KD is greater than the dry weight % of polypeptide/protein in the composition having a molecular weight of 10 KD or greater.

“Homolog” of a reference protein should be understood to mean a protein from a different species of plant having at least 60% sequence homology with the reference protein. Thus, for example, homologs of pea protein P13918 include:
>gi|137584|sp|P08438.1|VCL_VICFA RecName: Full=Vicilin; Flags: Precursor [Vicia faba]
>gi|22057|emb|CAA68559.1|vicilin [Vicia faba var. minor] >gi|383931031|gb|AFH56916.1|vicilin [Vicia faba]
>gi|502105533|ref|XP_004492829.1|PREDICTED: vicilin-like isoform X1 [Cicer arietinum] ChickPea
>gi|29539109|emb|CAD87730.1|allergen Len c 1.0101 [Lens culinaris] Lentil

A “variant” of an anti-inflammatory fragment shall be taken to mean a fragment having an amino acid sequence that is substantially identical to the anti-inflammatory fragment, and which has anti-inflammatory activity as defined above. Thus, for example, the term should be taken to include fragments that are altered in respect of one or more amino acid residues. Preferably such alterations involve the insertion, addition, deletion and/or substitution of 5 or fewer amino acids, more preferably of 4 or fewer, even more preferably of 3 or fewer, most preferably of 1 or 2 amino acids only. Insertion, addition and substitution with natural and modified amino acids is envisaged. The variant may have conservative amino acid changes, wherein the amino acid being introduced is similar structurally, chemically, or functionally to that being substituted. Generally, the variant will have at least 70% amino acid sequence homology, preferably at least 80% sequence homology, more preferably at least 90% sequence homology, and ideally at least 95%, 96%, 97%, 98% or 99% sequence homology with the parent anti-inflammatory fragment.

In this specification, the term “sequence identity” should be understand to comprise both sequence identity and similarity, i.e. a variant (or homolog) that shares 70% sequence identity with a reference sequence is one in which any 70% of aligned residues of the variant (or homolog) are identical to or conservative substitutions of the corresponding residues in the reference sequence across the entire length of the sequence. Sequence identity is the amount of characters which match exactly between two different sequences. Hereby, gaps are not counted and the measurement is relational to the shorter of the two sequences. In terms of “sequence homology”, the term should be understood to mean that a variant (or homolog) which shares a defined percent similarity or identity with a reference sequence when the percentage of aligned residues of the variant (or homolog) are either identical to, or conservative substitutions of, the corresponding residues in the reference sequence and where the variant (or homolog) shares the same function as the reference sequence. This alignment and the percent homology or sequence identity can be determined using software programs known in the art, for example, one alignment program is BLAST, using default parameters. Details of these programs can be found at the following Internet address: <www.ncbi.nlm.nih.gov/blast/Blast.cgi>.

### Variants of SEQ ID NO: 109 (Anti-Inflammatory Peptide (I_37)

Variants of SEQ ID NO: 109 (RGPQQYAEWQINEK) including variants having 1,2 or 3 conservative amino acid substitutions, 1, 2 to 3 non-conservative amino acid substitutions, 1-2 amino acid additions, 1, 2 or 3 amino acid deletions, are provided below:

One conservative amino acid substitution:

(SEQ ID NO: 268)
RGPQQYAEWQINER,;
(SEQ ID NO: 269)
RGPQQYAEWQINDK,;
(SEQ ID NO: 270)
RGPQQFAEWQINEK,;
(SEQ ID NO: 271)
KGPQQYAEWQINEK,,;
(SEQ ID NO: 272)
RGPEQYAEWQINEK,,;
(SEQ ID NO: 273)
RGPQEYAEWQINEK,,;
(SEQ ID NO: 274)
RGPQQYADWQINEK,,;
(SEQ ID NO: 275)
RGPQQYAEYQINEK,.

Two conservative amino acid substitutions:

(SEQ ID NO: 276)
KGPEQYAEWQINEK,;
(SEQ ID NO: 277)
KGPQEYAEWQINEK,;
(SEQ ID NO: 278)
KGPQQFAEWQINEK,;
(SEQ ID NO: 279)
RGPEQFAEWQINEK,;
(SEQ ID NO: 280)
KGPQQYAEWQINER,;
(SEQ ID NO: 281)
RGPQQYAEWQINDR,;
(SEQ ID NO: 282)
RGPQQYADWQINDK,;
(SEQ ID NO: 283)
RGPQQFAEWQINER,.

Three conservative amino acid substitutions:

(SEQ ID NO: 284)
RGPQQYAEWQVNEK,;
(SEQ ID NO: 285)
RGPQQFAEWQINEK,;
(SEQ ID NO: 286)
KGPQQFAEWQINER,;
(SEQ ID NO: 287)
KGPQQFAEWQVNEK,;
(SEQ ID NO: 288)
RGPQQFAEWQVNDK,;
(SEQ ID NO: 289)
RGPQQYADWQINDR,;
(SEQ ID NO: 290)
KGPQQYADWQINDK,;
(SEQ ID NO: 291)
RGPQQFADYQINEK,.

One non-conservative amino acid substitution

(SEQ ID NO: 292)
RGPQQYARWQINEK,;
(SEQ ID NO: 293)
RGPQQYAEWQINEE,;
(SEQ ID NO: 294)
HGPQQYAEWQINEK,,;
(SEQ ID NO: 295)
RGPYQYAEWQINEK,;
(SEQ ID NO: 296)
RGPQQYMEWQINEK,;
(SEQ ID NO: 297)
RGPQQYAEWCINEK,;
(SEQ ID NO: 292)
RGPQPYAEWQINEK,.

Two non-conservative amino acid substitution

(SEQ ID NO: 299)
RGGQQYAEWQINED,;
(SEQ ID NO: 300)
RGPQQYARWKINEK,;
(SEQ ID NO: 301)
RGGQQYAETQINEK,;
(SEQ ID NO: 302)
RGPLQYAEWQNNEK,;
(SEQ ID NO: 303)
EGPQQYAEWQINED,;
(SEQ ID NO: 304)
RGPQQYAEWQINLL,;
(SEQ ID NO: 305)
RGPQQGGEWQINEK,.

Three non-conservative amino acid substitution

(SEQ ID NO: 306)
RGPQQYAEWQIGGG,;
(SEQ ID NO: 307)
RGPQQKYEWQINEK,;
(SEQ ID NO: 308)
RGPQAQYEWQINEK,;
(SEQ ID NO: 309)
RPHQQYAEWQINEK,;
(SEQ ID NO: 310)
RGPQHHHEWQINEK,;
(SEQ ID NO: 311)
RGPPQYAPPQINEK,;
(SEQ ID NO: 312)
RGPQCYYEWCINEK,;
(SEQ ID NO: 313)
RGPTQYAEGQINEG,.

One or two amino acid additions

(SEQ ID NO: 314)
RGPQQYAEWQINEKG,;
(SEQ ID NO: 315)
RGPQQYAEWQINEKY,;
(SEQ ID NO: 316)
RGPQQYAFTEWQINEK,;
(SEQ ID NO: 317)
RGPQSQYAEWQINEKPM,;
(SEQ ID NO: 318)
RGPQQYAEWQINEKKK,;
(SEQ ID NO: 319)
RRRRGPQQYAEWQINEK.

The term “variant” typically encompasses fragments of the peptides of the invention. “Fragment of a peptide of the invention” or “peptide fragment” means a fragment of one of the peptides of the invention having at least 3, 4, 5, 6, 7, 8, 9, 10, 11, 12, 13, 14, 15, 16, 17, 18, 19, 20, 21 or 22 amino acids and that typically has a bioactivity, for example anti-inflammatory activity, cellular growth or proliferation promotion activity, glucose transport promoting activity, or anti-bacterial activity. In one embodiment, the fragment consists of at least 30%, 40%, 50%, 60%, 70%, 80%, or 90% of the reference sequence. Examples of fragments of the invention are provided in SEQ ID NOS 320-330.

(SEQ ID NO: 320)
RGPQQYAEWQINE,;
(SEQ ID NO: 321)
RGPQQYAEWQIN,;
(SEQ ID NO: 322)
RGPQQYAEWQI,;
(SEQ ID NO: 323)
GPQQYAEWQINEK,;
(SEQ ID NO: 324)
PQQYAEWQINEK,;
(SEQ ID NO: 325)
QQYAEWQINEK,;
(SEQ ID NO: 326)
QQYAEWQI,;
(SEQ ID NO: 327)
PQQYAEWQINE,;
(SEQ ID NO: 328)
PQQYAEWQIN,;
(SEQ ID NO: 329)
RGPQQYA,;
(SEQ ID NO: 330)
EWQINEK.

“Inflammatory disorder” means an immune-mediated inflammatory condition that affects humans and is generally characterised by dysregulated expression of one or more cytokines. Examples of inflammatory disorders include skin inflammatory disorders, inflammatory disorders of the joints, inflammatory disorders of the cardiovascular system, certain autoimmune diseases, lung and airway inflammatory disorders, intestinal inflammatory disorders. Examples of skin inflammatory disorders include dermatitis, for example atopic dermatitis and contact dermatitis, acne vulgaris, and psoriasis. Examples of inflammatory disorders of the joints include rheumatoid arthritis. Examples of inflammatory disorders of the cardiovascular system are cardiovascular disease and atherosclerosis. Examples of autoimmune diseases include Type 1 diabetes, Graves disease, Guillain-Barre disease, Lupus, Psoriatic arthritis, and Ulcerative colitis. Examples of lung and airway inflammatory disorders include asthma, cystic fibrosis, COPD, emphysema, and acute respiratory distress syndrome. Examples of intestinal inflammatory disorders include colitis and inflammatory bowel disease. Other inflammatory disorders include cancer, hay fever, periodontitis, allergies, hypersensitivity, ischemia, depression, systemic diseases, post infection inflammation and bronchitis.

In this specification, the term “Metabolic disorder” should be understood to include pre-diabetes, diabetes; Type-1 diabetes; Type-2 diabetes; metabolic syndrome; obesity; diabetic dyslipidemia; hyperlipidemia; hypertension; hypertriglyceridemia; hyperfattyacidemia; hypercholerterolemia; hyperinsulinemia, and MODY.

“Man-made” as applied to comestible products should be understood to mean made by a human being and not existing in nature.

“Maintaining or restoring gut health”: means reducing and/or regulating the pro-inflammatory response in the gut and more specifically the epithelial cells. The healthy microbiome offers some protection against pathogenic viruses and bacteria, and their presence is needed to guide the development of our immune system. It has been shown that these bacteria can react to human signals of stress, sickness, or age which can be manifested by inflammation and as a consequence switch on their virulence genes and cause or contribute to disease. Having the ability to reduce and maintain at healthy levels the inflammatory response can help maintain the healthy bacteria. Digestive problems, which comprise the number one health problem in North America, appear to be occurring with more frequency in recent years. One way to maintain digestive health is to maintain proper inflammation and intestinal flora.

“Maintaining or restoring muscle health” means helping retain or restore mammalian muscle health resulting from damage incurred during exercise. By lowering inflammation the peptides promote recovery from injuries during exercise, and relieve muscle soreness/pain and injury connected with exercise. They can also be used to decrease and prevent muscle cramping, and to allow a faster recovery from muscle cramping. Cramping can result from physical stress, mental stress, and or Repetitive Strain Injury stress. By lowering inflammation the peptides help reduce Myopathy of the muscle, and help prevent Sarcopenia in mammals, promote recovery from injuries during exercise, and relieve muscle soreness/pain and injury connected with exercise. They can also be used to decrease and prevent muscle cramping, and to allow a faster recovery from muscle cramping. Cramping can result from physical stress, mental stress, and or Repetitive Strain Injury stress. By lowering inflammation the peptides help reduce Myopathy of the muscle, and help prevent Sarcopenia in mammals.

In this specification, the term “composition” should be understood to mean something made by the hand of man, and not excludes naturally occurring compositions. Exemplary compositions include foods, beverages, nutritional supplements, personal care compositions, and pharmaceutical compositions.

In this specification, the term “substantially all” as applied to a list of peptides or fragments should be understood to mean at least 60%, 70%, 80%, 90% or 95% of the peptides or fragments.

In this specification, the term “personal care composition” should be understood to mean a composition formulated for use by humans in cleaning or treating the human body, particularly the skin, teeth, nails, feet and hair. Examples include shampoo, conditioner, skin creams and lotions, powders, dentrifice, shower gel or creams, body lotion, deodorant, and anti-perspirant.

In this specification, the term “nutritional supplement” should be understood to mean a product formulated for ingestion by a mammal and intended to confer a health benefit on the recipient. The supplement can take any form, for example a solid, liquid, or powder. Examples of supplements include powders, tablets, capsules, and drinks.

### BRIEF DESCRIPTION OF THE FIGURES

FIGS. 1 to 18: The effect of eighteen synthetic peptides of the invention on TNF-secretion in THP1 cells. All experiments were prepared in duplicate on three plates (6 wells/conditions). Significance was calculated using Students t-test (*p<0.05 compared to control, **p<0.01 compared to control, ***p<0.001 compared to control). The figures correspond with the following SEQ ID Nos: FIG. 1 is SEQ ID NO: 339, FIG. 2 is SEQ ID NO: 352, FIG. 3 is SEQ ID NO: 341, FIG. 4 is SEQ ID NO: 351, FIG. 5 is SEQ ID NO: 144, FIG. 6 is SEQ ID NO: 93, FIG. 7 is SEQ ID NO: 320, FIG. 8 is SEQ ID NO: 92, FIG. 9 is SEQ ID NO: 75, FIG. 10 is SEQ ID NO: 76, FIG. 11 is SEQ ID NO: 349, FIG. 12 is SEQ ID NO: 350, FIG. 13 is SEQ ID NO: 105, FIG. 14 is SEQ ID NO: 177, FIG. 15 is SEQ ID NO: 345, FIG. 16 is SEQ ID NO: 353, FIG. 17 is SEQ ID NO: 344, FIG. 18 is SEQ ID NO: 346.

FIG. 19. Viability of J774.2 macrophages after treatment with synthetic peptides. J774.2 macrophages were treated with 100 μM of synthetic peptide for 24 hours before an alamar blue assay was performed. Data are presented as an average of n=3+/−SEM.

FIGS. 20A-20B. The effects of peptide compositions on cell survival. J774.2 macrophages were treated with (FIG. 20A) 1 mg/ml or (FIG. 20B) 0.5 mg/ml of peptide compositions for 24 hours before an alamar blue assay was performed. Data is shown as (FIG. 20A) n=1+/−SEM and (FIG. 20B) n=3+/−SEM.

FIGS. 21A-21B. The effect of DMSO vehicle on TNFα and IL-1β secretion from J774.2 macrophages. J774.2 macrophages were treated with a final concentration of 0.3% and 1% DMSO (equivalent to the amounts used to dissolve the peptides) for 24 hours and the effect on TNFα (FIG. 21A) and IL-1β (FIG. 21B) after stimulation was established. Data are presented as an average of n=3+/−SEM. (***p<0.001 w.r.t LPS).

FIGS. 22A-22B. The effect of six peptides of the invention on TNFα and IL-1β secretion from J774.2 macrophages. J774.2 macrophages were treated with 100 μM of synthetic peptide for 24 hours and then stimulated with (FIG. 22A) LPS (10 ng/ml) for five hours or (FIG. 22B) LPS (10 ng/ml) for 5 hours followed by ATP (5 mM) for one hour. Supernatant was collected and levels of (FIG. 22A) TNFα and (FIG. 22B) IL-1β were determined by ELISA. (***p<0.001 w.r.t LPS, **p<0.01 w.r.t LPS, *p<0.05, ###p<0.001 w.r.t. LPS/ATP, ##p<0.01 w.r.t LPS/ATP and #p<0.05 w.r.t LPS/ATP). Final concentration of DMSO in well: SP1—0.3%, SP2—0%, SP3—0.3%, SP4—1%, SP5—1%, SP6—0.3%, Positive Control—0%. Data are presented as an average of n=3+/−SEM.

FIGS. 23A-23B. The effect a peptide composition of the invention on TNFα and IL-1β secretion. J774.2 macrophages were treated with 0.5 mg/ml of peptide composition for 24 hours and then stimulated with (FIG. 23A) LPS (10 ng/ml) for five hours or (FIG. 23B) LPS (10 ng/ml) for 5 hours followed by ATP (5 mM) for one hour. Supernatant was collected and levels of (FIG. 23A) TNFα and (FIG. 23B) IL-1β were determined by ELISA. (***p<0.001 w.r.t untreated+LPS, ###p<0.001 w.r.t. untreated+LPS/ATP). Data are presented as an average of n=3+/−SEM.

FIGS. 24A-24B. The effects of synthetic peptides with DMSO vehicle on TNFα J774.2 macrophages were treated with 100 μM of synthetic peptide SP1, SP3 and SP6 (FIG. 24A) or SP4 and SP5 (FIG. 24B) for 24 hours and then LPS (10 ng/ml) for five hours. Supernatant was collected and levels of TNFα were determined by ELISA. ###p<0.001 w.r.t 0.3% DMSO+LPS, ##p<0.01 w.r.t. 0.3% DMSO+LPS, +++p<0.001 w.r.t 1% DMSO+LPS, ++p<0.01 w.r.t 1% DMSO+LPS/ATP). Final concentration of DMSO in well: positive control—0% , SP1—0.3%, SP2—0%, SP3—0.3%, SP4—1%, SP5—1%, SP6—0.3%.

FIG. 25. THP-1 differentiated macrophages treated with a composition of rice peptides of the invention (I_2_HR) for 24 hrs. prior to LPS stimulation were compared to untreated cells. TNF-α secretion in I_2_HR treated cells is reduced by 92% vs. untreated cells. Significant results are observed at 100 ug/ml and 500 ug/ml concentrations of I_2_HR, indicating the potency of I_2_HR.

FIGS. 26 to 28. The effect of three synthetic peptides of the invention on TNF-secretion in THP1 cells. All experiments were prepared in duplicate on three plates (6 wells/conditions). Significance was calculated using Students t-test (*p<0.05 compared to control, **p<0.01 compared to control, ***p<0.001 compared to control). The figures correspond with the following SEQ ID Nos: FIG. 26 is SEQ ID NO: 85, FIG. 27 is SEQ ID NO: 91, FIG. 28 is SEQ ID NO: 420.

### DETAILED DESCRIPTION OF THE INVENTION

### Example 1—Inflammatory Response

TNF-α is secreted by macrophages in response to stimulation by endotoxins such as lipopolysaccharides (LPS). TNF-α is thought to be involved in systemic inflammation and dysregulation of TNF-α production is thought to be involved in many diseases. The Biolegend assay is a sandwich ELISA kit that is designed for the accurate quantitation of human TNF-α from cell culture supernatant, serum or plasma.

THP-1 monocytes were seeded in a 96 well plate at 10,000 cells per well in RPMI containing 10% fetal calf serum (FCS), 1% Pen/strep, 1% L-glutamine, 100 nM PMA and allowed to differentiated for 72 h prior to experimentation.

Following differentiation the cells were incubated with 100 ng/ml, 10 ng/ml or 1 ng/ml synthetic peptide for 24 h respectively.

Following treatment the cells were stimulated with 10 ng/ml LPS for 5 h and the quantity of TNF-α in the supernatant determined using the Biolegend assay ELISA kit.

Results were calculated as a percentage of the untreated control. An increase in optical density reading indicates greater quantity of TNF-α release into cell culture supernatant.

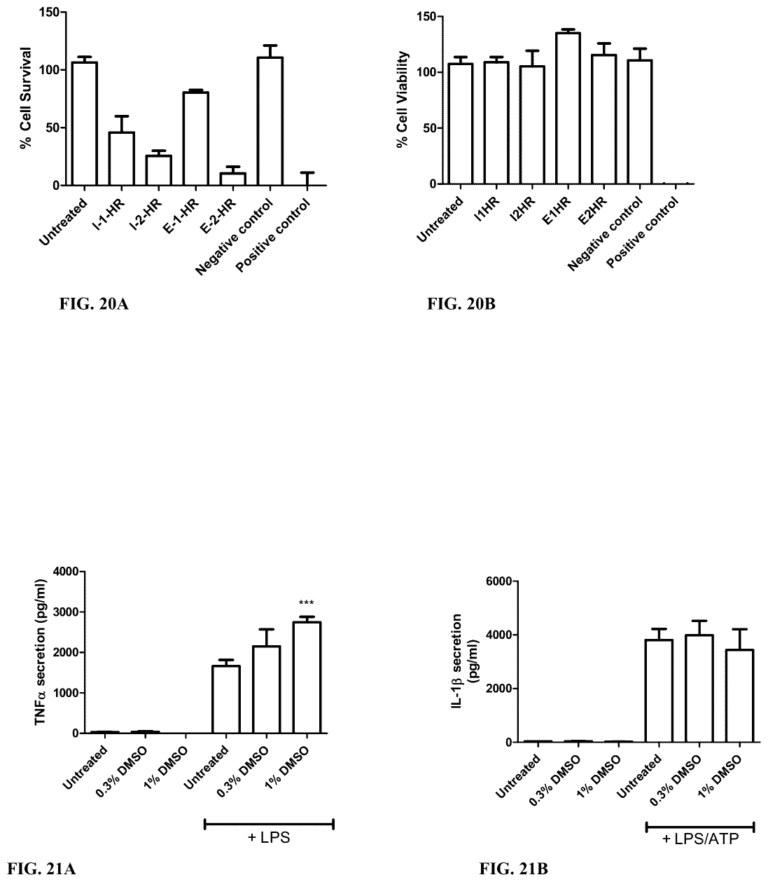

The results are provided in FIGS. 1 to 21 and summarised in Table 1 below. All experiments were prepared in duplicate on three plates (6 wells/conditions). Significance was calculated using Students t-test (*p<0.05 compared to control, **p<0.01 compared to control, ***p<0.001 compared to control).

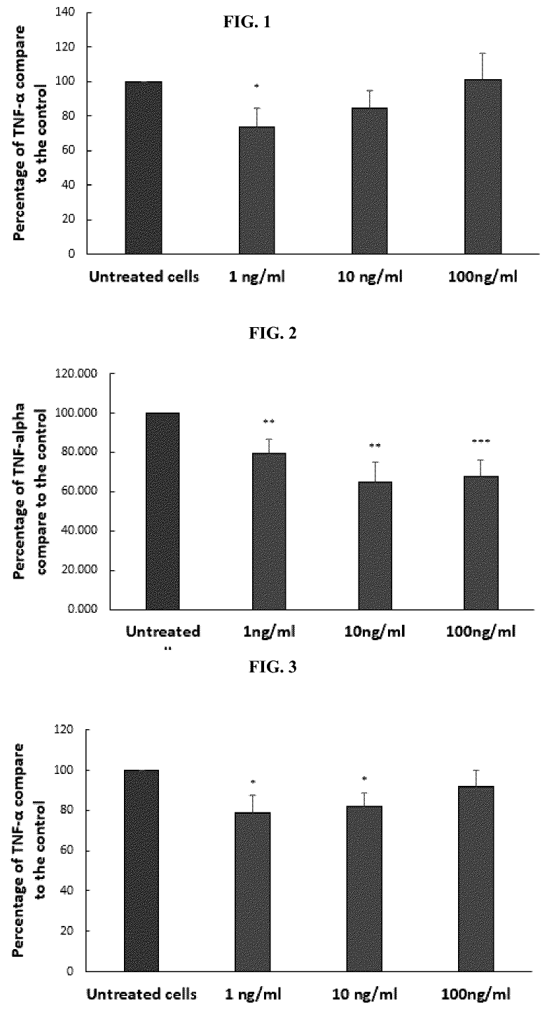

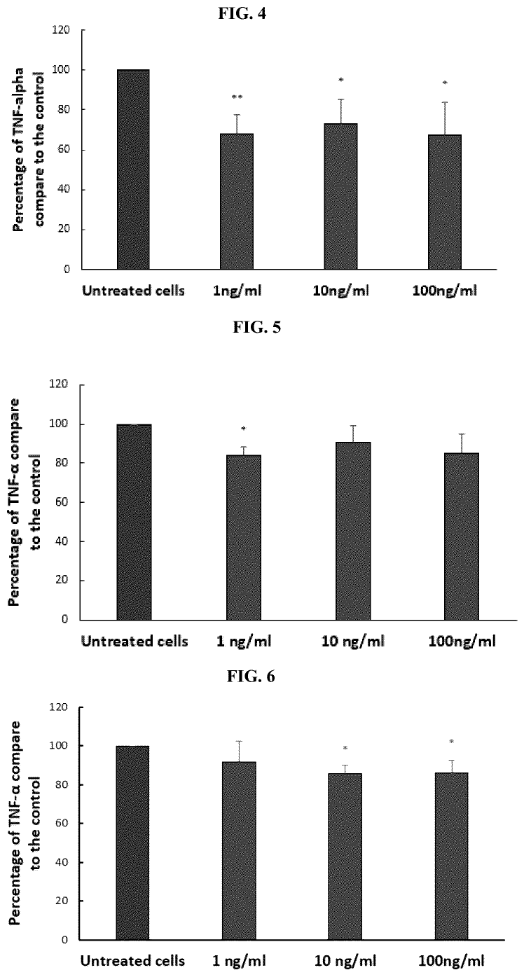

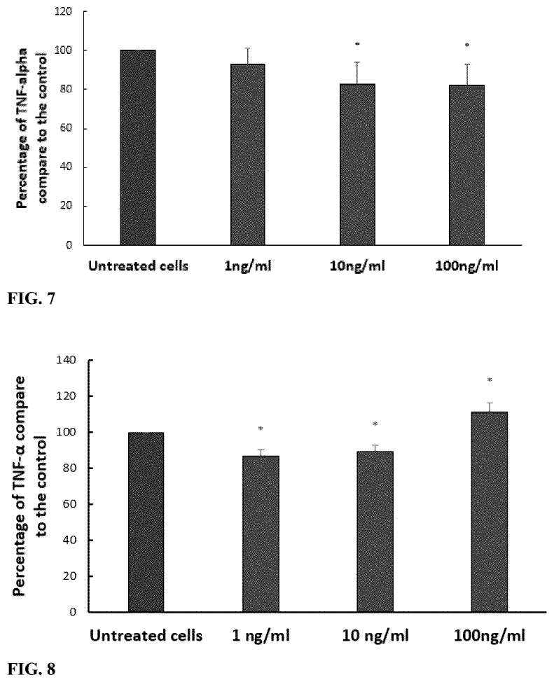

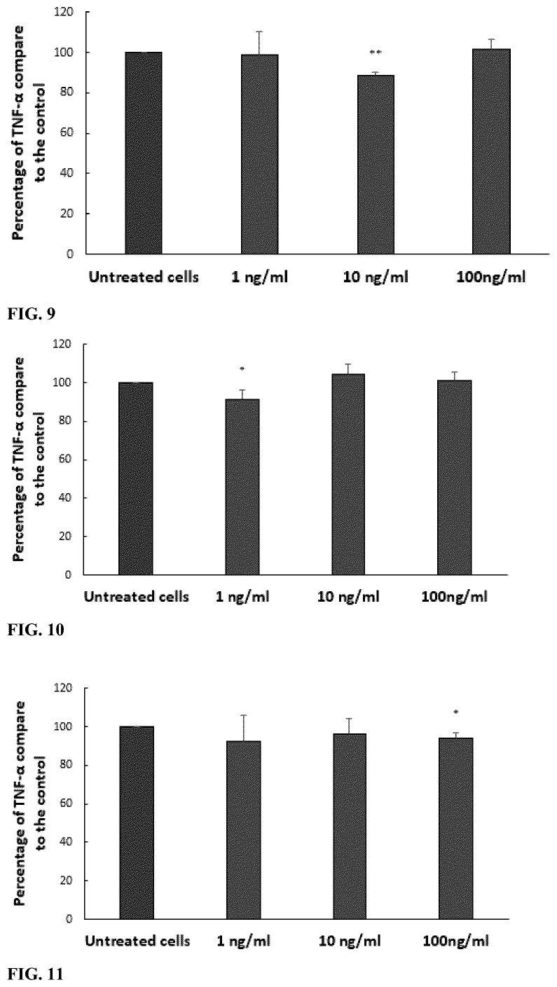

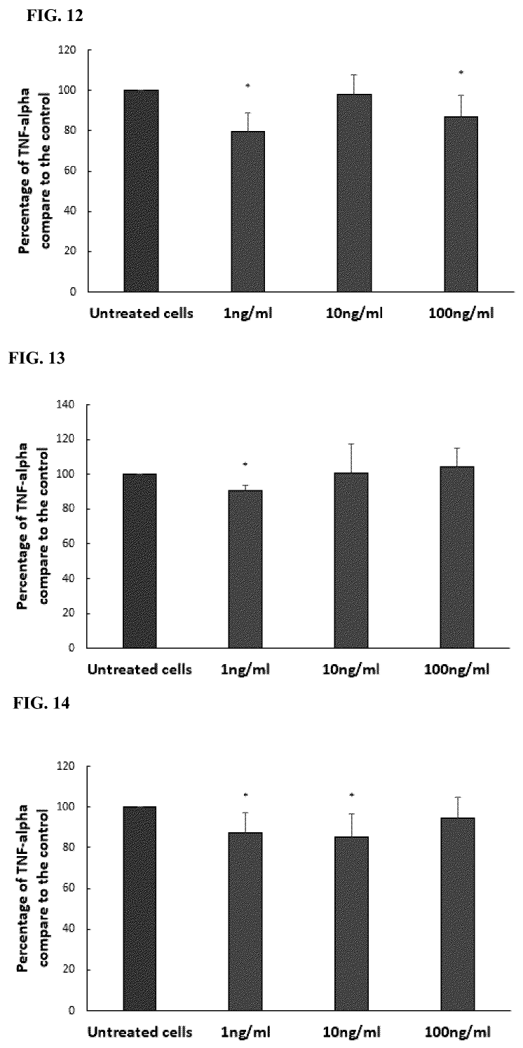

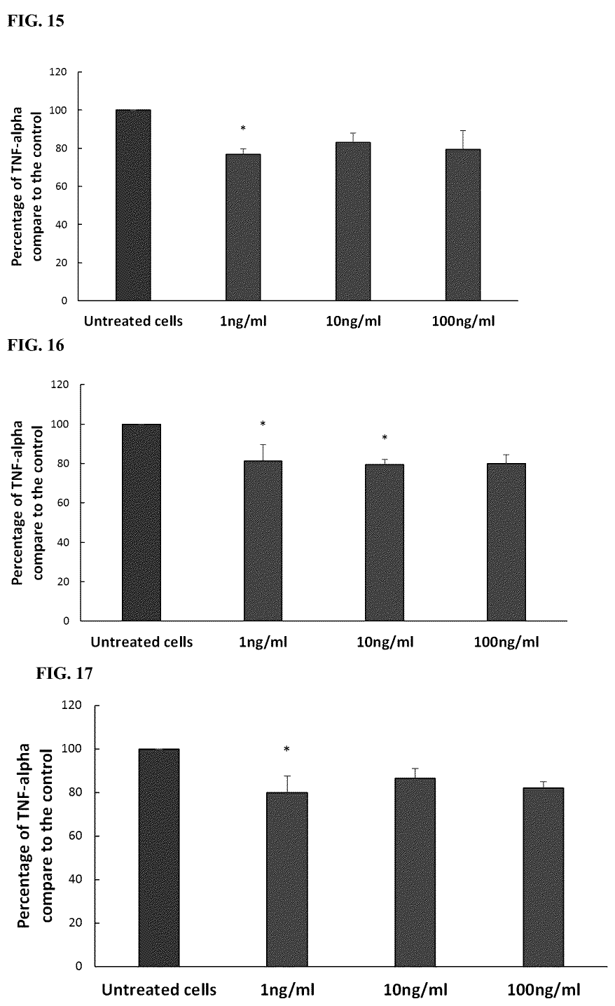

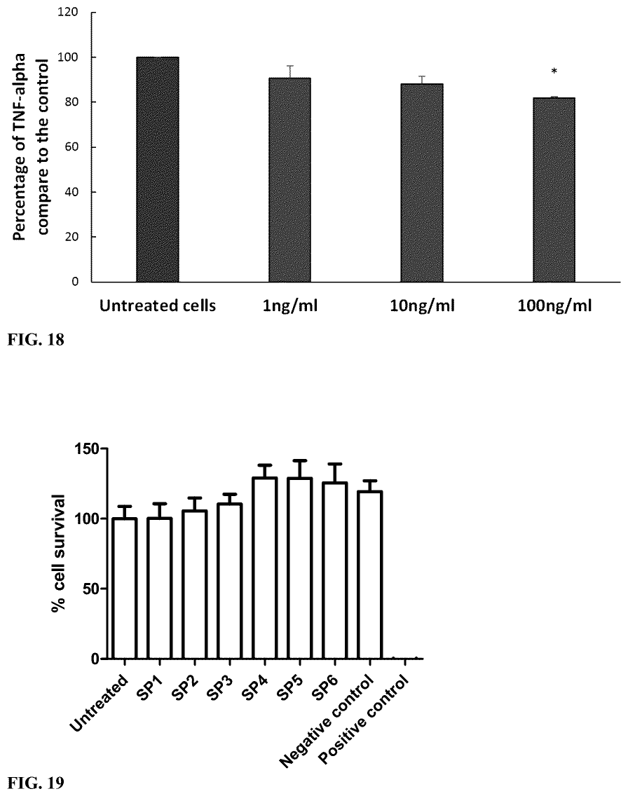

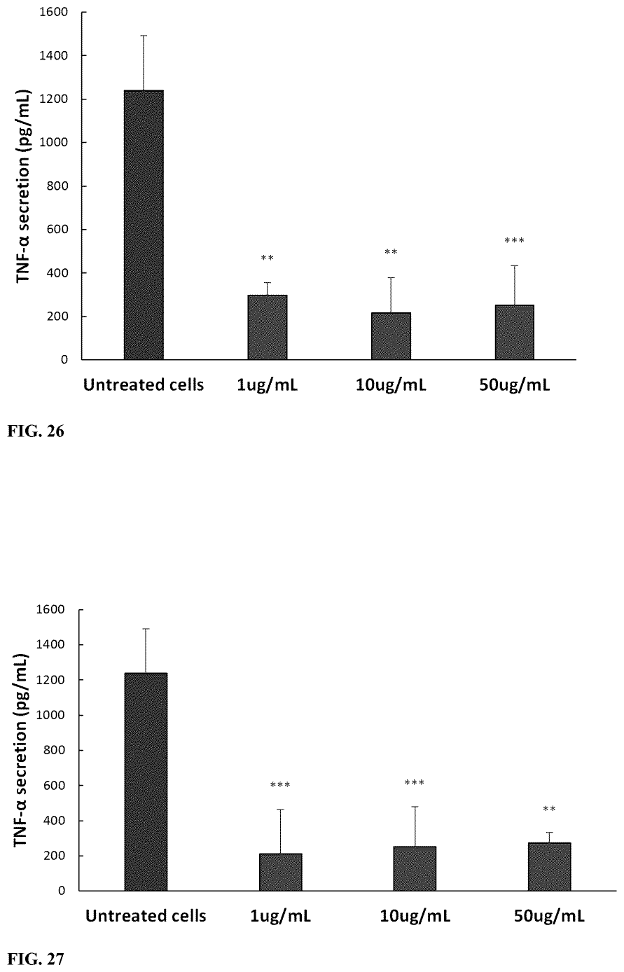

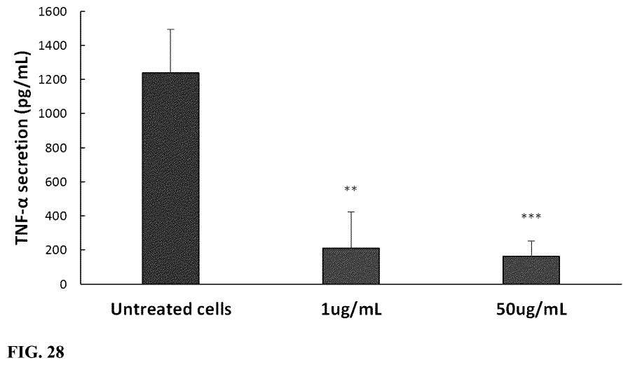

TABLE 1

| FIG. NUMBER | SEQ ID | TNF-α DECREASE |
|---|---|---|
| FIG. 1 | 339 | 26% |
| FIG. 2 | 352 | 23% |
| FIG. 3 | 341 | 21% |
| FIG. 4 | 351 | 18% |
| FIG. 5 | 144 | 16% |
| FIG. 6 | 93 | 14% |
| FIG. 7 | 320 | 13% |
| FIG. 8 | 92 | 13% |
| FIG. 9 | 75 | 11% |
| FIG. 10 | 76 | 9% |
| FIG. 11 | 349 | 6% |
| FIG. 12 | 350 | |
| FIG. 13 | 105 | 9% |
| FIG. 14 | 177 | |
| FIG. 15 | 345 | 23% |
| FIG. 16 | 353 | 20% |
| FIG. 17 | 344 | 20% |
| FIG. 18 | 346 | 18% |
| FIG. 26 | 85 | 80% |
| FIG. 27 | 91 | 80% |
| FIG. 28 | 420 | 80% |

### Example 2—Inflammatory Response

The effect of six synthetic peptides of the invention, SP1 to SP6 (SEQ ID NOs: 108, 109, 110, 111, 85 and 91) and four peptide compositions on the inflammatory response in vitro using a cell line was determined.

Peptide composition I_1_HR (Rice) contained the followings peptides (identified by SEQ ID NOs:) 116, 197, 207, 112, 211, 158, 201, 203, 114, 183, 130, 113, 182, 167, 166, 152, 220, 213, 215, 154, 219, 218, 165, 123, 185, 190, 209, 181, 198, 200, 147, 172, 184, 124, 153, 205, 115, 196, 151, 161, 160, 216, 210, 208, 146, 133, 204, 212, and 206).

Peptide composition I_2_HR (Rice) contained the followings peptides (identified by SEQ ID NOs:) 189, 177, 174, 129, 176, 202, 193, 195, 194, 192, 182, 128, 220, 127, 134, 136, 135, 180, 179, 178, 219, 218, 145, 120, 175, 190, 149, 126, 187, 191, 121, 122, 159, 132, 162, 137, 150, 186, 188, 164, 118, 125, 163, 157, 156, and 117.

Peptide composition E_1_HR (Pea) contained the followings peptides (identified by by SEQ ID NOs:) 74, 76, 106, 102, 101, 100, 92, 96, 83, 89, 90, 104, 82, 75, 79, 78, 77, 99, 103, 72, 86, 105, 94, 93, 81, 97, 80, 88, 85, 87, 71, 107, 73, 84, 98, and 95.

Peptide composition E_2_HR contained homologs of the peptides of the invention.

A J774.2 mouse macrophage cell line was treated with 100 μM of each synthetic peptide (SP) and 0.5 mg/ml of each peptide composition and the effect on two pro-inflammatory markers—tumour necrosis factor α (TNFα) and interleukin-1β (IL-1β) was determined after inflammation was induced using lipopolysaccharide (LPS) as an inflammatory stimulus. A one way anova was used with the dunnett test which is a multiple comparison and compares every mean with a single control mean.

### Example 3—Synthetic Peptides: Cell Viability

Synthetic peptides were first diluted in a suitable solvent. Dimethyl sulfoxide (DMSO) was the solvent of choice for peptides with poor predicted water solubility. Final concentration of DMSO in each well: SP1 (1_155_HR)—0.3%, SP2 (1_374_HR)—0%, SP3 (E_155_HR)—0.3%, SP4 (E_54_HR)—1%, SP5 (E_41_HR)—1%, SP6 (E_788_HR)—0.3%, positive Control—0%. Cells were first treated with 100 μM of each SP for 24 hours before an alamar blue assay was performed. No viability issues were seen with any of the peptides.

### Example 4—Peptide Compositions: Preparation and Toxicity

The peptide compositions were prepared by adjusting the pH to between 6-7 and sterile filtering. The effects of the peptide compositions on cell viability was determined. J774.2 macrophages were treated with 1 mg/ml and 0.5 mg/ml of each peptide composition, hydrogen peroxide to induce cell death as a positive control, and a peptide known to be non-toxic as a negative control. An alamar blue assay was then performed and cell survival is shown in FIG. 19 as a percentage of untreated (100%). As cell survival was compromised with 1 mg/ml of peptide, 0.5 mg/ml of peptide composition was used for further assays.

### Example 5—Inflammatory Markers

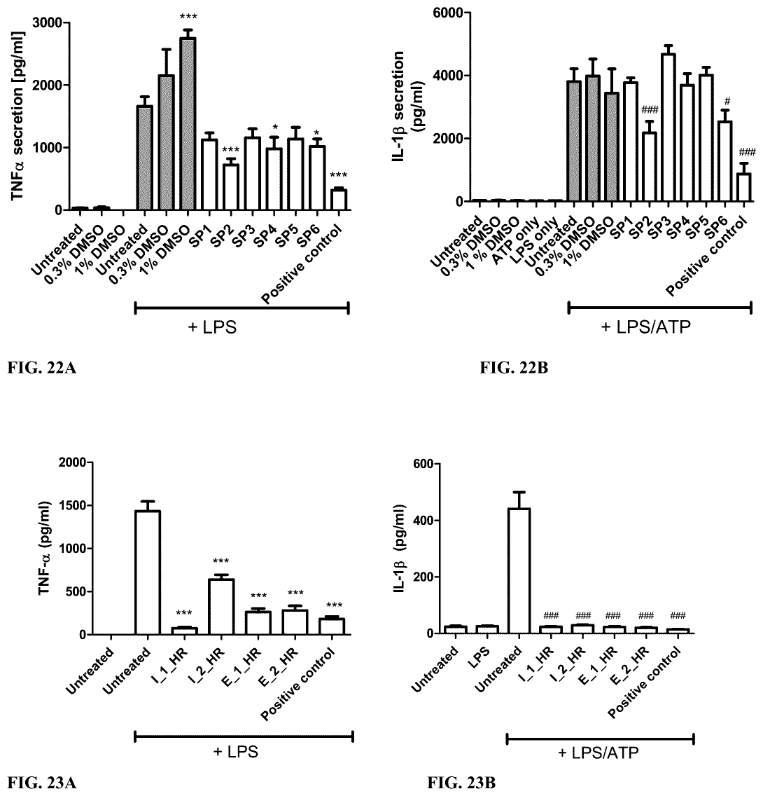

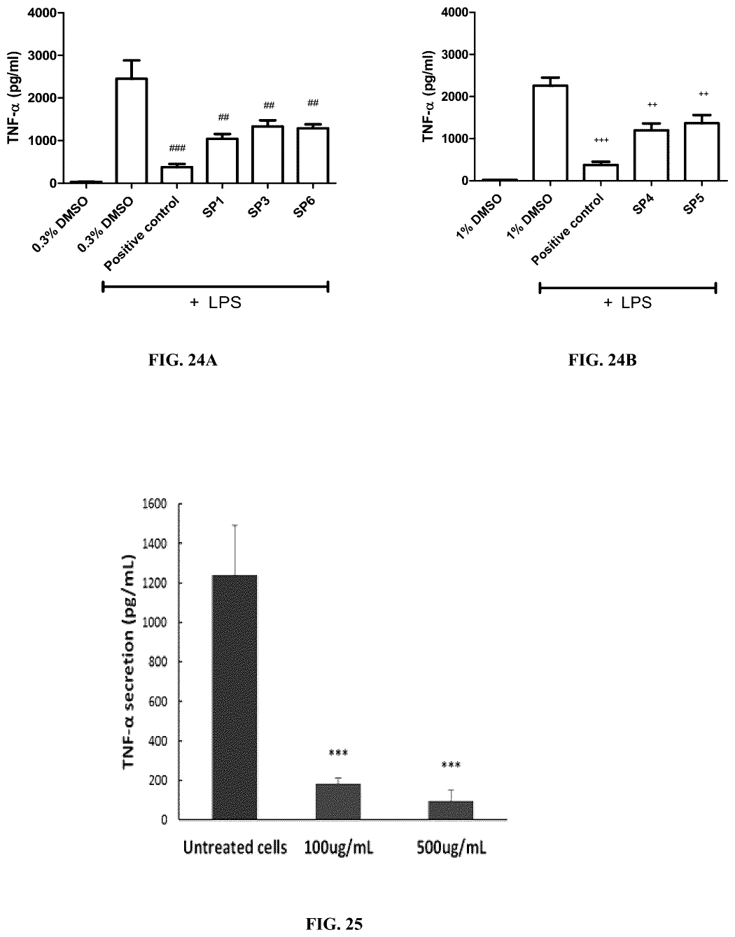

The effect of the DMSO on TNFα and IL-1β secretion was determined. 1% DMSO significantly increased levels of TNFα (FIG. 21A. ***p<0.001 w.r.t LPS) and this was taken into account when analysing the effect of the peptides on TNFα. No significant effect was seen with regards DMSO and IL-1β secretion.

### Example 6—Inflammatory Response

THP-1 differentiated macrophages were treated with a composition of rice peptides of the invention (I_2_HR) for 24 hrs. prior to LPS stimulation were compared to untreated cells. TNF-α secretion in I_2_HR treated cells is reduced by 92% vs. untreated cells. Significant results are observed at 100 ug/ml and 500 ug/ml concentrations of I_2_HR, indicating the potency of I_2_HR.

The invention is not limited to the embodiments hereinbefore described which may be varied in construction and detail without departing from the spirit of the invention.

### Sequence Information

SEQUENCE INFORMATION

PEA PROTEIN 1: P13918-1-
MAATTMKASFPLLMLMGISFLASVCVSSRSDPQNPFIFKSNKFQTLFENENGHIRLLQKFDQRSKIFENLQNYRLLEYKSKPHTIFLPQHTDA
DYILVVLSGKAILTVLKPDDRNSFNLERGDTIKLPAGTIAYLVNRDDNEELRVLDLAIPVNRPGQLQSFLLSGNQNQQNYLSGFSKNILEASFN
TDYEEIEKVLLEEHEKETQHRRSLKDKRQQSQEENVIVKLSRGQIEELSKNAKSTSKKSVSSESEPFNLRSRGPIYSNEFGKFFEITPEKNPQLQ
DLDIFVNSVEIKEGSLLLPHYNSRAIVIVTVNEGKGDFELVGQRNENQQEQRKEDDEEEEQGEEEINKQVQNYKAKLSSGDVFVIPAGHPVA
VKASSNLDLLGFGINAENNQRNFLAGDEDNVISQIQRPVKELAFPGSAQEVDRILENQKQSHFADAQPQQRERGSRETRDRLSSV [SEQ
ID 1]
REGION: 253 to 274-PFNLRSRGPIYSNEFGKFFEIT [SEQ ID 17]
PEPTIDE: SRGPIYSNEFGK [SEQ ID 71]
REGION: 110 to 126-KPDDRNSFNLERGDTIK [SEQ ID 18]
PEPTIDE: NSFNLER [SEQ ID 72]
REGION: 134 to 160-YLVNRDDNEELRVLDLAIPVNRPGQLQ [SEQ ID 19]
PEPTIDE: VLDLAIPVNR [SEQ ID 73]
PEPTIDE: DDNEELR [SEQ ID 74]
REGION: 331 to 381-QEQRKEDDEEEEQGEEEINKQVQNYKAKLSSGDVFVIPAGHPVAVKASSNL [SEQ ID 20]
PEPTIDE: LSSGDVFVIPAGHPVAVK [SEQ ID 75]
PEPTIDE: EDDEEEEQGEEEINK [SEQ ID 76]
REGION: 175 to 242-SGFSKNILEASFNTDYEEIEKVLLEEHEKETQHRRSLKDKRQQSQEENVIVKLSRGQIEELSKNAKST [SEQ
ID 21]
PEPTIDE: NILEASFNTDYEEIEKVLLEEHEKETQHR [SEQ ID 77]
PEPTIDE: NILEASFNTDYEEIEKVLLEEHEK [SEQ ID 78]
PEPTIDE: NILEASFNTDYEEIEK [SEQ ID 79]
PEPTIDE: RQQSQEENVIVK [SEQ ID 80]
PEPTIDE: QQSQEENVIVK [SEQ ID 81]
PEPTIDE: LSRGQIEELSK [SEQ ID 82]
PEPTIDE: GQIEELSK [SEQ ID 83]
PEPTIDE: VLLEEHEK [SEQ ID 84]
REGION: 35 to 108-PFIFKSNKFQTLFENENGHIRLLQKFDQRSKIFENLQNYRLLEYKSKPHTIFLPQHTDADYILVVLSGKAILTV
[SEQ ID 22]
PEPTIDE: SKPHTIFLPQHTDADYILVVLSGK [SEQ ID 85]
PEPTIDE: PHTIFLPQHTDADYILVVLSGK [SEQ ID 86]
PEPTIDE: SNKFQTLFENENGHIR [SEQ ID 87]
PEPTIDE: SKIFENLQNYR [SEQ ID 88]
PEPTIDE: IFENLQNYR [SEQ ID 89]
REGION: 423 to 450-QEVDRILENQKQSHFADAQPQQRERGSR [SEQ ID 23]
PEPTIDE: ILENQKQSHFADAQPQQR [SEQ ID 90]
PEPTIDE PGQLQSFLLSGNQNQQNYLSGFSK [SEQ ID 91]
PEA PROTEIN 2: P02856-2-
DRRQELSNENVLVKVSRRQLEELSKNAKSSSRRSVSSESGPFNLRSEDPLYSNNSGKFFELTPEKNQQLQDLDLFVNSVDLKEGSLLLPNYNS
RALLVLVLVVNEGKGDFELVGQRNENQGKEN [SEQ ID 2]
REGION: 53 to 87-NNSGKFFELTPEKNQQLQDLDLFVNSVDLKEGSLL [SEQ ID 24]
PEPTIDE: FFELTPEKNQQLQDLDLFVNSVDLK [SEQ ID 92]
REGION: 14 to 30-KVSRRQLEELSKNAKSS [SEQ ID 25]
PEPTIDE: QLEELSK [SEQ ID 93]
PEA PROTEIN 3: P15838-3-
MATKLLALSLSFCFLLLGGCFALREQPEQNECQLERLNALEPDNRIESEGGLIETWNPNNKQFRCAGVALSRATLQHNALRRPYYSNAPQEI
FIQQGNGYFGMVFPGCPETFEEPQESEQGEGRRYRDRHQKVNRFREGDIIAVPTGIVFWMYNDQDTPVIAVSLTDIRSSNNQLDQMPRR
FYLAGNHEQEFLRYQHQQGGKQEQENEGNNIFSGFKRDFLEDAFNVNRHIVDRLQGRNEDEEKGAIVKVKGGLSIISPPEKQARHQRGSR
QEEDEDEDEERQPRHQRGSRQEEEEDEDEERQPRHQRRRGEEEEEDKKERRGSQKGKSRRQGDNGLEETVCTAKLRLNIGPSSSPDIYNPE
AGRIKTVTSLDLPVLRWLKLSAEHGSLHKNAMFVPHYNLNANSIIYALKGRARLQVVNCNGNTVFDGELEAGRALTVPQNYAVAAKSLSD
RFSYVAFKTNDRAGIARLAGTSSVINNLPLDVVAATFNLQRNEARQLKSNNPFKFLVPARQSENRASA [SEQ ID 3]
REGION: 267 to 287-QRGSRQEEDEDEDEERQPRHQ [SEQ ID 26]
PEPTIDE: QEEDEDEDEER [SEQ ID 94]
REGION: 190 to 222-QEFLRYQHQQGGKQEQENEGNNIFSGFKRDFLE [SEQ ID 27]
PEPTIDE: YQHQQGGKQEQENEGNNIFSGFK [SEQ ID 95]
PEA PROTEIN 4: Q9M3X6-4-
MATTIKSRFPLLLLLGIIFLASVVCVTYANYDEGSEPRVPAQRERGRQEGEKEEKRHGEWRPSYEKEEDEEEGQRERGRQEGEKEEKRHGE
WRPSYEKQEDEEEKQKYRYQREKEDEEEKQKYQYQREKKEQKEVQPGRERWEREEDEEQVDEEWRGSQRREDPEERARLRHREERTKR
DRRHQREGEEEERSSESQERRNPFLFKSNKFLTLFENENGHIRLLQRFDKRSDLFENLQNYRLVEYRAKPHTIFLPQHIDADLILVVLSGKAILT
VLSPNDRNSYNLERGDTIKLPAGTTSYLVNQDDEEDLRLVDLVIPVNGPGKFEAFDLAKNKNQYLRGFSKNILEASYNTRYETIEKVLLEEQE
KDRKRRQQGEETDAIVKVSREQIEELKKLAKSSSKKSLPSEFEPINLRSHKPEYSNKFGKLFEITPEKKYPQLQDLDLFVSCVEINEGALMLPHY
NSRAIVVLLVNEGKGNLELLGLKNEQQEREDRKERNNEVQRYEARLSPGDVVIIPAGHPVAITASSNLNLLGFGINAENNERNFLSGSDDNV
ISQIENPVKELTFPGSVQEINRLIKNQKQSHFANAEPEQKEQGSQGKRSPLSSILGTFY [SEQ ID 4]
REGION: 284 to 317-YNLERGDTIKLPAGTTSYLVNQDDEEDLRLVDLV [SEQ ID 28]
PEPTIDE: GDTIKLPAGTTSYLVNQDDEEDLR [SEQ ID 96]
REGION: 321 to 400-
NGPGKFEAFDLAKNKNQYLRGFSKNILEASYNTRYETIEKVLLEEQEKDRKRRQQGEETDAIVKVSREQIEELKKLAKSS [SEQ ID 29]
PEPTIDE: RQQGEETDAIVK [SEQ ID 97]
PEPTIDE: VLLEEQEKDRK [SEQ ID 98]
PEPTIDE: NILEASYNTR [SEQ ID 99]
PEPTIDE: FEAFDLAK [SEQ ID 100]
PEPTIDE: EQIEELKK [SEQ ID 101]
PEPTIDE: EQIEELK [SEQ ID 102]
PEPTIDE: NKNQYLR [SEQ ID 103]
REGION: 503 to 549-RYEARLSPGDVVIIPAGHPVAITASSNLNLLGFGINAENNERNFLSG [SEQ ID 30]
PEPTIDE: LSPGDVVIIPAGHPVAITASSNLNLLGFGINAENNER [SEQ ID 104]
REGION: 89 to 112-HGEWRPSYEKQEDEEEKQKYRYQR [SEQ ID 31]
PEPTIDE: PSYEKQEDEEEKQK [SEQ ID 105]
REGION: 62 to 80-PSYEKEEDEEEGQRERGRQ [SEQ ID 32]
PEPTIDE: EEDEEEGQR [SEQ ID 106]
PEA PROTEIN 5: D3VNE1-5-
MAATPIKPLMLLAIAFLASVCVSSRSDQENPFIFKSNRFQTLYENENGHIRLLQKFDKRSKIFENLQNYRLLEYKSKPRTLFLPQYTDADFILVV
LSGKATLTVLKSNDRNSFNLERGDTIKLPAGTIAYLANRDDNEDLRVLDLTIPVNKPGQLQSFLLSGTQNQPSLLSGFSKNILEAAFNTNYEEI
EKVLLEQQEQEPQHRRSLKDRRQEINEENVIVKVSREQIEELSKNAKSSSKKSVSSESGPFNLRSRNPIYSNKFGKFFEITPEKNQQLQDLDIF
VNSVDIKEGSLLLPNYNSRAIVIVTVTEGKGDFELVGQRNENQGKENDKEEEQEEETSKQVQLYRAKLSPGDVFVIPAGHPVAINASSDLNLI
GFGINAENNERNFLAGEEDNVISQVERPVKELAFPGSSHEVDRLLKNQKQSYFANAQPLQRE [SEQ ID 5]
REGION: 75 to 104-KSKPRTLFLPQYTDADFILVVLSGKATLTV [SEQ ID 33]
PEPTIDE: TLFLPQYTDADFILVVLSGK [SEQ ID 107]
RICE PROTEIN 7: Q6K508
MATTTSLLSSCLCALLLAPLFSQGVDAWESRQGASRQCRFDRLQAFEPLRKVRSEAGDTE
YFDERNEQFRCAGVFVIRRVIEPQGLVVPRYSNTPALAYIIQGKGYVGLTFPGCPATHQQ
QFQLFEQRQSDQAHKFRDEHQKIHEFRQGDVVALPASVAHWFYNGGDTPAVVVYVYDIKS
FANQLEPRQKEFLLAGNNQRGQQIFEHSIFQHSGQNIFSGFNTEVLSEALGINTEASKRL
QSQNDQRGDIIRVKHGLQLLKPTLTQRQEEHRQYQQVQYREGQYNGLDENFCTIKARVNI
ENPSRADYYNPRAGRITLLNNQKFPILNLIGMGAARVNLYQNALLSPFWNINAHSVVYII
QGSVRVQVANNQGRSVFNGVLHQGQLLIIPQNHAVIKKAEHNGCQYVAIKTISDPTVSWV
AGKNSILRALPVDVIANAYRISRDEARRLKNNRADEIGPFTPRFPQKSQRGYQFLTEGLS
LIGM [SEQ ID 6]
PEPTIDE: GYVGLTFPGCPATHQQQFQLFEQR [SEQ ID 108]
RICE PROTEIN 8: Q6K7K6
MASMSTILPLCLGLLLFFQVSMAQFSFGGSPLQSPRGFRGDQDSRHQCRFEHLTALEATH
QQRSEAGFTEYYNIEARNEFRCAGVSVRRLVVESKGLVLPMYANAHKLVYIVQGRGVFGM
ALPGCPETFQSVRSPFEQEVATAGEAQSSIQKMRDEHQQLHQFHQGDVIAVPAGVAHWLY
NNGDSPVVAFTVIDTSNNANQLDPKRREFFLAGKPRSSWQQQSYSYQTEQLSRNQNIFAG
FSPDLLSEALSVSKQTVLRLQGLSDPRGAIIRVENGLQALQPSLQVEPVKEEQTQAYLPT
KQLQPTWLRSGGACGQQNVLDEIMCAFKLRKNIDNPQSSDIFNPHGGRITRANSQNFPIL
NIIQMSATRIVLQNNALLTPHWTVNAHTVMYVTAGQGHIQVVDHRGRSVFDGELHQQQIL
LIPQNFAVVVKARREGFAWVSFKTNHNAVDSQIAGKASILRALPVDVVANAYRLSREDSR
HVKFNRGDEMAVFAPRRGPQQYAEWQINEK [SEQ ID 7]
PEPTIDE: RGPQQYAEWQINEK [SEQ ID 109]
PEA PROTEIN 6: P13919
MATTIKSRFPLLLLLGIIFLASVVSVTYANYDEGSEPRVPAQRERGRQEGEKEEKRHGEW
RPSYEKEEDEEEGQRERGRQEGEKEEKRHGEWGPSYEKQEDEEEKQKYRYQREKEDEEEK
QKYQYQREKKEQKEVQPGRERWEREEDEEQVDEEWRGSQRREDPEERARLRHREERTKRD
RRHQREGEEEERSSESQERRNPFLFKSNKFLTLFENENGHIRLLQRFDKRSDLFENLQNY
RLVEYRAKPHTIFLPQHIDADLILVVLSGKAILTVLSPNDRNSYNLERGDTIKLPAGTTS
YLVNQDDEEDLRLVDLVIPVNGPGKFEAFDLAKNKNQYLRGFSKNILEASYNTRYETIEK
VLLEEQEKDRKRRQQGEETDAIVKVS [SEQ ID 8]
PEPTIDE: LVDLVIPVNGPGKFEAFDLAK [SEQ ID 110]
PEA PROTEIN 7: P02855
MATTIKSRFPLLLLLGIIFLASVVSVTYANYDEGSEPRVPAQRERGRQEGEKEEKRHGEW
RPSYEKEEDEEEGQRERGRQEGEKEEKRHGEWGPSYEKQEDEEEKQKYRYQREKEDEEEK
QKYQYQREKKEQKEVQPGRERWEREEDEEQVDEEWRGSQRREDPEERARLRHREERTKRD
RRHQREGEEEERSSESQERRNPFLFKSNKFLTLFENENGHIRLLQRFDKRSDLFENLQNY
RLVEYRAKPHTIFLPQHIDADLILVVLSGKAILTVLSPNDRNSYNLERGDTIKLPAGTTS
YLVNQDDEEDLRLVDLVIPVNGPGKFEAFDLAKNKNQYLRGFSKNILEASYNTRYETIEK
VLLEEQEKDRKRRQQGEETDAIVKVS [SEQ ID 9]
PEPTIDE: KNPQLQDLDIFVNYVEIK [SEQ ID 111]
RICE PROTEIN 1: P14323-1-
MASSVFSRFSIYFCVLLLCHGSMAQLFNPSTNPWHSPRQGSFRECRFDRLQAFEPLRKVRSEAGVTEYFDEKNELFQCTGTFVIRRVIQPQG
LLVPRYTNIPGVVYIIQGRGSMGLTFPGCPATYQQQFQQFSSQGQSQSQKFRDEHQKIHQFRQGDIVALPAGVAHWFYNDGDAPIVAVY
VYDVNNNANQLEPRQKEFLLAGNNNRAQQQQVYGSSIEQHSGQNIFSGFGVEMLSEALGINAVAAKRLQSQNDQRGEIIHVKNGLQLLK
PTLTQQQEQAQAQDQYQQVQYSERQQTSSRWNGLEENFCTIKVRVNIENPSRADSYNPRAGRITSVNSQKFPILNLIQMSATRVNLYQN
AILSPFWNVNAHSLVYMIQGRSRVQVVSNFGKTVFDGVLRPGQLLIIPQHYAVLKKAEREGCQYIAIKTNANAFVSHLAGKNSVFRALPVD
VVANAYRISREQARSLKNNRGEEHGAFTPRFQQQYYPGLSNESESETSE [SEQ ID 10]
REGION: 138 to 159-SQSQKFRDEHQKIHQFRQGDIV [SEQ ID 34]
PEPTIDE: DEHQKIHQFR [SEQ ID 112]
PEPTIDE: FRDEHQK [SEQ ID 113]
REGION: 336 to 358-VNSQKFPILNLIQMSATRVNLYQ [SEQ ID 35]
PEPTIDE: FPILNLIQMSATR [SEQ ID 114]
REGION: 423 to 462-YIAIKTNANAFVSHLAGKNSVFRALPVDVVANAYRISREQ [SEQ ID 36]
PEPTIDE: TNANAFVSHLAGK [SEQ ID 115]
PEPTIDE: ALPVDVVANAYR [SEQ ID 116]
REGION: 175 to 205-APIVAVYVYDVNNNANQLEPRQKEFLLAGNN [SEQ ID 37]
PEPTIDE: YVYDVNNNANQLEPRQKEFL [SEQ ID 117]
PEPTIDE: VYVYDVNNNANQLEPRQKEFL [SEQ ID 118]
REGION: 318 to 334-ENPSRADSYNPRAGRIT [SEQ ID 38]
PEPTIDE: ADSYN PR [SEQ ID 119]
REGION: 265 to 296-GLQLLKPTLTQQQEQAQAQDQYQQVQYSERQQ [SEQ ID 39]
PEPTIDE: KPTLTQQQEQAQAQDQ [SEQ ID 120]
PEPTIDE: QAQAQDQYQQVQY [SEQ ID 121]
PEPTIDE: QAQDQYQQVQY [SEQ ID 122]
REGION: 45 to 62-CRFDRLQAFEPLRKVRSE [SEQ ID 40]
PEPTIDE: LQAFEPLR [SEQ ID 123]
REGION: 361 to 408-ILSPFWNVNAHSLVYMIQGRSRVQVVSNFGKTVFDGVLRPGQLLIIPQ [SEQ ID 41]
PEPTIDE: SRVQVVSNFGK [SEQ ID 124]
PEPTIDE: WNVNAHSLVY [SEQ ID 125]
PEPTIDE: NVNAHSLVY [SEQ ID 126]
PEPTIDE: IQGRSRVQVVSNFGK [SEQ ID 127]
PEPTIDE: GKTVFDGVLRPGQL [SEQ ID 128]
PEPTIDE: FGKTVFDGVLRPGQL [SEQ ID 129]
REGION: 476 to 499-AFTPRFQQQYYPGLSNESESETSE [SEQ ID 42]
PEPTIDE: FQQQYYPGLSNESESETSE [SEQ ID 130]
PEPTIDE: QQYYPGLSN [SEQ ID 131]
PEPTIDE: QQQYYPGLSN [SEQ ID 132]
RICE PROTEIN 2: P07728-2-
MASINRPIVFFTVCLFLLCNGSLAQQLLGQSTSQWQSSRRGSPRECRFDRLQAFEPIRSVRSQAGTTEFFDVSNEQFQCTGVSVVRRVIEPR
GLLLPHYTNGASLVYIIQGRGITGPTFPGCPESYQQQFQQSGQAQLTESQSQSQKFKDEHQKIHRFRQGDVIALPAGVAHWCYNDGEVPV
VAIYVTDLNNGANQLDPRQRDFLLAGNKRNPQAYRREVEERSQNIFSGFSTELLSEALGVSSQVARQLQCQNDQRGEIVRVEHGLSLLQPY
ASLQEQEQGQVQSRERYQEGQYQQSQYGSGCSNGLDETFCTLRVRQNIDNPNRADTYNPRAGRVTNLNTQNFPILSLVQMSAVKVNLY
QNALLSPFWNINAHSVVYITQGRARVQVVNNNGKTVFNGELRRGQLLIIPQHYAVVKKAQREGCAYIAFKTNPNSMVSHIAGKSSIFRAL
PNDVLANAYRISREEAQRLKHNRGDEFGAFTPIQYKSYQDVYNAAESS [SEQ ID 11]
REGION: 332 to 362-PRAGRVTNLNTQNFPILSLVQMSAVKVNLYQ [SEQ ID 43]
PEPTIDE: VTNLNTQNFPILSLVQMSAVK [SEQ ID 133]
REGION: 375 to 406-HSVVYITQGRARVQVVNNNGKTVFNGELRRGQ [SEQ ID 44]
PEPTIDE: ITQGRARVQVVNNNGKTVF [SEQ ID 134]
PEPTIDE: ITQGRARVQVVNNNGKTVFNGE [SEQ ID 135]
PEPTIDE: ITQGRARVQVVNNNGKTVFNG [SEQ ID 136]
PEPTIDE: RVQVVNNNGKTVF [SEQ ID 137]
REGION: 440 to 471-HIAGKSSIFRALPNDVLANAYRISREEAQRLK [SEQ ID 45]
PEPTIDE: RALPNDVLANAYRISREE [SEQ ID 138]
PEPTIDE: SIFRALPNDVLANAYR [SEQ ID 139]
PEPTIDE: SIFRALPNDVLANAY [SEQ ID 140]
PEPTIDE: SIFRALPNDVLAN [SEQ ID 141]
PEPTIDE: SSIFRALPNDVLANAYR [SEQ ID 142]
PEPTIDE: SIFRALPNDVLANAYRISREE [SEQ ID 143]
PEPTIDE: SIFRALPND [SEQ ID 144]
REGION: 180 to 208-VPVVAIYVTDLNNGANQLDPRQRDFLLAG [SEQ ID 46]
PEPTIDE: IYVTDLNNGANQLDPRQRD [SEQ ID 145]
RICE PROTEIN 3: P07730-2-
MASINRPIVFFTVCLFLLCDGSLAQQLLGQSTSQWQSSRRGSPRGCRFDRLQAFEPIRSVRSQAGTTEFFDVSNELFQCTGVSVVRRVIEPR
GLLLPHYTNGASLVYIIQGRGITGPTFPGCPETYQQQFQQSGQAQLTESQSQSHKFKDEHQKIHRFRQGDVIALPAGVAHWCYNDGEVPV
VAIYVTDINNGANQLDPRQRDFLLAGNKRNPQAYRREVEEWSQNIFSGFSTELLSEAFGISNQVARQLQCQNDQRGEIVRVERGLSLLQPY
ASLQEQEQGQMQSREHYQEGGYQQSQYGSGCPNGLDETFCTMRVRQNIDNPNRADTYNPRAGRVTNLNSQNFPILNLVQMSAVKVN
LYQNALLSPFWNINAHSIVYITQGRAQVQVVNNNGKTVFNGELRRGQLLIVPQHYVVVKKAQREGCAYIAFKTNPNSMVSHIAGKSSIFR
ALPTDVLANAYRISREEAQRLKHNRGDEFGAFTPLQYKSYQDVYNVAESS [SEQ ID 12]
REGION: 311 to 362-TFCTMRVRQNIDNPNRADTYNPRAGRVTNLNSQNFPILNLVQMSAVKVNLYQ [SEQ ID 47]
PEPTIDE: VTNLNSQNFPILNLVQMSAVK [SEQ ID 146]
PEPTIDE: QNIDNPNR [SEQ ID 147]
PEPTIDE: ADTYNPR [SEQ ID 148]
PEPTIDE: NIDNPNRADTYNPRAGRVTNL [SEQ ID 149]
PEPTIDE: RVRQNIDNPNRADTYNPRAGRVTNL [SEQ ID 150]
REGION: 427 to 499-
YIAFKTNPNSMVSHIAGKSSIFRALPTDVLANAYRISREEAQRLKHNRGDEFGAFTPLQYKSYQDVYNVAESS [SEQ ID 48]
PEPTIDE: TNPNSMVSHIAGKSSIFR [SEQ ID 151]
PEPTIDE: HNRGDEFGAFTPLQYK [SEQ ID 152]
PEPTIDE: SYQDVYNVAESS [SEQ ID 153]
PEPTIDE: ISREEAQR [SEQ ID 154]
PEPTIDE: SIFRALPTDVLANAYRISREE [SEQ ID 155]
PEPTIDE: YRISREEAQRLKHNRGDEF [SEQ ID 156]
PEPTIDE: YRISREEAQRLKHNRGDE [SEQ ID 157]
REGION: 143 to 178-SQSHKFKDEHQKIHRFRQGDVIALPAGVAHWCYNDG [SEQ ID 49]
PEPTIDE: FKDEHQKIHR [SEQ ID 158]
PEPTIDE: QGDVIALPAGVAHW [SEQ ID 159]
REGION: 381 to 409-TQGRAQVQVVNNNGKTVFNGELRRGQLLI [SEQ ID 50]
PEPTIDE: TVFNGELRR [SEQ ID 160]
PEPTIDE: TVFNGELR [SEQ ID 161]
PEPTIDE: QVQVVNNNGKTVF [SEQ ID 162]
REGION: 101 to 124-NGASLVYIIQGRGITGPTFPGCPE [SEQ ID 51]
PEPTIDE: YIIQGRGITGPTF [SEQ ID 163]
PEPTIDE: VYIIQGRGITGPTF [SEQ ID 164]
REGION: 46 to 66-CRFDRLQAFEPIRSVRSQAGT [SEQ ID 52]
PEPTIDE: LQAFEPIRSVR [SEQ ID 165]
REGION: 253 to 292-QNDQRGEIVRVERGLSLLQPYASLQEQEQGQMQSREHYQE [SEQ ID 53]
PEPTIDE: GLSLLQPYASLQEQEQGQMQSR [SEQ ID 166]
PEPTIDE: GEIVRVER [SEQ ID 167]
PEPTIDE: RGLSLLQPYASLQ [SEQ ID 168]
PEPTIDE: RGLSLLQPYASLQEQ [SEQ ID 169]
PEPTIDE: RGLSLLQPYASLQEQE [SEQ ID 170]
PEPTIDE: RGLSLLQPYASLQE [SEQ ID 171]
REGION: 199 to 233-PRQRDFLLAGNKRNPQAYRREVEEWSQNIFSGFST [SEQ ID 54]
PEPTIDE: RNPQAYR [SEQ ID 172]
PEPTIDE: FLLAGNKRNPQAY [SEQ ID 173]
PEPTIDE: EVEEWSQNIF [SEQ ID 174]
PEPTIDE: LAGNKRNPQAYR [SEQ ID 175]
PEPTIDE: FLLAGNKRNPQA [SEQ ID 176]
RICE PROTEIN 4: Q0D7S0-3-
MASNKVVFSVLLLAVVSVLAATATMAEYHHQDQVVYTPGPLCQPGMGYPMYPLPRCRALVKRQCVGRGTAAAAEQVRRDCCRQLAAV
DDSWCRCEAISHMLGGIYRELGAPDVGHPMSEVFRGCRRGDLERAAASLPAFCNVDIPNGGGGVCYWLARSGY [SEQ ID 13+
REGION: 102 to 124-GGIYRELGAPDVGHPMSEVFRGC [SEQ ID 55]
PEPTIDE: ELGAPDVGHPMSE [SEQ ID 177]
RICE PROTEIN 5: P14614-4-
MATIAFSRLSIYFCVLLLCHGSMAQLFGPNVNPWHNPRQGGFRECRFDRLQAFEPLRRVRSEAGVTEYFDEKNEQFQCTGTFVIRRVIEPQ
GLLVPRYSNTPGMVYIIQGRGSMGLTFPGCPATYQQQFQQFLPEGQSQSQKFRDEHQKIHQFRQGDIVALPAGVAHWFYNEGDAPVVA
LYVFDLNNNANQLEPRQKEFLLAGNNNREQQMYGRSIEQHSGQNIFSGFNNELLSEALGVNALVAKRLQGQNDQRGEIIRVKNGLKLLRP
AFAQQQEQAQQQEQAQAQYQVQYSEEQQPSTRCNGLDENFCTIKARLNIENPSHADTYNPRAGRITRLNSQKFPILNLVQLSATRVNLY
QNAILSPFWNVNAHSLVYIVQGHARVQVVSNLGKTVFNGVLRPGQLLIIPQHYVVLKKAEHEGCQYISFKTNANSMVSHLAGKNSIFRAM
PVDVIANAYRISREQARSLKNNRGEELGAFTPRYQQQTYPGFSNESENEALE [SEQ ID 14]
REGION: 372 to 397-HSLVYIVQGHARVQVVSNLGKTVFNG [SEQ ID 56]
PEPTIDE: IVQGHARVQVVSNLGK [SEQ ID 178]
PEPTIDE: IVQGHARVQVVSNL [SEQ ID 179]
PEPTIDE: IVQGHARVQVVSN [SEQ ID 180]
REGION: 464 to 486-ARSLKNNRGEELGAFTPRYQQQT [SEQ ID 57]
PEPTIDE: NNRGEELGAFTPR [SEQ ID 181]
PEPTIDE: GEELGAFTPR [SEQ ID 182]
REGION: 337 to 359-LNSQKFPILNLVQLSATRVNLYQ [SEQ ID 58]
PEPTIDE: FPILNLVQLSATR [SEQ ID 183]
REGION: 210 to 293-
QMYGRSIEQHSGQNIFSGFNNELLSEALGVNALVAKRLQGQNDQRGEIIRVKNGLKLLRPAFAQQQEQAQQQEQAQAQYQVQYS [SEQ ID
59]
PEPTIDE: SIEQHSGQNIFSGFNNELLSEALGVNALVAK [SEQ ID 184]
PEPTIDE: LQGQNDQR [SEQ ID 185]
PEPTIDE: SGFNNELLSEALGVNALVAK [SEQ ID 186]
PEPTIDE: PAFAQQQEQAQQQEQAQAQY [SEQ ID 187]
PEPTIDE: VAKRLQGQNDQRGEI [SEQ ID 188]
PEPTIDE: ALVAKRLQGQNDQRGEI [SEQ ID 189]
PEPTIDE: LQGQNDQRGEIIR [SEQ ID 190]
REGION: 24 to 47-AQLFGPNVNPWHNPRQGGFRECRF [SEQ ID 60]
PEPTIDE: PNVNPWHNPRQGGF [SEQ ID 191]
REGION: 164 to 186-GVAHWFYNEGDAPVVALYVFDLN [SEQ ID 61]
PEPTIDE: FYNEGDAPVVALY [SEQ ID 192]
PEPTIDE: FYNEGDAPVV [SEQ ID 193]
PEPTIDE: FYNEGDAPVVAL [SEQ ID 194]
PEPTIDE: FYNEGDAPVVA [SEQ ID 195]
REGION: 424 to 463-YISFKTNANSMVSHLAGKNSIFRAMPVDVIANAYRISREQ [SEQ ID 62]
PEPTIDE: TNANSMVSHLAGK [SEQ ID 196]
PEPTIDE: AMPVDVIANAYR [SEQ ID 197]
RICE PROTEIN 6: QODEV5-5-
MSALTTSQLATSATGFGIADRSAPSSLLRHGFQGLKPRSPAGGDATSLSVTTSARATPKQQRSVQRGSRRFPSVVVYATGAGMNVVFVGA
EMAPWSKTGGLGDVLGGLPPAMAANGHRVMVISPRYDQYKDAWDTSVVAEIKVADRYERVRFFHCYKRGVDRVFIDHPSFLEKVWGK
TGEKIYGPDTGVDYKDNQMRFSLLCQAALEAPRILNLNNNPYFKGTYGEDVVFVCNDWHTGPLASYLKNNYQPNGIYRNAKVAFCIHNIS
YQGRFAFEDYPELNLSERFRSSFDFIDGYDTPVEGRKINWMKAGILEADRVLTVSPYYAEELISGIARGCELDNIMRLTGITGIVNGMDVSE
WDPSKDKYITAKYDATTAIEAKALNKEALQAEAGLPVDRKIPLIAFIGRLEEQKGPDVMAAAIPELMQEDVQIVLLGTGKKKFEKLLKSMEE
KYPGKVRAVVKFNAPLAHLIMAGADVLAVPSRFEPCGLIQLQGMRYGTPCACASTGGLVDTVIEGKTGFHMGRLSVDCKVVEPSDVKKV
AATLKRAIKVVGTPAYEEMVRNCMNQDLSWKGPAKNWENVLLGLGVAGSAPGIEGDEIAPLAKENVAAP [SEQ ID 15]
REGION: 571 to 608-KGPAKNWENVLLGLGVAGSAPGIEGDEIAPLAKENVAA [SEQ ID 63]
PEPTIDE: NWENVLLGLGVAGSAPGIEGDEIAPLAK [SEQ ID 198]
PEPTIDE: NVLLGLGVAGSAPGIEGDE [SEQ ID 199]
PEPTIDE: NWENVLLGLGVAGSAPGIEGDEIAPLAK [SEQ ID 200]
REGION: 458 to 488-RAVVKFNAPLAHLIMAGADVLAVPSRFEPCG [SEQ ID 64]
PEPTIDE: FNAPLAHLIMAGADVLAVPSR [SEQ ID 201]
PEPTIDE: FNAPLAHLIM [SEQ ID 202]
PEPTIDE: FNAPLAHLIMAGADVLAVPSR [SEQ ID 203]
REGION: 545 to 566-KRAIKVVGTPAYEEMVRNCMNQ [SEQ ID 65]
PEPTIDE: VVGTPAYEEMVR [SEQ ID 204]
REGION: 93 to 147-APWSKTGGLGDVLGGLPPAMAANGHRVMVISPRYDQYKDAWDTSVVAEIKVADRY [SEQ ID 66]
PEPTIDE: TGGLGDVLGGLPPAMAANGHR [SEQ ID 205]
PEPTIDE: YDQYKDAWDTSVVAEIK [SEQ ID 206]
PEPTIDE: DAWDTSVVAEIK [SEQ ID 207]
PEPTIDE: VMVISPR [SEQ ID 208]
REGION: 305 to 413-
INWMKAGILEADRVLTVSPYYAEELISGIARGCELDNIMRLTGITGIVNGMDVSEWDPSKDKYITAKYDATTAIEAKALNKEALQAEAGLPVDR
KIPLIAFIGRLEEQK [SEQ ID 67]
PEPTIDE: LTGITGIVNGMDVSEWDPSKDK [SEQ ID 209]
PEPTIDE: VLTVSPYYAEELISGIAR [SEQ ID 210]
PEPTIDE: EALQAEAGLPVDRK [SEQ ID 211]
PEPTIDE: YDATTAIEAK [SEQ ID 212]
PEPTIDE: IPLIAFIGR [SEQ ID 213]
PEPTIDE: AGILEADR [SEQ ID 214]
PEPTIDE: IPLIAFIGR [SEQ ID 215]
REGION: 158 to 202-RGVDRVFIDHPSFLEKVWGKTGEKIYGPDTGVDYKDNQMRFSLLC [SEQ ID 68]
PEPTIDE: VFIDHPSFLEK [SEQ ID 216]
PEPTIDE: GPDTGVDYKDNQM [SEQ ID 217]
PEPTIDE: IYGPDTGVDYKDNQMR [SEQ ID 218]
PEPTIDE: IYGPDTGVDYK [SEQ ID 219]
REGION: 206 to 226-LEAPRILNLNNNPYFKGTYGE [SEQ ID 69]
PEPTIDE: ILNLNNNPYFK [SEQ ID 220]
PROTEIN P02855
PEPTIDE: KNPQLQDLDI [SEQ ID 356]
PROTEIN P07730
PEPTIDE: GQSTSQWQSSR [SEQ ID 357]
PEPTIDE: QSTSQWQSSR [SEQ ID 358]
PROTEIN P07728
PEPTIDE: QSTSQWQSSR (also in P07730) [SEQ ID 359]
PROTEIN P13918
PEPTIDE: EEEEQGEEEINK [SEQ ID 360]
PROTEIN P14323
PEPTIDE: PSTNPWHSPR [SEQ ID 361]
PEPTIDE: AQAQDQYQQVQYSE [SEQ ID 362]
PEPTIDE: SEAGVTEYFDEKNELFQCTGTFVIRR [SEQ ID 363]
PEPTIDE: QAQAQDQYQQVQYSE [SEQ ID 363]
PEPTIDE: GSMGLTFPGCPAT (also in P14614) [SEQ ID 365]
PEPTIDE: GSMGLTFPGCPATY (also in P14614) [SEQ ID 366]
PROTEIN P14614
PEPTIDE: LGAFTPRY [SEQ ID 367]
PEPTIDE: LGAFTPRYQQ [SEQ ID 368]
PEPTIDE: ALGVNALVAKRLQGQN [SEQ ID 369]
PEPTIDE: LGAFTPRYQ [SEQ ID 370]
PEPTIDE: GSMGLTFPGCPAT (also in P14323) [SEQ ID 371]
PEPTIDE: GSMGLTFPGCPATY (also in P14323) [SEQ ID 372]
PROTEIN P15838
PEPTIDE: SNNPFKFLVPARQS [SEQ ID 373]
PROTEIN Q6K508
PEPTIDE: CAGVFVIRR [SEQ ID 374]
PROTEIN Q6K7K6
PEPTIDE: GSPLQSPRGF [SEQ ID 375]
PEPTIDE: RSSWQQQSY [SEQ ID 376]
PEPTIDE: SFGGSPLQSPR [SEQ ID 377]
PEPTIDE: YLPTKQLQPTW [SEQ ID 378]
PEPTIDE: GKPRSSWQQQ [SEQ ID 379]
PEPTIDE: FGGSPLQSPRG [SEQ ID 380]
PROTEIN Q9M3X6
PEPTIDE: LNLLGFGINAENNE [SEQ ID 381]
ADDITIONAL PEPTIDES [SEQ ID 381-417]
LRGFSK [381]
GALMLPHYN
GALMLPHYNSR
VFDGVLRPG
LQSQND
LQSQNDQRGEI
QSQNDQRGEIIHVK
RGEIIHVK
RLQSQNDQ
RLQSQNDQRG [SEQ ID 390]
RLQSQNDQRGEIIH
MPMP
PMPL
LEPDNR
GIARLAGTSSVIN
RSQNIF
PNSM
GHPM
HPMS
FLPQHTD [SEQ ID 400]
EWQINEK
GPQQYAEWQINEK
PQQYAEWQ
RGPQQYA
HNPR
WHN
WDP
HPSF
PGQLQSFLLSGNQNQQNYLSGF
QLQSFLLSGNQNQQNYLSGFSK [SEQ ID 410]
QSFLLSGNQNQQ
PGQLQSFLLSGN
QSFLLSGNQ
QNQQNYLSGFSK
YLRGFS
PVEMPTLLYPS
RGPQQYAEWQINE [SEQ ID 417]
TVFDGVLRPGQL
LDALEPDNR
RLQSQNDQRGEIIHVK [SEQ ID 420]
VLDLAIPVNRPGQL
HGPVEMPYTLLYPSSK [SEQ ID 422]
GYYGEQQQQPGMTR [SEQ ID 423] PROTEIN: P29835-5-RICE
SEEGYYGEQQQQPGMTR [SEQ ID 424] PROTEIN: P29835-5-RICE
PROTEIN: P29835-5-RICE
MASKVVFFAAALMAAMVAISGAQLSESEMRFRDRQCQREVQDSPLDACRQVLDRQLTGRERFQPMFRRPGALGLRMQCCQQLQDVSRE
CRCAAIRRMVRSYEESMPMPLEQGWSSSSSEYYGGEGSSSEQGYYGEGSSEEGYYGEQQQQPGMTRVRLTRARQYAAQLPSMCRVEPQQC
SIFAAGQY [SEQ ID 353]
PROTEIN: P02857-1-PEA
MAKLLALSLSFCFLLLGGCFALREQPQQNECQLERLDALEPDNRIESEGGLIETWNPNNKQFRCAGVALSRATLQRNALRRPYYSNAPQEIFIQ
QGNGYFGMVFPGCPETFEEPQESEQGEGRRYRDRHQKVNRFREGDIIAVPTGIVFWMYNDQDTPVIAVSLTDIRSSNNQLDQMPRRFYLAG
NHEQEFLQYQHQQGGKQEQENEGNNIFSGFKRDYLEDAFNVNRHIVDRLQGRNEDEEKGAIVKVKGGLSIISPPEKQARHQRGSRQEEDED
EEKQPRHQRGSRQEEEEDEDEERQPRHQRRRGEEEEEDKKERGGSQKGKSRRQGDNGLEETVCTAKLRLNIGPSSSPDIYNPEAGRIKTVTSL
DLPVLRWLKLSAEHGSLHKNAMFVPHYNLNANSIIYALKGRARLQVVNCNGNTVFDGELEAGRALTVPQNYAVAAKSLSDRFSYVAFKTNDR
AGIARLAGTSSVINNLPLDVVAATFNLQRNEARQLKSNNPFKFLVPARESENRASA [SEQ ID 354]
PROTEIN: P09918-14-Pisum sativum
MFSGVTGILNRGHKIKGTVVLMRKNVLDINSLTTVGGVIGQGFDILGSTVDNLTAFLGRSVSLQLISATKPDATGKGKLGKATFLEGIISSLPTLG
AGQSAFKIHFEWDDDMGIPGAFYIKNFMQTEFFLVSLTLDDIPNHGSIYFVCNSWIYNAKHHKIDRIFFANQTYLPSETPAPLVHYREEELNNLR
GDGTGERKEWERIYDYDVYNDLGNPDSGENHARPVLGGSETYPYPRRGRTGRKPTRKDPNSESRSDYVYLPRDEAFGHLKSSDFLTYGLKAVS
QNVVPALESVFFDLNFTPNEFDSFDEVHGLYEGGIKLPTNILSQISPLPVLKEIFRTDGENTLKYPPPKVIQVSRSGWMTDEEFAREMLAGVNPN
VICCLQEFPPRSKLDSQIYGDHTSKISKEHLEPNLEGLTVEEAIQNKKLFLLDHHDSIMPYLRRINSTSTKAYATRTILFLNNNQNLKPLAIELSLPHP
QGDEHGAVSYVYQPALEGVESSIWLLAKAYVIVNDSCYHQLVSHWLNTHAVVEPFVIATNRHLSCLHPIYKLLYPHYRDTMNINSLARLSLVND
GGIIEKTFLWGRYSMEMSSKVYKNWVFTEQALPADLIKRGMAIEDPSSPCGVKLVVEDYPYAVDGLEIWAIIKTWVQDYVSLYYTSDEKLRQD
SELQAWWKELVEVGHGDKKNEPWWPKMQTREDLIEVCSIVIWTASALHAAVNFGQYSYGGLILNRPTLSRRFMPEKGSAEFEELVKSPQKA
YLKTITPKFQTLIDLSVIEILSRHASDELYLGERDNPNWTSDKRALEAFKKFGNKLAEIEKKLTQRNNDEKLRNRHGPVEMPYTLLYPSSKEGLTFR
GIPNSISI [SEQ ID 355]
BACTERIAL PROTEIN 1: POC1U8-5-
MKGKFLKVSSLFVATLTTATLVSSPAANALSSKAMDNHPQQTQSSKQQTPKIQKGGNLKPLEQREHANVILPNNDRHQITDTTNGHYAPV
TYIQVEAPTGTFIASGVVVGKDTLLTNKHVVDATHGDPHALKAFPSAINQDNYPNGGFTAEQITKYSGEGDLAIVKFSPNEQNKHIGEVVK
PATMSNNAETQVNQNITVTGYPGDKPVATMWESKGKITYLKGEAMQYDLSTTGGNSGSPVFNEKNEVIGIHWGGVPNEFNGAVFINEN
VRNFLKQNIEDIHFANDDQPNNPDNPDNPNNPDNPNNPDEPNNPDNPNNPDNPDNGDNNNSDNPDAA [SEQ ID 16]
REGION: 92 to 117-YIQVEAPTGTFIASGVVVGKDTLLTN [SEQ ID 70]
PEPTIDE: APTGTFIASGVVVGKD [SEQ ID 221]
Homologs of Pea protein 1 (SEQ ID 1)
>gi|137584|sp|P08438.1| VCL_VICFA RecName: Full=Vicilin; Flags: Precursor [Vicia faba]
>gi|22057|emb|CAA68559.1| vicilin [Vicia faba var. minor] >gi|383931031|gb|AFH56916.1| vicilin
[Vicia faba]
MAATTLKDSFPLLTLLGIAFLASVCLSSRSDQDNPFVFESNRFQTLFENENGHIRLLQKFDQHSKLLENLQNYRLLEYKSKPHTIFLPQQTDADFIL
VVLSGKAILTVLLPNDRNSFSLERGDTIKLPAGTIGYLVNRDDEEDLRVLDLVIPVNRPGEPQSFLLSGNQNQPSILSGFSKNILEASFNTDYKEIEK
VLLEEHGKEKYHRRGLKDRRQRGQEENVIVKISRKQIEELNKNAKSSSKKSTSSESEPFNLRSREPIYSNKFGKFFEITPKRNPQLQDLNIFVNYVEI
NEGSLLLPHYNSRAIVIVTVNEGKGDFELVGQRNENQQGLREEYDEEKEQGEEEIRKQVQNYKAKLSPGDVLVIPAGYPVAIKASSNLNLVGFG1
NAENNQRYFLAGEEDNVISQIHKPVKELAFPGSAQEVDTLLENQKQSHFANAQPRERERGSQEIKDHLYSI LGSF [SEQ ID 222]
>gi|502105533|ref|XP_004492829.1| PREDICTED: vicilin-like isoform X1 [Cicer arietinum] ChickPea
MAIKARFPLLVLLGIVFLASVCAKSDKENPFFFKSNNCQTLFENENGHVRLLQRFDKRSQLFENLQNYRLMEYNSKPHTLFLPQHNDADFILVVL
RGRAILTVLNPNDRNTFKLERGDTIKLPAGTIAYLANRDDNEDLRVLDLAIPVNRPGQFQSFSLSGNENQQSYFQGFSKKILEASFNSDYEEIERV
LLEEQEQKPEQRRGHKGRQQSQETDVIVKISREQIEELSKNAKSNCKKSVSSESEPFNLRSRSPIYSNRFGNFFEITPEKNPQLKDLDIFVNSVEIK
EGSLLLPHFNSRATVILVVNEGKGEVELVGLRNENEQENKKEDEEEEEDRNVQVQRFQSKLSSGDVVVIPASHPFSINASSDLFLLGFG1NAQN
NQRNFLAGEEDNVISQIQRPVKEVAFPGSAEEVDRLLKNQRQSHFANAQPQQKRKGSQRIRSPF [SEQ ID 223]
>gi|29539109|emb|CAD87730.1| allergen Len c 1.0101 [Lens culinaris] Lenti]
SRSDQENPFIFKSNRFQTIYENENGHIRLLQRFDKRSKIFENLQNYRLLEYKSKPHTIFLPQFTDADFILVVLSGKAILTVLNSNDRNSFNLERGDTI
KLPAGTIAYLANRDDNEDLRVLDLAIPVNRPGQLQSFLLSGTQNQPSFLSGFSKNILEAAFNTEYEEIEKVLLEEQEQKSQHRRSLRDKRQEITNE
DVIVKVSREQIEELSKNAKSSSKKSVSSESEPFNLRSRNPIYSNKFGKFFEITPEKNPQLQDLDIFVNSVEIKEGSLLLPNYNSRAIVIVTVNEGKGDF
ELVGQRNENQQEQREENDEEEGQEEETTKQVQRYRARLSPGDVLVIPAGHPVAINASSDLNLIGFGINAKNNQRNFLAGEEDNVISQIQRPV
KELAFPGSSREVDRLLTNQKQSHFANAQPLQIE [SEQ ID 224]
Homologs of Pea protein 2 (SEQ ID 2)
>gi|29539111|emb|CAD87731.1| allergen Len c 1.0102 [Lens culinaris]
SRSDQENPFIFKSNRFQTIYENENGHIRLLQKFDKRSKIFENLQNYRLLEYKSKPHTLFLPQYTDADFILVVLSGKAVLTVLNSNDRNSFNLERGDT
IKLPAGTIAYLANRDDNEDLRVLDLAIPVNNPGQLESFLLSGTQNQPSFLSGFNKSILEAAFNTDYEEIEKVLLEDQEQEPQHRRSLRDRRQEINK
ENVIVKVSREQIKELSKNAKSSSKKSVSSESEPFNLRSRNPIYSNKFGKFFEITPEKNPQLQDLDIFVNSVEIKEGSLLLPNYNSRAIVIVTVNEGKGY
FELVGQRNENQREENDDEEEQEEETSTQVQRYRAKLSPGDVFVVPAGHPVAINASSDLNLIGFGINAKNNQRNFLAGEEDNVISQIQRPVKEL
AFPGSSREVDRLLTNQKQSHFANAQPLQIE [SEQ ID 225]
>gi|1297072|emb|CAA96514.1| vicilin precursor [Vicia narbonensis]
MAAITMKVSFPLLMLLGISFLASVCVSSRSDQENPFIFKSNKFQTLFENDNGHIRLLQKFDERSKILENLQNYRLLEYKSKPRTIFLPQQTNADFIL
VVLSGKAILTVLKPDDRNSFNLERGDTIKLPAGTIAYLVNKDDNEDLRVLDLAIPVNGPDQLQSFLLSGSENQQSILSGFSKSVLEASFNTGYEEIE
KVLLEEREKETQHRRSLRDKRQHSQDEDVIVKLSRGQIEELSRNAKSSSKKSVSSESEPFNLRSRNPIYSNKFGKFFEITPEKNPQLQDLDVLVNSV
EIKEGSLLLPHYNSRAIVIVTVNDGKGDFEIVGQRNENRQGQRKEDDEEEEQGDENTNTQVQNYKAKLSRGDVFVIPAGHPVSIKASSNLDLLG
FGINAKNNQRNFLAGEEDNVISQIDRPVKELAFPGSAQEVDRLLENQKQSHFANAQPQQRERGSHETRDHLSSILDAF [SEQ ID 226]
>gi|28629838|gb|AA045103.1| beta-conglycinin alpha' subunit [Glycine max]
QYGHVRVLQRFNKRSQQLQNLRDYRILEFNSKPNTLLLPHHADADYLIVILNGTAILTLVNNDDRDSYNLQSGDALRVPAGTTYYVVNPDNDE
NLRMITLAIPVNKPGRFESFFLSSTQAQQSYLQGFSKNILEASYDTKFEEINKVLFGREEGQQQGEERLQESVIVEISKKQIRELSKHAKSSSRKTIS
SEDKPFNLRSRDPIYSNKLGKLFEITPEKNPQLRDLDVFLSVVDMNEGALFLPHFNSKAIVVLVINEGEANIELVGIKEQQQRQQQEEQPLEVRK
YRAELSEQDIFVIPAGYPVVVNATSDLNFFAFGINAENNQRNFLAGSKDNVISQIPSQVQELAFPGSAKDIENLIKSQSESYFVDAQPQQKEEGN
KGRKGPLSSILRAFY [SEQ ID 227]
Homologs of Pea protein 3 (SEQ ID 3)
>gi|483449|emb|CAA83677.1| legumin A [Vicia sativa]
MAKLLALSLSFCFLLFSSCFALREQSQQNECQLERINALEPDNRIESEGGLIETWNPNNRQFRCARVALSRATLQRNALRRPYYSNAPQEIYIQQ
GNGYFGMVFPGCPETHEEPQQSEQGEGRRYRDSHQKVNRFREGDIIAVPTGIAFWMYNDQDTPVIAISLTDTGSSNNQLDQMPRRFYLAG
NQEQEFLRYQHQQGGKQEQDNDGNNIFSGFKRDFLEDAFNVNRHIVDRLQGRNEDEEKGAIVKVKGGLSIIAPPERQARHERGSRQEEDED
EKEERQPSHHKSRRDEDEDDKEKRHSQKGQSRRQGDNGLEETVCTAKLRANIGSSPSPDIYNPQAGRIKTVTSLDLPVLRWLKLSAEHGSLHK
NAMFVPHYNLNANSVIYALKGRARLQVVNCNGNTVFDGELEAGRALTVPQNYAVAAKSLSERFTYVAFKTDDRASIARLAGTSSVIDDLPLDV
VAATFNMQRNEARQLKSNNPFKFLVPPRQSEMRASA [SEQ ID 228]
>gi|657379551|gb|KEH23931.1| legumin A2 [Medicago truncatula]
MAKLLALSLSLCFLLFSGCFAIREHQPHQKQQPQQNECQLEQLNALEPDNRIESEGGIIETWNPNNRQFRCAGVALSRCTLQRNSLRRPFYSNA
PQEIFIQQGSGYFGMVFPGCPETFEEPQESEQRESRRIRESEQGESRRIRESEQGEGRRFRDSHQKVNRFREGDLIAVPTGTVFWMYNDQDTP
VIAVSLIDTGSFQNQLDEMPRRFYLAGNQEQEFLQYQQQQVRGRGEQRRGREQQENEGGNIFSGFKRDFLEDALNVNRHIVDRLQGRNEDE
EKGAIVKVRGGLSFVTPPERQSRHQGGSIIEEDEDEEDEWRRPHHQKSRRGEEEERPCRRGQKCERSNGLEETICTARLRQNIGSSSSPDIYNPE
AGRIKTVTSFDLPALRWLRLSAEHGTLHRNAMFVPHYNLNANSAIYALRGRARLQVVNCNGNTVFDGELEAGRVLIVPQNFAVAAKSMSDRF
QYVSFKTNDNAAIARLAGTQSTLSGVPMDVLAATYNMDRNEARQLKNNNLYKFLVPPRESERRAAA [SEQ ID 229]
>gi|206712292|emb|CAR78996.1| legumin storage protein 5 [Lotus japonicus]
MAYKLFALSLSFCFLLFGGCFAIRQQSQQQNECQLERLNALKPDNRIESEAGYIETWNPTNNQFRCAGVALSRCTLRRNGLKRPSYSNAPQEIFI
QQGSGIFGMIFPGCPETVEEPFESDQQGRRDRHQKVNRFREGDVIAVPPGVVFWMYNEEETPVIAVSLIDTGSYLNQLDQMPRRFYLSGNQ
EQEFLQYQRQEVRGREEENQGGNIFSGFGGEFLEDALNIDRNIVHKLQGRDEEQDKGAIVRVKGGLSVITPPERQSHRRGSEEEEDEEEDRPS
RHQSRGGSRRNGLEETICTVRLRMNIGKSSSPDIFNPQAGRIKTATGFDFPALRFLKLSAEHGSLNRNAMVVPHYNLNANSIIYALRGRAWIQV
VNCKGNRIFDGELEEGQVLIVPQNFVVAARSMSDKFNYVAFKTNDMPTMAKLAGATSEIQAMPLEVIQNAFNLEREQAKQVKFNNRFNFLV
PPREQSQRRASA [SEQ ID 230]
Homologs of Pea protein (SEQ ID 4)
>gi|164512526|emb|CAP06312.1| cvc [Pisum abyssinicum]
MATTVESRFPLLLFPGIIFLASVCVTYANYDEGSETRVPGQRERGRQEGEKEEKRHGEWRPSYEKEEDEEEKQKYRYQREKEDEEEKQKYRYQR
EKKEEKEVQPGRERWEREEDEEQVDEEWRGSQRRQDPEERARLRHREERTKRDRRHKREGEEEERSSESQEQRNPFLFKSNKFLTLFENENG
HIRRLQRFDKRSDLFENLQNYRLVEYRAKPHTIFLPQHIDADLILVVLNGKAILTVLSPNDRNSYNLERGDTIKIPAGTTSYLVNQDDEEDLRVVD
FVIPVNRPGKFEAFGLSENKNQYLRGFSKNILEASLNTKYETIEKVLLEEQEKKPQQLRDRKRRQQGGERDAIIKVSREQIEELRKLAKSSSKKSLPS
EFEPFNLRSHKPEYSNKFGKLFEITPEKKYPQLQDLDILVSCVEINKGALMLPHYNSRAIVVLLVNEGKGNLELLGLKNEQQEREDRKERNNEVQ
RYEARLSPGDVVIIPAGHPVAISASSNLNLLGFGTNAENNQRNFLSGSDDN [SEQ ID 231]
>gi|164512538|emb|CAP06318.1| cvc [Lathyrus annuus]
MATTIKSRFPLLLLLGIIFLASVCVTWANYDEGSEPRVPGQRERGRQEGEKEEKRHGEWRPSYEEEYDEGLEPKVPGKRERGRQEGEKEEKRHE
EWRPSYEKEEDEEEKQKYNYQREKKEHKEVQPGRERWERKQDEKQVEEDEEPGEEQWRGSKRHEDPEERARLRHREEKTKSYVEDNEETSS
KEGRNPFLFKSNKFLTLFENENGHIRRLQRFDERSDIFENLQNYRLVEYRAKPHTMFLPQHIDADLILVVLNGKAILTVLSPNDRNSYNLERGDT
VKLPAGTTSYLVNQDDEEDLRVVDLAIPVNRPGKFEAFGLSANKNQYLRGFSKNILEASLNTKYETIEKVLLEERRDQKGRQQGQETNAIVKVSR
EQIEELRKLAKSSSKKSLLSESEPLNLRSQNPKYSNKFGKFFEITPQKKYPQLQDLDVSISCVEINKGALLLPHYNSRSIGILLVNEGKGNLELVGFKN
EQQRQRENEETNKKLQRYEARLSSGDVVVIPEGHPVAISASSNLNLLGFGINAANNQRNFLTGSDDN [SEQ ID 232]
>gi|164512558|emb|CAP06328.1| cvc [Vicia villosa]
MATTIKSRFPVLLLLGIIFLTSVCVTYANYDEGREPSVPGQRERGRQEGEKEEKRHGEWRPSEEDEEEKYKYEEGRVPGQRERGRQEGEKEEKR
HGKWRPSEEEDEEEKYRYEEGSEPRGPGQRETGRQEGEKEKQRPEREPSYEKEEDEEEKQKYQYHREKKEQREVRPGRERFERHEDEEQWRG
IQRHEDPEERARERYRAEIAKRQVEEEREERDIPHEREQRNPFLFKSNKFQTLFQNENGYIRRLQRFDKRSDLFENLQNYRLVEYRAKPHTIFLPQ
HIDADLIIVVLSGRAILTVLSPDDRNSYNLERGDTIKLPAGTTSYLVNQDDEEDLRVVDLAIPVNRPGKVESFLLSGNKNQYLRGFSKNILEASFNT
NYETIERVLLEEQDKESQQSIGQKRRSQRQETNALVKVSREQLEDLKRLAKSSSQEGLSSQFEPINLRSQNPKYSNKFGKVFEITPEKKYPQLQDL
DLFVSSVDIKEGALMLPHYNSRAIVVLLVNEGRGNLELVGLKNEQQEQREKEDEQQERNNQVQRYEARLSPGDVVIIPAGHPVAVRASSDLNL
LAFGINAENNQRNFLAGSDDN [SEQ ID 233]
Homologs of Pea protein 5 (SEQ ID 5)
>gi|357507721|ref|XP_003624149.1| Provicilin [Medicago truncatula] >gi|87162569|gb|ABD28364.1| Cupin,
RmlC-type [Medicago truncatula] >gi|355499164|gb|AES80367.1| vicilin 47 kDa protein [Medicago truncatula]
MAIKAPFQLLMLLGIFFLASVCVSSRDDRHDQENPFFFNANHFQTLFENENGHIRLLQRFDKRSKIFENLQNYRLLEYHSKPHTLFLPQHNDAD
FILAVLSGKAILTVLNPDNRNSFNLERGDTIKLPAGSIAYLANRDDNEDLRVLDLAIPVNRPGKFQSFSLSGSQNQQSFFSGFSKNILEAAFNANY
EEIERVLIEEHEQEPQHRRGLRKDRRQQSQDSNVIVKVSREQIEELSRHAKSSSRRSGSSESAPFNLRSREPIYSNEFGNFFEITPEKNPQLKDLDIL
VNYAEIREGSLLLPHFNSRATVIVVVDEGKGEFELVGQRNENQQEQREEDEQQEEERSQQVQRYRARLSPGDVYVIPAGHPTVVSASSDLSLL
GFGINAENNERNFLAGEEDNVISQIERPVKEVAFPGSAQDVESLLKNQRQSYFANAQPQQREREEGRSQRQRELISSILGVF [SEQ ID 234]
>gi|164512560|emb|CAP06329.1| convicilin [Vicia peregrina]
MATTFKSRFSLLLLLGIIFLAFVCVTCANYDEGSEPRVPGQRERGRQEGEKEEQSRERHPQREPSREKEEDEEEKQKYDEGTEPRVPGQRERGR
QEGEKEEQRRERHPGQREPSQEEDEEREESDRRQEGSSKSEEQRNPFLFKSNKFLTLFQNGNGHIRLLQRFDKRSDLFENLQNYRLLEYRAKPH
TIFLPQHIDADLILVVLSGRAILTVLSPDDRNSYNLERGDTIKLPAGTTSYPLNQDDEEDLRVVDLAISVNRPGKVESFNLSGNKNQYLRGFSENIL
EASFNTKYETIEKVLLEEQDKESQQPRGQRLQRQETNALVKVSREQVEELKRLARTSSKKGVSSEFEPFNLRSHGPKYSNKFGKFFEITPEKKYPQ
LQDLDISVSSVEINEGALFLPHYNSRAIVVVLVDEGKGNLELVGFKNEQQEQREKEDEQEERNKQVQRYEAKLSPGDVVIIPAGHPVAVSASSN
LNLLGFGINAENNQRNFLTGSDDN [SEQ ID 235]
>gi|164512562|emb|CAP06330.1| convicilin [Vicia lutea]
MATTIKLRFPLLLLLGVILLASVCVTCANYDEGSEPRVPGRPEGEKEEKHRGKLRPSYEKEEDEGEKQRYHYEKKEQKEAQPRREKKEQKEEEKQ
VEEESRESQRYEDPGERARERYRAEIIKRQVEKEREERDRRHQREGEEEEGSSKSRNPFLFKSNNFLTLFENENGHIRLLQRFDKRSDLFENLQNY
RLVEYRAKPHTIFLPQHIDADLILVVLSGKAILTVLSPNNRNSYNLKRGDTIKLPAGTTSYLLNSDDEEDLRMVDLAISVNRPGKVESFNLSGNKN
QYLRGFSKNILEASFNTKYETIEKVLLEEQDKESQQSIGQKRISQRQETNALVKVSREQIEEPKRLARSSSRKGVSSEFEPINLRSQRPKYSNKFGKF
YEISPEKKYPQLQDLDVSVSSVEINEGALLLPHYNSRAIVTVLVNEGKGNLELIGFQNEQQGQREKEDEQQHERNKQVQRYDARLSSGDVVIIP
AGHPVAVSASSNLDLLGFGINAENSQRNFLTGSDDN [SEQ ID 236]
Homologs of Rice protein 1 (SEQ ID 6)
>gi|573919041|ref|XP_006647142.1| PREDICTED: glutelin type-B 4-like [Oryza brachyantha]
MATTTFSRFSIYFCVLLLCHGSMAQLFSPTLNPWHSSRRGGSRDCRFDRLQAFEPLRRVRSEAGVTEYFDERNEQFQCTGTFVIRRVIEPQGLL
VPRYTNTPGVVYIMQGRGSMGLTFPGCPATYQQQFQQFLPEGQSQSQKFRDEHQKIHQFRQGDIVALPAGVAHWFYNEGDTPVVALYVFD
INNSANQLEPRQKDFLLAGNNNREQQVYGRSIEKHSGQNIFSGFNHELLSEALGISTLAAKRLQGQNDHRGEIIRVRNGLQLLKPTFTQQQEQ
AQSQYQVQYSEKQQESTRCNGLDENFCTINARLNIENPSRADTYNPRAGRITHLNNQKFPILNLVQMSATRVNLYQNAILSPYWNVNAHSLV
YMVQGHARVQVVSNLGKTVFNSVLRPGQLLIIPQHYVVLKKAEREGCQYIAFKTNANSIVSQLAGKNSILRAMPVDVVANAYRISREQARDLK
NNRGEELGAFTPKFEQQSYPGLSNESESEASE [SEQ ID 237]
>gi|2764800|emb|CAA54153.1| 125 globulin [Avena sativa]
MATTSFPSVLFYSCIFLLYNGSMAQLFGQSFTPWQSSRQGGLKGCKFDRLQAFEPLRQVRSQAGVTEYFDEQNEQFRCTGVFVIRRVIEPQGL
LLPQYHNAPGLVYILQGRGYTGLTFPGCPATFQQQFQPFDQAQDQSQSHLKDEHQRVHRFKQGDVIALPAGIVHWGYNDGDAPVVAIYVF
DVNNNANQLEPRQKEFLLAGNNKEDQQFGQNIFSGFNIQLLSEALGISQQAAQRIQSQKEQRGEIIRVTQALQFLKPTMSQQELVEHQAYQP
IQSQEGQSTQYQVGQSTQYQEGQSTQYQAGQSQDRSFNGLEENFCSLEARQNIGNPKRADTHNPRAGRITRLHGQNFPILNLVQMSATRV
NLYQNAILSPFWNINAHSVVYMIQGHAQVQVVNNNGQTVFNDRLRQGQLLIVPQHYVVLKKAEREGCQYISFKTNPNSMVSHIAGKSSILRA
LPVDVLANAYRISRQEARNLKNNRGQESGVFTPKFTQTSFQPYPEGEDESSLTNKASE [SEQ ID 238]
>gi|357130026|ref|XP_003566659.1| PREDICTED: 12S seed storage globulin 1-like [Brachypodium distachyon]
>gi|357130028|ref|XP_003566660.1| PREDICTED: 12S seed storage globulin 1-like [Brachypodium distachyon]
MAHTSFSSVLSYFCIFLLFHGSMAQVPGQGSTWQSPRQGGSRECSFDRLQTIEPLTQVRSQAGLTEYFDEQNEQFRCAGVSVIRRVIEPRGLLL
PRYHNTPGLVYILEGSGFVGLAFPGCPETFLEQFQQSRQTQSTLGQSQCQSQSQKLGDVHQRVHQFTQGDVVALPAGVAHWFYNGGDAPV
VAVYVFDVNNNANQLEPRQKEFLLAGNYNGVLQSGRNILNGLNAQLLSQAFGINEQTSRIIQNQNDGRGEIVRVEYGLQFLTPVVTQQQQK
QPFLPIEPQEGQSSRNGLEENFCSLEPRQNIEDPNRADTYNPRAGSIARLNGQNFPILNLVQMSATRVNLQKNAIVSPFWNINAHSVVYVIQG
QASVQVVNNQGRNVFNGLLRRGQLLIIPQNYVVLKKAESEGYQYIAFKTNANSMVSHIAGKNSILRALPVDVIANAYRISRQEAQNLKNNRGE
EIGVLTPNFPQSSCQSYPIGDVDSSSTPKAQE [SEQ ID 239]
Homologs of Rice protein 2 (SEQ ID 7)
>gi|222622792|gb|EEE56924.1| hypothetical protein OsJ_06602 [Oryza sativa Japonica Group]
MAQFSFGGSPLQSPRGFRGDQDSRHQCRFEHLTALEATHQQRSEAGFTEYYNIEARNEFRCAGVSVRRLVVESKGLVLPMYANAHKLVYIVQ
GRGVFGMALPGCPETFQSVRSPFEQEVATAGEAQSSIQKMRDEHQQLHQFHQGDVIAVPAGVAHWLYNNGDSPVVAFTVIDTSNNANQL
DPKRREFFLAGKPRSSWQQQSYSYQTEQLSRNQNIFAGFSPDLLSEALSVSKQTVLRLQGLSDPRGAIIRVENGLQALQPSLQVEPVKEEQTQA
YLPTKQLQPTWLRSGGACGQQNVLDEIMCAFKLRKNIDNPQSSDIFNPHGGRITRANSQNFPILNIIQMSATRIVLQNNALLTPHWTVNAHT
VMYVTAGQGHIQVVDHRGRSVFDGELHQQQILLIPQNFAVVVKARREGFAWVSFKTNHNAVDSQIAGKASILRALPVDVVANAYRLSREDS
RHVKFNRGDEMAVFAPRRGPQQYAEWQINEK [SEQ ID 240]
>gi|218190679|gb|EEC73106.1| hypothetical protein Os1_07091 [Oryza sativa Indica Group]
MAQFSFGGSPLQSPRGFRGDQDSRHQCRFEHLTALEATHQQRSEAGFTEYYNIEARNEFRCAGVSVRRLVVESKGLVLPMYANAHKLVYIVQ
GRGVFGMALPGCPETFQSVRSPFEQEVATAGEAQSSMQKMRDEHQQLHQFHQGDVIAVPAGVAHWLYNNGDSPVVAFTVIDTSNNANQ
LDPKRREFFLAGKPRSSWQQQSYSYQTEQLSRNQNIFAGFNPDLLSEALSVSKQTVLRLQGLSDPRGAIIRVENGLQALQPSLQVEPVKEEQTQ
AYLPTKQLQPTWSRSGGACGQQNGLDEIMCAFKLRKNIDNPQSSDIFNPHGGRITRANSQNFPILNIIQMSATRIVLQNNALLTPHWTVNAH
TVMYVTAGQGRIQVVDHRGRSVFDGELHQQQILLIPQNFAVVVKARREGFAWVSFKTNHNAVDSQIAGKASILRALPVDVVANAYRLSREDS
RRVKFNRGDEMAVFAPRRGPQQYAEWQINEK [SEQ ID 241]
>gi|573922051|ref|XP_006648611.1| PREDICTED: glutelin type-A 1-like [Oryza brachyantha]
MVDMSIVVPVCLTIFLLSQVCIAQVSFDGSPLYSSRGFRGGSASQQQCRFEHLAALEVTHQEKSEAGSIEYYNTEARDEFRCARVSARRLVIESR
GLVLPVYANAHKLLYIVQGRGVFGMALPGCPETFQSVRSAFEMATGDAESSTRKLRDEHQKIHQFRQGDVIAVPPGVAHWLYNNGDSPVVA
FSVIDFGNNANQLDPKPREFFLAGKPWGWQQVQYSYQSEQQSKHQNIFAGFNPDLLAEALSVSRQTAMRLQELNDQRGAIIRVEQGLQLAL
DPSFQAEQEQEEQPQEYLSSQQQQPTWSQRSGACVQNNGLDEIMCAFKVSKNINSAQSTDIFNPRGGRITRANSQNFPVLNIIQMSATRTV
LQNNALLTPHWTVNAHTVMYVTAGQGRIQVVDHRGRTVFDGELRQQQILLIPQNFAVAVKARHEGFSWVSFKTSHNAIDSQIAGKGSILRA
LPVDVLAKAYMLSREESRTLKYNRADETLVFAPRPEIQLYAESEK [SEQ ID 242]
Homologs of Pea protein 6 (SEQ ID 8)
>gi|164512534|emb|CAP06316.1| cvc [Pisum fulvum]
MATTTKSRFPLLLLLGIIFLASVVCVTYANYDEGSEPRVPGRRERGRQEGEKEEKRHGEWRPSYEKEEDEEEGQRERGRQEGEKEEKRHGEWG
PSYEKQEDEEEKQKYRYQREKEDEEEKQKYRYQREKKEQKEVQPGRERWEREEDEEHVDEEWRGSQRHEDPEERARLRYREERTKRDRRHQ
REGEEEERSSESQERRNPFLFKSNKFQTLFENENGHIRLLQRFDKRSDLFENLQNYRLVEYRAKPHTIFLPQHIDADLILVVLSGKAILTVLSPNAR
NSYNLERGDTIKLPAGTTSYLVNQDDEEDLRLVDLVIPVNGPGKFEAFDLSKNKNQYLRGFSKNILEASYNTKYETIEKVLLEEQEKTDAIVKVSRE
QIEELRKHAKSSSKKIFPSEFEPINLRNHKPEYSNKFGKLFEITPEKKYPQLQDLDIFVSCVEINEGALMLPHYNSRAIVVLLVNEGKGNLELLGLEN
EQQEREDRKERNNEVQRYEARLSPGDVVIIPAGHPVAITASSNLNLLAFGINAENNQRNFLSGSDDN [SEQ ID 243]
>gi|164512526|emb|CAP06312.1| cvc [Pisum abyssinicum]
MATTVESRFPLLLFPGIIFLASVCVTYANYDEGSETRVPGQRERGRQEGEKEEKRHGEWRPSYEKEEDEEEKQKYRYQREKEDEEEKQKYRYQR
EKKEEKEVQPGRERWEREEDEEQVDEEWRGSQRRQDPEERARLRHREERTKRDRRHKREGEEEERSSESQEQRNPFLFKSNKFLTLFENENG
HIRRLQRFDKRSDLFENLQNYRLVEYRAKPHTIFLPQHIDADLILVVLNGKAILTVLSPNDRNSYNLERGDTIKIPAGTTSYLVNQDDEEDLRVVD
FVIPVNRPGKFEAFGLSENKNQYLRGFSKNILEASLNTKYETIEKVLLEEQEKKPQQLRDRKRRQQGGERDAIIKVSREQIEELRKLAKSSSKKSLPS
EFEPFNLRSHKPEYSNKFGKLFEITPEKKYPQLQDLDILVSCVEINKGALMLPHYNSRAIVVLLVNEGKGNLELLGLKNEQQEREDRKERNNEVQ
RYEARLSPGDVVIIPAGHPVAISASSNLNLLGFGTNAENNQRNFLSGSDDN [SEQ ID 244]
>gi|164512558|emb|CAP06328.1| cvc [Vida villosa]
MATTIKSRFPVLLLLGIIFLTSVCVTYANYDEGREPSVPGQRERGRQEGEKEEKRHGEWRPSEEDEEEKYKYEEGRVPGQRERGRQEGEKEEKR
HGKWRPSEEEDEEEKYRYEEGSEPRGPGQRETGRQEGEKEKQRPEREPSYEKEEDEEEKQKYQYHREKKEQREVRPGRERFERHEDEEQWRG
IQRHEDPEERARERYRAEIAKRQVEEEREERDIPHEREQRNPFLFKSNKFQTLFQNENGYIRRLQRFDKRSDLFENLQNYRLVEYRAKPHTIFLPQ
HIDADLIIVVLSGRAILTVLSPDDRNSYNLERGDTIKLPAGTTSYLVNQDDEEDLRVVDLAIPVNRPGKVESFLLSGNKNQYLRGFSKNILEASFNT
NYETIERVLLEEQDKESQQSIGQKRRSQRQETNALVKVSREQLEDLKRLAKSSSQEGLSSQFEPINLRSQNPKYSNKFGKVFEITPEKKYPQLQDL
DLFVSSVDIKEGALMLPHYNSRAIVVLLVNEGRGNLELVGLKNEQQEQREKEDEQQERNNQVQRYEARLSPGDVVIIPAGHPVAVRASSDLNL
LAFGINAENNQRNFLAGSDDN [SEQ ID 245]
Homologs of Pea protein 7 (SEQ ID 9)
>gi|164512536|emb|CAP06317.1| cvc [Lathyrus hirsutus]
MAIIIKSRFPLLLLLGIIFLASVCATWANYDEGSEPRVPGQRERGRQEGEKAEKSHEKWRPSYEEEYDEGSEPRVPGKRERGRQEGEKEEKRHGE
WRPSHEEEYDEGSEPRVPTHGERGRQEGEKEEKRHEEWRPSYEKEEDEEEKEKYKYQREKKEQKEVQPGREKWERKQDEKHVEEDEDQEEE
QWRGSKRREDPEERARLRYREERTKSNVEEETEERRNPFLFKSNKFLTLFENENGHIRRLQRFDERSDIFENLQNYRLVEYKAKPHTMFLPQHID
ADLIIVVLNGKAILTVLSPNDRNSYNLERGDTIKLPAGTTSYLVNQDDEEDLRVVDLAIPVNRPGKFEAFGLSANKNQYLRGFSKNILEAFLNTKY
ETIEKVLLEEQERRDRKGRQQGQETNAIVKVSREQIEELRKLAKSSSKKSLLSESEPINLRSQNPKYSNKFGKLFEITPEKKYPQLQDLDVSISCVEI
NEGAPLLPHYNSRAIVLLLVNEGKGNLELVGFKNEQQRQRENEERNKKVQRYEARLSPGDVVVIPAGHPVAISASLNLNLVGFGVNAENNQR
NFLTGSDDN [SEQ ID 246]
>gi|164512542|emb|CAP06320.1| cvc [Lathyrus cicera]
MATIIKSRFPLLLLLGIIFLASVCVTLANYDEGSEPRVPAQRERGRQEGEKEEKRHGEWRPSHEKEYDEGSEPRVPGRRERGRQEGEKEEKRHGE
WRPSYEKEYDEGSEPRVPGRRERGRQEGEKEEKRHGEWRPSYEKEYDEEEKQKYQYEREKEEQKEVQPGRERWERKEDEEKEEDQWRGSQ
RHEDPEERARLRYRKERTKKYVEEDTEETSSESQGRRNPFLFKSNKFLTLFENENGYIRRLQRFDERSDIFENLQNYRLVEYRAKPHTIFLPQHIDA
DLILVILNGKAILTVLSPNDRNSYNLERGDTIKLPAGTTSYLVNEDDEEDLRVVDLVIPVNRPGKFEAFDLNQYLGGFSKSVLEASLNTKYETIEKVL
LEEQQKQGQETNAIVKVSREQIEELRKLAKSSSKKSLLSELEPVNLRSHSPKYSNKFGKFFEITPEKKYPQLQDLDVSISCVEINEGALLLPHYNSRA
IVVVLVNEGKGNLELLGVQNEDEQQERKERNKEVQRYEARLSPGDVVIIPSGHPVAVSASSNLNLLGFGINAENNQRNFLSGSDDN [SEQ ID
247]
>gi|164512544|emb|CAP06321.1| convicilin [Lathyrus sativus]
MATIIKSRFPLLLLLGIIFLASVCVTYANYDEGSEPRVPAQRERGRQEGEKEEKRHGEWRPSSEKEYDEGSEPRVPGRRERGRQEGEKEEKRHGE
WRPSYEKEYDEEEKQKYQYEREKKEQKEVEPGRERWERKEDEEKEEDQWRGSQRHEDPEERARLRYRKERTKKYVEEDTEETSSESQGRRNP
FLFKSNKFLTLFENENGYIRRLQRFDERSDLFENLQNYRLVEYRAKPHTIFLPQHIDADLILVILNGKAILTVLSPNDRNSYNLERGDTIKLPAGTTS
YLVNEDDEEDLRVVDLVIPVNRPGKFEAFDLNQYLGGFSKSVLKASLNTKYETIEKVLLEEQQKQGQETNAIVKVSREQIEELRKLAKSSSKKSLLS
ELEPVNLRSHSPKYSNKFGKFFEITPEKKYPQLQDLDVSISCVEINEGALLLPHYNSRAIVVLLVNEGKGNLELLGVQDEDEQQERKKRNKEVQRY
EARLSPSDVVIIPAGHPVAVSASSNLNLLGFGINAENNERNFLSGSDDN [SEQ ID 248]
Homologs of Rice protein 3 (SEQ ID 10)
>gi|573918992|ref|XP_006647120.1| PREDICTED: glutelin type-B 2-like [Oryza brachyantha]
MATTVFSRFSTYFCVLLLCHGSMAQLFNPSTNPWHNPRQGSSRECRFDRLQPFEPLRKVRSEAGVTEYFDEKNELFQCTGTFVIRRVIQPQGLL
VPRYTNAPGLVYIIQGRGSIGLTFPGCPATYQQQFQQFLPQEQSQSQKFRDEHQKIHQFRQGDIVALPAGVAHWFYNDGDAPVVAVYVYDV
KNSANQLEPRQREFLLGGNNMRAQQVYGSSAEQHSRQNIFSGFGVEILSEALGISTVTTKRLQSQNDQRGEIIHVKNGLQFLKPTLTQQQEQA
QAQYQEVQYSEQQQTSSRWNGLDENFCTIKARMNIENTSRADTYNPRAGRTTSLNSQKFPILNLVQMSATRVNLYQNAILSTFWNVNAHSL
VYTIQGRARVQVVSNFGKTVFDGELRPGQLLIIPQHYVVLKKAQREGFRYIAIKTNANAFVSQLVGKNSVFRSLPVDVIANVYRISREQARSLKN
NRGEEHGAFAPRSQQQSYPGFSNQSESETSE [SEQ ID 249]
>gi|573919041|ref|XP_006647142.1| PREDICTED: glutelin type-B 4-like [Oryza brachyantha]
MATTTFSRFSIYFCVLLLCHGSMAQLFSPTLNPWHSSRRGGSRDCRFDRLQAFEPLRRVRSEAGVTEYFDERNEQFQCTGTFVIRRVIEPQGLL
VPRYTNTPGVVYIMQGRGSMGLTFPGCPATYQQQFQQFLPEGQSQSQKFRDEHQKIHQFRQGDIVALPAGVAHWFYNEGDTPVVALYVFD
INNSANQLEPRQKDFLLAGNNNREQQVYGRSIEKHSGQNIFSGFNHELLSEALGISTLAAKRLQGQNDHRGEIIRVRNGLQLLKPTFTQQQEQ
AQSQYQVQYSEKQQESTRCNGLDENFCTINARLNIENPSRADTYNPRAGRITHLNNQKFPILNLVQMSATRVNLYQNAILSPYWNVNAHSLV
YMVQGHARVQVVSNLGKTVFNSVLRPGQLLIIPQHYVVLKKAEREGCQYIAFKTNANSIVSQLAGKNSILRAMPVDVVANAYRISREQARDLK
NNRGEELGAFTPKFEQQSYPGLSNESESEASE [SEQ ID 250]
>gi|109894635|gb|ABG47337.1| glutelin precursor [Zizania latifolia]
MNMATINGPTIFFTVCLFLLCHGSLAQLLGQSTSQWQSSHRGSSRQCRFDRLQAFEPVRSVRSQAGTTEFFDASNELFQCAGVSIVRRIIEPRG
LLLPQYTNGATIMYIIQGRGITGQTFPGCPESYQQQFQQSMQAQLTGSQSQSQKFKDEHQKINRFRQGDVIALPAGVAHWCYNDGEVPVVA
IYVIDINNAANQLDPRQRDFLLAGNMRSPQAYRREVENQSQNIFSGFSAELLSEALGISTGVARQLQCQNDQRGEIVRVEHGLSLLQPYASLQE
QEQKQEQPRERYQVTQHQQSQYGGGCSNGLDETFCAMRIWQNIDNPNLADTYNPRAGRVTNLNSQKFPILNLIQMSAVKVNLYQNALLSP
FWNINSHSVVYVTQGCARVQVVNNNGKTVFNGELRRGQLLIIPQHYVVVKKAQREGCAYIAFKTNPNSMVSH1VGKSSIFRALPTDVLANAY
RISREDAQRLKHNRGDELGAFTPLQYKSYQDVSSVAASS [SEQ ID 251]
Homologs of Rice protein 4 (SEQ ID 11)
>gi|531874314|gb|AGT59174.1| glutelin, partial [Oryza sativa Indica Group]
CRFDRLQAFEPIRSVRSQAGTTEFFDVSNEQFQCTGVSAVRRVIEPRGLLLPHYTNGASLVYIIQGRGITGPTFPGCPESYQQQFQQSGQAQLT
ESQSQSHKFKDEHQKIHRFRQGDVIALPAGVAHWCYNDGEVPVVAIYVTDLNNGANQLDPRQRDFLLAGNKRNPQAYRREVEERSQNIFSG
FSTELLSEALGVSSQVARQLQCQNDQRGEIVRVEHGLSLLQPYASLQEQEQGQVQSRERYQEGQYQQSQYGSGCSNGLDETFCTMKVRQNI
DNPNRADTYNPRAGRVTNLNTQNFPILNLVQMSAVKVNLYQNALLSPFWNINAHSVVYITQGRARVQVVNNNGKTVFNGELRRGQLLIIPQ
HYAVVKKAQREGCAYIAFKTNPNSMVSHIAGKSSIFRALPNDVLANAYRISREEAQRLKHNRGDEFGAFTPIQYKSYQDVYNAAESS [SEQ ID
252]
>gi|109894635|gb|ABG47337.1| glutelin precursor [Zizania latifolia]
MNMATINGPTIFFTVCLFLLCHGSLAQLLGQSTSQWQSSHRGSSRQCRFDRLQAFEPVRSVRSQAGTTEFFDASNELFQCAGVSIVRRIIEPRG
LLLPQYTNGATIMYIIQGRGITGQTFPGCPESYQQQFQQSMQAQLTGSQSQSQKFKDEHQKINRFRQGDVIALPAGVAHWCYNDGEVPVVA
IYVIDINNAANQLDPRQRDFLLAGNMRSPQAYRREVENQSQNIFSGFSAELLSEALGISTGVARQLQCQNDQRGEIVRVEHGLSLLQPYASLQE
QEQKQEQPRERYQVTQHQQSQYGGGCSNGLDETFCAMRIWQNIDNPNLADTYNPRAGRVTNLNSQKFPILNLIQMSAVKVNLYQNALLSP
FWNINSHSVVYVTQGCARVQVVNNNGKTVFNGELRRGQLLIIPQHYVVVKKAQREGCAYIAFKTNPNSMVSH1VGKSSIFRALPTDVLANAY
RISREDAQRLKHNRGDELGAFTPLQYKSYQDVSSVAASS [SEQ ID 253]
>gi|472867|emb|CAA52764.1| 11S globulin [Avena sativa]
MATTSFPSMLFYFCIFLLFHGSMAQLFGQSSTPWQSSRQGGLRGCRFDRLQAFEPLRQVRSQAGITEYFDEQNEQFRCTGVSVIRRVIEPQGL
VLPQYHNAPALVYILQGRGFTGLTFPGCPATFQQQFQPFDQSQFAQGQRQSQTIKDEHQRVQRFKQGDVVALPAGIVHWCYNDGDAPIVA
IYVFDVNNNANQLEPRQKEFLLAGNNKREQQSGNNIFSGLSVQLLSEALGISQQAAQRIQSQNDQRGEIIRVSQGLQFLKPIVSQQVPGEQQV
YQPIQTQEGQATQYQVGQSTQYQVGKSTPYQGGQSSQYQAGQSWDQSFNGLEENFCSLEARKNIENPQHADTYNPRAGRITRLNSKNFPIL
NIVQMSATRVNLYQNAILSPFWNINAHSVIYMIQGHARVQVVNNNGQTVFNDILRRGQLLIVPQHFVVLKKAEREGCQYISFKTNPNSMVSH
IAGKSSILRALPIDVLANAYRISRQEARNLKNNRGEEFGAFTPKLTQKGFQSYQDIEEGSSSPVRASE [SEQ ID 254]
Homologs of Rice protein 5 (SEQ ID 12)
>gi|225959|prf|11404367A glutelin
MASTNRPIVFFTVCLFLLCDGSLAQQLLGQSTSQWQSSRRGSPRGCRFDRLQAFEPIRSVRSQAGTTEFFDVSNELFQCTGVSVVRRVIEPRGL
LLPHYTNGASLVYIIQGRGITGPTFPGCPETYQQQFQQSGQAGLTESQSQSHKFKDEHQKIHRFRQGDVIALPAGVAHWCYNDCEVPVVAIYV
TDINNGANQLDPRQRDFLLAGNKRNPQAYRREVEEWSQNIFSGFSTELLSEAFGISNQVARQLQCQNDQKGEIVRVERGLSLLQPYASLQEQ
EQGQMQSREHYQEGGYQQSQYGSGCPNGLDETFCVNKVRQNIDNPNRADTYNPRAGRVTNLSQNFPILNLVQMSAVKVNLYQNTDTWIS
MGQEENALLSPFWN1NAHSIVYITQGRAQVQVLRRGQLLIVPQHYVVVKKAQREGCAYIAFKTNPNSMVSHIAGKSSIFRALPTDVLANAYRIS
REEAQRLKHNRGDEFGAFTPLQYKSYQDVYNVAESS [SEQ ID 255]
>gi|573943558|ref|XP_006654150.1| PREDICTED: glutelin type-A 3-like [Oryza brachyantha]
MKSSIVFSTICLVLLCHGSLAQLLSQSTSQWQSSRRGSPRQCRFDQLQAFEPIRTVRSQAGVTEFYDVSNELFQCTGVSVVRRVIEPRGLLLPHY
SNGATLVYIIQGRGITGPTFPGCPETYQQQFQQSGEAQPFEGQSHKFRDEHQKIHRFRQGDVVALPAGVAHWCYNDGEVPIVAIYVTDIYNS
ANQLDPRHRDFFLAGNNKVAQQLYRSEARENSKNIFGGFSVELLSEALGISRGVARQLQCQNDQRGEIVRVEHGLALLQPYASVQEQQQEQV
QSRDYEQTQYQQKQPQGSCSNGLDETFCTMRLRQNIDNPNLADTYNPKAGRITYLNGQKFPILNLVQMSAVKVNLYQNAVLSPFWNINAHS
VVYITQGRARVQVVNNNGKTVFDGELRQGQLLIIPQHHVVLKKAQREGCSYIALKTNPNSIVSHIAGKNSIFRALPGDVVTNAYRISREEAKRIK
HNRGDESGVFAPSHAYRSYQDMSVAA [SEQ ID 256]
>gi|721641733|ref|XP_010231907.1| PREDICTED: 12S seed storage globulin 1-like [Brachypodium distachyon]
MAHTSFSSFLSYFCLFLLFHGSMAQVLGQVSTWQSSRQGGSRDCSFDRLQA1EPVTQVRSQAGLTEYFDEQNEQFRCAGVFVIRRVIEPRGLL
LPRYHNTPGLVYILQGNGFVGLTFPGCPETFREQFQQFRQTQSTLGQSQCQSQKLGDVHQRVHQFTQGDVVALPTGVAHWIYNGGDAPVV
IVYVFDVNNNANQLEPRQKEFLLGGNYNGVLQYGQNIFSGFNAQLLSQAFGINEQTSQRIQNQNDGRGDIIRVDNGLQFLKPVVTQQQPEQ
PFMPIQHQTGQSSRNGLEENFCSLEPRQNIEDPNRADTYNPRAGSITRLNGQNFPILNLVQMSATRVNLQKNAILSPFWN1NAHSVVYVIQG
HALVQVVNNQGHNVFNGLLHRGQLLIIPQNYVVLKKAESEGYQYIAFKTNANSMVSHIAGKNSILRALPVDVIANAYRISRQEAQNLKNNRGE
ETGVLTPNFSQSTCQSYQTEDVQSLRPMSHWSE [SEQ ID 257]
Homologs of Rice protein 6 (SEQ ID 13)
>gi|169244463|gb|ACA50505.1| seed allergenic protein RAG2 [Oryza sativa Japonica Group]
MASNKVVFSALLLIIVSVLAATATMADHHKDQVVYSLGERCQPGMGYPMYSLPRCRAVVKRQCVGHGAPGGAVDEQLRQDCCRQLAAVD
DSWCRCSALNHMVGGIYRELGATDVGHPMAXVFPGCRRGDLERAAASLPAFCNVDIPNGTGGVCYWLGYPRTPRTGH [SEQ ID 258]
>gi|5777592|emb|CAA44001.1| low molecular weight globulin [Oryza sativa]
MASNKVVFSALLLIIVSVLRRDGTMADHHKDQVVYSLGERCQPGMGYPMYSLPRCRAVVKRQCVGHGAPGAVDEQLRQDCCRQLAAVDD
SWCRCSALNHMVGGIYRELGATDVGHPMAEVFPGCRRGDLERAAASLPAFCNVDIPNGTGGVCYWLGYPRTPRTGH [SEQ ID 259]
>gi|115471175|ref|NP_001059186.1| Os07g0214600 [Oryza sativa Japonica Group] >gi|23616954|dbj|BAC20657.1|
allergen RA16 [Oryza sativa Japonica Group] >gi|113610722|dbj|BAF21100.1| Os07g0214600 [Oryza sativa
Japonica Group] >gi|125557687|gb|EAZ03223.1| hypothetical protein OsI_25372 [Oryza sativa Indica Group]
MASNKVVISALLVVVVSVLAATTTMADHHQEQVVYTPGQLCQPGIGYPTYPLPRCRAFVKRQCVAPGTVDEQVRRGCCRQLAAIDSSWCRC
DALNHMLRIIYRESGAADAGHPMAEVFRGCRRGDIERAAASLPAFCNVDIPNGVGGVCYWLPGTGY [SEQ ID 260]
Homologs of Rice protein 7 (SEQ ID 14)
>gi|115445309|ref|NP_001046434.1| Os02g0248800 [Oryza sativa Japonica Group] >gi|37993738|gb|AAR06952.1|
glutelin type-B [Oryza sativa Japonica Group] >gi|47497729|dbj|BAD19794.1| glutelin type-B [Oryza sativa
Japonica Group] >gi|113535965|dbj|BAF08348.1| Os02g0248800 [Oryza sativa Japonica Group] >gi|215768942|
dbj|BAH01171.1| unnamed protein product [Oryza sativa Japonica Group] >gi|284431772|gb|ADB84627.1|
glutelin [Oryza sativa Japonica Group]
MTISVFSRFSIYFCVLLLCNGSMAQLFDPATNQWQTHRQGSFRECRFERLQAFEPLQNVRSEAGVTEYFDETNELFQCTGTFVIRRVIQPQGLL
IPRYANTPGMVYIIQGRGSMGLTFPGCPATYQQQSQQFLFQGESQSQKFIDEHQKIHQFRQGDIVVLPTGVAHWFYNDGDTPVVALYVYDI
NNSANQLEPRHREFLLAGKNNRVQQVYGRSIQQHSGQNIFNGFSVEPLSEALNINTVTTKRLQSQNDQRGEIIHVKNGLQLLKPTLTQRQEQE
QAQYQEVQYSEKPQTSSRWNGLEENLCTIKTRLNIENPSRADSYDPRAGRITSLDSQKFPILNIIQMSATRVNLYQNAILTPFWNVNAHSLMYV
IRGRARVQVVSNFGKTVFDGVLRPEQLLIIPQNYVVLKKAQHEGCQYIAINTNANAFVSHLAGVDSVFHALPVDVIANAYCISREEARRLKNNR
GDEYGPFPPRLQQQIYPEFSNESKGETSE [SEQ ID 261]
>gi|428674402|gb|AFZ41188.1| glutelin, partial [Oryza sativa Japonica Group]
LLCHGSMAQIFSLG1NPWQNPRQGGSRECRFDRLQAFEPLRKVRHEAGVTEYFDEKNEQFQCTGTLVIRRIIEPQGLLLPRYSNTPGLVYIIQGT
GVLGLTFPGCPATYQKQFRHFGLEGGSQRQGKKLRDENQKIHQFRQGDVVALPSGIPHWFYNEGDTPVVALFVFDVNNNANQLEPRQKEFL
LAGNNIEQQVSNPSINKHSGQNIFNGFNTKLLSEALGVNIEVTRRLQSQNDRRGDIIRVKNGLRLIKPTITQQQEQTQDQYQQIQYHREQRSTS
KYNGLDENFCAIRARLNIENPNHADTYNPRAGRITNLNSQKFSILNLVQMSATRVNLYQNAILSPFWNINAHSLVYTIQGRARVQVVSNHGKA
VFNGVLRPGQLLIIPQNYVVMKKAELEGFQFIAFKTNPNAMVNHIAGKNSVLRAMPVDVIANAYRISRQEARSLKNNRGEEIGAFTPRYQQQ
KIHQEYSNPNESETQ [SEQ ID 262]
>gi|226510|prf|1515394A seed storage globulin
MATTRFPSLLFYSCIFLLCNGSMAQLFGQSFTPWQSSRQGGLRGCRFDRLQAFEPLRQVRSQAGITEYFDEQNEQFRCAGVSVIRRVIEPQGLL
LPQYHNAPGLVYILQGRGFTGLTFPGCPATFQQQFQPFDQARFAQGQSKSQNLKDEHQRVHHIKQGDVVALPAGIVHWCYNDGDAPIVAV
YVFDVNNNANQLEPRQKEFLLAGNNKREQQFGQNIFSGFSVQLLSEALGISQQAAQKIQSQNDQRGEIIRVSQGLQFLKPFVSQQGPVEHQA
YQPIQSQQEQSTQYQVGQSPQYQEGQSTQYQSGQSWDQSFNGLEENFCSLEARQNIENPKRADTYNPRAGRITHLNSKNFPTLNLVQMSA
TRVNLYQNAILSPYWNINAHSVMHMIQGRARVQVVNNHGQTVFNDILRRGQLLIIPQHYVVLKKAEREGCQYISFKTTPNSMVSYIAGKTSIL
RALPVDVLANAYRISRQESQNLKNNRGEEFGAFTPKFAQTGSQSYQDEGESSSTEKASE [SEQ ID 263]
Homologs of Rice protein 8 (SEQ ID 15)
>gi|83375868|gb|ABC17777.1| waxy [Oryza rufipogon]
MSALTTSQLATSATGFGIADRSAPSSLLRHGFQGLKPRSPAGGDATSLSVTTSARATPKQQRSVQRGSRRFPSVVVYATGAGMNVVFVGAEM
APWSKTGGLGDVLGGLPPAMAANGHRVMVISPRYDQYKDAWDTSVVAEIKVADRYERVRFFHCYKRGVDRVFVDHPSFLEKVWGKTGEKI
YGPDTGVDYKDNQMRFSLLCQAPRILNLNNNPYFKGTYGEDVVFVCNDWHTGPLASYLKNNYQPNGIYRNAKVAFCIHNISYQGRFAFEDYP
ELNLSERFRSSFDFIDGYDTPVEGRKINWMKAGILEADRVLTVSPYYAEELISGIARGCELDNIMRLTGITGIVNGMDVSEWDPSKDKYITAKYD
ATTAIEAKALNKEALQAEAGLPVDRKIPLIAFIGRLEEQKGPDVMAAAIPELMQEDVQIVLLGTGKKKFEKLLKSMEEKYPGKVRAVVKFNAPLA
HLIMAGADVLAVPSRFEPCGLIQLQGMRYGTPCACASTGGLVDTVIEGKTGFHMGRLSVDCKVVEPSDVKKVAATLKRAIKVVGTPAYEEMV
RNCMNQDLSWKGPAKNWENVLLGLGVAGSAPGIEGDEIAPLAKENVAAP [SEQ ID 264]
>gi|297614332|gb|ADI48504.1| glycogen synthetase [Oryza officinalis]
MSALTTSQLATSATGFGIADRSAPSSLLRHGFQGLKPRSPAGGDASSLSVTTSARATPKQQRSVQRGSRRFPSVVVYATGAGMNVVFVGAEM
APWSKTGGLGDVLGGLPPAMAANGHRVMVISPRHDQYKDAWDTSVVAEIKVADRYERVRFFHCYKRGVDRVFIDHPSFLEKVWGKTGEKI
YGPDTGVDYKDNQMRFSLLCQAALEAPRILNLNNNPYFKGTYGEDVVFVCNDWHTGPLPSYLKNNYQPNGIYRNAKVAFCIHNISYQGRFAF
EDYPELNLSERFRSSFDFIDGYDTPVEGRKINWMKAGILESDRVLTVSPYYAEELISGIARGCELDNIMRLTGITGIVNGMDVSEWDPSKDKYIA
AKYDATTAIEAKALNKEALQAEAGLPVDRKIPLIAFIGRLEEQKGPDVMAAAIPELMQENVQIVLLGTGKKKFEKLLKSMEEKYPGKVRAVVKF
NAPLAHLIMAGADVLAVPSRFEPCGLIQLQGMRYGTPCACASTGGLVDTVIEGKTGFHMGRLSVDCKVVEPSDVQKVATTLKRAIKIVGTPAY
NEMVRNCMNQDLSWKGPAKNWENVLLGLGVAGSAPGVEGEEIAPLAKENVAAP [SEQ ID 265]
>gi|389620054|gb|AFK93486.1| granule-bound starch synthase [Hordeum vulgare subsp. vulgare]
MAALATSQLATSGTVLGVTDRFRRPGFQGLRPRNPADAALGMRTIGASAAPKQSRKAHRGSRRCLSVVVRATGSGMNLVFVGAEMAPWS
KTGGLGDVLGGLPPAMAANGHRVMVVSPRYDQYKDAWDTSVISEIKVADEYERVRFFHCYKRGVDRVFIDHPWFLEKVRGKTKEKIYGPDA
GTDYEDNQQRFSLLCQAALEAPRILNLNNNPYFSGPYGEDVVFVCNDWHTGLLACYLKSNYQSNGIYRTAKVAFCIHNISYQGRFSFDDFAQL
NLPDRFKSSFDFIDGYDKPVEGRKINWMKAGILQADKVLTVSPYYAEELISDEARGCELDNIMRLTGITGIVNGMDVSEWDPTKDKFLAVNYDI
TTALEAKALNKEALQAEVGLPVDRKVPLVAFIGRLEEQKGPDVMIAAIPEILKEEDVQIILLGTGKKKFEKLLKSMEEKFPGKVRAVVRFNAPLAH
QMMAGADLLAVTSRFEPCGLIQLQGMRYGTPCVCASTGGLVDTIVEGKTGFHMGRLSVDCNVVEPADVKKVATTLKRAVKVVGTPAYQEM
VKNCMIQDLSWKGPAKNWEDVLLELGVEGSEPGIVGEEIAPLAMENVAAP [SEQ ID 266]
Homologs of GLUC (Staphylococcus aureus) protein 1 (SEQ ID 16)
>gi|446599182|ref|WP_000676528.1| glutamyl endopeptidase [Staphylococcus aureus] >gi|253729369|
gb|EES98098.1| trypsin [Staphylococcus aureus subsp. aureus TCH130] >gi|341844549|gb|EGS85761.1| glutamyl
endopeptidase [Staphylococcus aureus subsp. aureus 21259] >gi|537390486|gb|AGU61109.1| Glutamyl
endopeptidase precursor [Staphylococcus aureus subsp. aureus CN1] >gi|564714561|gb|ETD14665.1| glutamyl
endopeptidase [Staphylococcus aureus subsp. aureus KPL184] >gi|577466329|gb|EUG79766.1| glutamyl
endopeptidase [Staphylococcus aureus M0139]
>gi|580560623|gb|EVF84961.1| glutamyl endopeptidase [Staphylococcus aureus C0A56020]
>gi|580687002|gb|EVH10169.1| glutamyl endopeptidase [Staphylococcus aureus UCIM6080]
>gi|751815683|gb|KIN24957.1| glutamyl endopeptidase [Staphylococcus aureus MRSA_CVM43477]
>gi|781884797|dbj|BAR08486.1| glutamyl endopeptidase precursor [Staphylococcus aureus subsp. aureus]
>gi|781887762|dbj|BAR11210.1| glutamyl endopeptidase precursor [Staphylococcus aureus subsp. aureus]
MKGKFLKVSSLFVATLTTATLVSSPAANALSSKAMDNHPQQSQSSKQQTPKIQKGGNLKPLEQREHANVILPNNDRHQITDTTNGHYAPVTYI
QVEAPTGTFIASGVVVGKDTLLTNKHVVDATHGDPHALKAFPSAINQDNYPNGGFTAEQITKYSGEGDLAIVKFSPNEQNKHIGEVVKPATMS
NNAETQVNQNITVTGYPGDKPVATMWESKGKITYLKGEAMQYDLSTTGGNSGSPVFNEKNEVIGIHWGGVPNEFNGAVFINENVRNFLKQ
NIEDIHFANDDQPNNPDNPDNPNNPDNPNNPDEPNNPDNPNNPDNPDNGDNNNSDNPDAA [SEQ ID 267]

What is claimed is:

1. A natural peptide having 7 to 50 amino acids and comprising a fragment of a protein of SEQUENCE ID NO: 353 selected from SEQUENCE ID NO: 423 or 424, or an anti-inflammatory variant of the fragment having at least 70% sequence homology with the fragment.

2. A natural peptide of claim 1 in which the peptide has anti-inflammatory activity.

3. A natural peptide of claim 1 comprising SEQUENCE ID NO: 423 or SEQUENCE ID NO: 424.

4. A natural peptide of claim 1 consisting of SEQUENCE ID NO: 423 or SEQUENCE ID NO: 424.

5. A natural peptide of claim 1 comprising an anti-inflammatory variant of SEQUENCE ID NO: 423 having at least 70% sequence homology with SEQUENCE ID NO: 423.

6. A natural peptide of claim 1 comprising an anti-inflammatory variant of SEQUENCE ID NO: 424 having at least 70% sequence homology with SEQUENCE ID NO: 424.

7. A natural peptide according to claim 1 which is a modified peptide.

8. A natural peptide according to claim 7 which is a modified to incorporate a protecting group selected from a N-terminal protecting group, C-terminal protecting group, and a side-chain protecting group.

9. A composition comprising the peptide of claim 1.

10. A composition according to claim 9 and comprising SEQUENCE ID NO: 423 or SEQUENCE ID NO: 424.

11. A composition according to claim 9 and having a peptide profile comprising substantially all of the peptides of SEQUENCE ID NO: 108-109, 112-220, and 423 and 424.

12. A composition according to claim 9 in which the composition is a powder.

13. A composition according to claim 9 in which the composition is enriched in peptides having a molecular weight of less than 10 KD.

14. A food or beverage, nutritional supplement, personal care composition, or pharmaceutical composition, comprising a composition of claim 9.

15. A non-therapeutic method of preventing or inhibiting inflammation in a mammal comprising a step of topically administering to the mammal a natural peptide of claim 1.

16. (canceled)

17. A non-therapeutic method of preventing or inhibiting inflammation in a mammal comprising a step of topically administering to the mammal a composition of claim 9.

18. (canceled)

19. A non-therapeutic method of maintaining gut health in a mammal comprising a step of orally administering to the mammal a natural peptide of claim 1.

20. (canceled)

21. A non-therapeutic method of maintaining gut health in a mammal comprising a step of orally administering to the mammal a composition of claim 9.

22. (canceled)

23. A natural peptide of claim 1, for use in a method for treatment or prevention of inflammation or an inflammatory disorder in a mammal.

24. (canceled)

25. A natural peptide of claim 1, for use of claim 23, in which the inflammatory disorder is selected from a skin inflammatory disorder, an inflammatory disorder of the joints, an inflammatory disorder of the cardiovascular system, an inflammatory disorder of the lung or airways, and a gut inflammatory disorder.


SEQUENCE LISTING (425 sequences)

SEQ ID NO: 1 - protein, 459 aa, Pisum sativum
MAATTMKASFPLLMLMGISFLASVCVSSRSDPQNPFIFKSNKFQTLFENENGHIRLLQKFDQRSKIFENLQNYRLLEYKSKPHTIFLPQHTDADYILVVLSGKAILTVLKPDDRNSFNLERGDTIKLPAGTIAYLVNRDDNEELRVLDLAIPVNRPGQLQSFLLSGNQNQQNYLSGFSKNILEASFNTDYEEIEKVLLEEHEKETQHRRSLKDKRQQSQEENVIVKLSRGQIEELSKNAKSTSKKSVSSESEPFNLRSRGPIYSNEFGKFFEITPEKNPQLQDLDIFVNSVEIKEGSLLLPHYNSRAIVIVTVNEGKGDFELVGQRNENQQEQRKEDDEEEEQGEEEINKQVQNYKAKLSSGDVFVIPAGHPVAVKASSNLDLLGFGINAENNQRNFLAGDEDNVISQIQRPVKELAFPGSAQEVDRILENQKQSHFADAQPQQRERGSRETRDRLSSV

SEQ ID NO: 2 - protein, 124 aa, Pisum sativum
DRRQELSNENVLVKVSRRQLEELSKNAKSSSRRSVSSESGPFNLRSEDPLYSNNSGKFFELTPEKNQQLQDLDLFVNSVDLKEGSLLLPNYNSRALLVLVLVVNEGKGDFELVGQRNENQGKEN

SEQ ID NO: 3 - protein, 520 aa, Pisum sativum
MATKLLALSLSFCFLLLGGCFALREQPEQNECQLERLNALEPDNRIESEGGLIETWNPNNKQFRCAGVALSRATLQHNALRRPYYSNAPQEIFIQQGNGYFGMVFPGCPETFEEPQESEQGEGRRYRDRHQKVNRFREGDIIAVPTGIVFWMYNDQDTPVIAVSLTDIRSSNNQLDQMPRRFYLAGNHEQEFLRYQHQQGGKQEQENEGNNIFSGFKRDFLEDAFNVNRHIVDRLQGRNEDEEKGAIVKVKGGLSIISPPEKQARHQRGSRQEEDEDEDEERQPRHQRGSRQEEEEDEDEERQPRHQRRRGEEEEEDKKERRGSQKGKSRRQGDNGLEETVCTAKLRLNIGPSSSPDIYNPEAGRIKTVTSLDLPVLRWLKLSAEHGSLHKNAMFVPHYNLNANSIIYALKGRARLQVVNCNGNTVFDGELEAGRALTVPQNYAVAAKSLSDRFSYVAFKTNDRAGIARLAGTSSVINNLPLDVVAATFNLQRNEARQLKSNNPFKFLVPARQSENRASA

SEQ ID NO: 4 - protein, 613 aa, Pisum sativum
MATTIKSRFPLLLLLGIIFLASVVCVTYANYDEGSEPRVPAQRERGRQEGEKEEKRHGEWRPSYEKEEDEEEGQRERGRQEGEKEEKRHGEWRPSYEKQEDEEEKQKYRYQREKEDEEEKQKYQYQREKKEQKEVQPGRERWEREEDEEQVDEEWRGSQRREDPEERARLRHREERTKRDRRHQREGEEEERSSESQERRNPFLFKSNKFLTLFENENGHIRLLQRFDKRSDLFENLQNYRLVEYRAKPHTIFLPQHIDADLILVVLSGKAILTVLSPNDRNSYNLERGDTIKLPAGTTSYLVNQDDEEDLRLVDLVIPVNGPGKFEAFDLAKNKNQYLRGFSKNILEASYNTRYETIEKVLLEEQEKDRKRRQQGEETDAIVKVSREQIEELKKLAKSSSKKSLPSEFEPINLRSHKPEYSNKFGKLFEITPEKKYPQLQDLDLFVSCVEINEGALMLPHYNSRAIVVLLVNEGKGNLELLGLKNEQQEREDRKERNNEVQRYEARLSPGDVVIIPAGHPVAITASSNLNLLGFGINAENNERNFLSGSDDNVISQIENPVKELTFPGSVQEINRLIKNQKQSHFANAEPEQKEQGSQGKRSPLSSILGTFY

SEQ ID NO: 5 - protein, 438 aa, Pisum sativum
MAATPIKPLMLLAIAFLASVCVSSRSDQENPFIFKSNRFQTLYENENGHIRLLQKFDKRSKIFENLQNYRLLEYKSKPRTLFLPQYTDADFILVVLSGKATLTVLKSNDRNSFNLERGDTIKLPAGTIAYLANRDDNEDLRVLDLTIPVNKPGQLQSFLLSGTQNQPSLLSGFSKNILEAAFNTNYEEIEKVLLEQQEQEPQHRRSLKDRRQEINEENVIVKVSREQIEELSKNAKSSSKKSVSSESGPFNLRSRNPIYSNKFGKFFEITPEKNQQLQDLDIFVNSVDIKEGSLLLPNYNSRAIVIVTVTEGKGDFELVGQRNENQGKENDKEEEQEEETSKQVQLYRAKLSPGDVFVIPAGHPVAINASSDLNLIGFGINAENNERNFLAGEEDNVISQVERPVKELAFPGSSHEVDRLLKNQKQSYFANAQPLQRE

SEQ ID NO: 6 - protein, 484 aa, Oryza sativa
MATTTSLLSSCLCALLLAPLFSQGVDAWESRQGASRQCRFDRLQAFEPLRKVRSEAGDTEYFDERNEQFRCAGVFVIRRVIEPQGLVVPRYSNTPALAYIIQGKGYVGLTFPGCPATHQQQFQLFEQRQSDQAHKFRDEHQKIHEFRQGDVVALPASVAHWFYNGGDTPAVVVYVYDIKSFANQLEPRQKEFLLAGNNQRGQQIFEHSIFQHSGQNIFSGFNTEVLSEALGINTEASKRLQSQNDQRGDIIRVKHGLQLLKPTLTQRQEEHRQYQQVQYREGQYNGLDENFCTIKARVNIENPSRADYYNPRAGRITLLNNQKFPILNLIGMGAARVNLYQNALLSPFWNINAHSVVYIIQGSVRVQVANNQGRSVFNGVLHQGQLLIIPQNHAVIKKAEHNGCQYVAIKTISDPTVSWVAGKNSILRALPVDVIANAYRISRDEARRLKNNRADEIGPFTPRFPQKSQRGYQFLTEGLSLIGM

SEQ ID NO: 7 - protein, 510 aa, Oryza sativa
MASMSTILPLCLGLLLFFQVSMAQFSFGGSPLQSPRGFRGDQDSRHQCRFEHLTALEATHQQRSEAGFTEYYNIEARNEFRCAGVSVRRLVVESKGLVLPMYANAHKLVYIVQGRGVFGMALPGCPETFQSVRSPFEQEVATAGEAQSSIQKMRDEHQQLHQFHQGDVIAVPAGVAHWLYNNGDSPVVAFTVIDTSNNANQLDPKRREFFLAGKPRSSWQQQSYSYQTEQLSRNQNIFAGFSPDLLSEALSVSKQTVLRLQGLSDPRGAIIRVENGLQALQPSLQVEPVKEEQTQAYLPTKQLQPTWLRSGGACGQQNVLDEIMCAFKLRKNIDNPQSSDIFNPHGGRITRANSQNFPILNIIQMSATRIVLQNNALLTPHWTVNAHTVMYVTAGQGHIQVVDHRGRSVFDGELHQQQILLIPQNFAVVVKARREGFAWVSFKTNHNAVDSQIAGKASILRALPVDVVANAYRLSREDSRHVKFNRGDEMAVFAPRRGPQQYAEWQINEK

SEQ ID NO: 8 - protein, 386 aa, Pisum sativum
MATTIKSRFPLLLLLGIIFLASVVSVTYANYDEGSEPRVPAQRERGRQEGEKEEKRHGEWRPSYEKEEDEEEGQRERGRQEGEKEEKRHGEWGPSYEKQEDEEEKQKYRYQREKEDEEEKQKYQYQREKKEQKEVQPGRERWEREEDEEQVDEEWRGSQRREDPEERARLRHREERTKRDRRHQREGEEEERSSESQERRNPFLFKSNKFLTLFENENGHIRLLQRFDKRSDLFENLQNYRLVEYRAKPHTIFLPQHIDADLILVVLSGKAILTVLSPNDRNSYNLERGDTIKLPAGTTSYLVNQDDEEDLRLVDLVIPVNGPGKFEAFDLAKNKNQYLRGFSKNILEASYNTRYETIEKVLLEEQEKDRKRRQQGEETDAIVKVS

SEQ ID NO: 9 - protein, 386 aa, Pisum sativum; identical to SEQ ID NO: 8

SEQ ID NO: 10 - protein, 499 aa, Oryza sativa
MASSVFSRFSIYFCVLLLCHGSMAQLFNPSTNPWHSPRQGSFRECRFDRLQAFEPLRKVRSEAGVTEYFDEKNELFQCTGTFVIRRVIQPQGLLVPRYTNIPGVVYIIQGRGSMGLTFPGCPATYQQQFQQFSSQGQSQSQKFRDEHQKIHQFRQGDIVALPAGVAHWFYNDGDAPIVAVYVYDVNNNANQLEPRQKEFLLAGNNNRAQQQQVYGSSIEQHSGQNIFSGFGVEMLSEALGINAVAAKRLQSQNDQRGEIIHVKNGLQLLKPTLTQQQEQAQAQDQYQQVQYSERQQTSSRWNGLEENFCTIKVRVNIENPSRADSYNPRAGRITSVNSQKFPILNLIQMSATRVNLYQNAILSPFWNVNAHSLVYMIQGRSRVQVVSNFGKTVFDGVLRPGQLLIIPQHYAVLKKAEREGCQYIAIKTNANAFVSHLAGKNSVFRALPVDVVANAYRISREQARSLKNNRGEEHGAFTPRFQQQYYPGLSNESESETSE

SEQ ID NO: 11 - protein, 499 aa, Oryza sativa
MASINRPIVFFTVCLFLLCNGSLAQQLLGQSTSQWQSSRRGSPRECRFDRLQAFEPIRSVRSQAGTTEFFDVSNEQFQCTGVSVVRRVIEPRGLLLPHYTNGASLVYIIQGRGITGPTFPGCPESYQQQFQQSGQAQLTESQSQSQKFKDEHQKIHRFRQGDVIALPAGVAHWCYNDGEVPVVAIYVTDLNNGANQLDPRQRDFLLAGNKRNPQAYRREVEERSQNIFSGFSTELLSEALGVSSQVARQLQCQNDQRGEIVRVEHGLSLLQPYASLQEQEQGQVQSRERYQEGQYQQSQYGSGCSNGLDETFCTLRVRQNIDNPNRADTYNPRAGRVTNLNTQNFPILSLVQMSAVKVNLYQNALLSPFWNINAHSVVYITQGRARVQVVNNNGKTVFNGELRRGQLLIIPQHYAVVKKAQREGCAYIAFKTNPNSMVSHIAGKSSIFRALPNDVLANAYRISREEAQRLKHNRGDEFGAFTPIQYKSYQDVYNAAESS

SEQ ID NO: 12 - protein, 499 aa, Oryza sativa
MASINRPIVFFTVCLFLLCDGSLAQQLLGQSTSQWQSSRRGSPRGCRFDRLQAFEPIRSVRSQAGTTEFFDVSNELFQCTGVSVVRRVIEPRGLLLPHYTNGASLVYIIQGRGITGPTFPGCPETYQQQFQQSGQAQLTESQSQSHKFKDEHQKIHRFRQGDVIALPAGVAHWCYNDGEVPVVAIYVTDINNGANQLDPRQRDFLLAGNKRNPQAYRREVEEWSQNIFSGFSTELLSEAFGISNQVARQLQCQNDQRGEIVRVERGLSLLQPYASLQEQEQGQMQSREHYQEGGYQQSQYGSGCPNGLDETFCTMRVRQNIDNPNRADTYNPRAGRVTNLNSQNFPILNLVQMSAVKVNLYQNALLSPFWNINAHSIVYITQGRAQVQVVNNNGKTVFNGELRRGQLLIVPQHYVVVKKAQREGCAYIAFKTNPNSMVSHIAGKSSIFRALPTDVLANAYRISREEAQRLKHNRGDEFGAFTPLQYKSYQDVYNVAESS

SEQ ID NO: 13 - protein, 160 aa, Oryza sativa
MASNKVVFSVLLLAVVSVLAATATMAEYHHQDQVVYTPGPLCQPGMGYPMYPLPRCRALVKRQCVGRGTAAAAEQVRRDCCRQLAAVDDSWCRCEAISHMLGGIYRELGAPDVGHPMSEVFRGCRRGDLERAAASLPAFCNVDIPNGGGGVCYWLARSGY

SEQ ID NO: 14 - protein, 500 aa, Oryza sativa
MATIAFSRLSIYFCVLLLCHGSMAQLFGPNVNPWHNPRQGGFRECRFDRLQAFEPLRRVRSEAGVTEYFDEKNEQFQCTGTFVIRRVIEPQGLLVPRYSNTPGMVYIIQGRGSMGLTFPGCPATYQQQFQQFLPEGQSQSQKFRDEHQKIHQFRQGDIVALPAGVAHWFYNEGDAPVVALYVFDLNNNANQLEPRQKEFLLAGNNNREQQMYGRSIEQHSGQNIFSGFNNELLSEALGVNALVAKRLQGQNDQRGEIIRVKNGLKLLRPAFAQQQEQAQQQEQAQAQYQVQYSEEQQPSTRCNGLDENFCTIKARLNIENPSHADTYNPRAGRITRLNSQKFPILNLVQLSATRVNLYQNAILSPFWNVNAHSLVYIVQGHARVQVVSNLGKTVFNGVLRPGQLLIIPQHYVVLKKAEHEGCQYISFKTNANSMVSHLAGKNSIFRAMPVDVIANAYRISREQARSLKNNRGEELGAFTPRYQQQTYPGFSNESENEALE

SEQ ID NO: 15 - protein, 609 aa, Oryza sativa
MSALTTSQLATSATGFGIADRSAPSSLLRHGFQGLKPRSPAGGDATSLSVTTSARATPKQQRSVQRGSRRFPSVVVYATGAGMNVVFVGAEMAPWSKTGGLGDVLGGLPPAMAANGHRVMVISPRYDQYKDAWDTSVVAEIKVADRYERVRFFHCYKRGVDRVFIDHPSFLEKVWGKTGEKIYGPDTGVDYKDNQMRFSLLCQAALEAPRILNLNNNPYFKGTYGEDVVFVCNDWHTGPLASYLKNNYQPNGIYRNAKVAFCIHNISYQGRFAFEDYPELNLSERFRSSFDFIDGYDTPVEGRKINWMKAGILEADRVLTVSPYYAEELISGIARGCELDNIMRLTGITGIVNGMDVSEWDPSKDKYITAKYDATTAIEAKALNKEALQAEAGLPVDRKIPLIAFIGRLEEQKGPDVMAAAIPELMQEDVQIVLLGTGKKKFEKLLKSMEEKYPGKVRAVVKFNAPLAHLIMAGADVLAVPSRFEPCGLIQLQGMRYGTPCACASTGGLVDTVIEGKTGFHMGRLSVDCKVVEPSDVKKVAATLKRAIKVVGTPAYEEMVRNCMNQDLSWKGPAKNWENVLLGLGVAGSAPGIEGDEIAPLAKENVAAP

SEQ ID NO: 16 - protein, 336 aa, Staphylococcus aureus
MKGKFLKVSSLFVATLTTATLVSSPAANALSSKAMDNHPQQTQSSKQQTPKIQKGGNLKPLEQREHANVILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDEPNNPDNPNNPDNPDNGDNNNSDNPDAA

SEQ ID NO: 17 - protein, 22 aa, synthetic construct
  REGION 1..22: note = Synthetic peptide
PFNLRSRGPIYSNEFGKFFEIT

SEQ ID NO: 18 - protein, 17 aa, synthetic construct
  REGION 1..17: note = Synthetic peptide
KPDDRNSFNLERGDTIK

SEQ ID NO: 19 - protein, 27 aa, synthetic construct
  REGION 1..27: note = Synthetic peptide
YLVNRDDNEELRVLDLAIPVNRPGQLQ

SEQ ID NO: 20 - protein, 51 aa, synthetic construct
  REGION 1..51: note = Synthetic peptide
QEQRKEDDEEEEQGEEEINKQVQNYKAKLSSGDVFVIPAGHPVAVKASSNL

SEQ ID NO: 21 - protein, 68 aa, synthetic construct
  REGION 1..68: note = Synthetic peptide
SGFSKNILEASFNTDYEEIEKVLLEEHEKETQHRRSLKDKRQQSQEENVIVKLSRGQIEELSKNAKST

SEQ ID NO: 22 - protein, 74 aa, synthetic construct
  REGION 1..74: note = Synthetic peptide
PFIFKSNKFQTLFENENGHIRLLQKFDQRSKIFENLQNYRLLEYKSKPHTIFLPQHTDADYILVVLSGKAILTV

SEQ ID NO: 23 - protein, 28 aa, synthetic construct
  REGION 1..28: note = Synthetic peptide
QEVDRILENQKQSHFADAQPQQRERGSR

SEQ ID NO: 24 - protein, 35 aa, synthetic construct
  REGION 1..35: note = Synthetic peptide
NNSGKFFELTPEKNQQLQDLDLFVNSVDLKEGSLL

SEQ ID NO: 25 - protein, 17 aa, synthetic construct
  REGION 1..17: note = Synthetic peptide
KVSRRQLEELSKNAKSS

SEQ ID NO: 26 - protein, 21 aa, synthetic construct
  REGION 1..21: note = Synthetic peptide
QRGSRQEEDEDEDEERQPRHQ

SEQ ID NO: 27 - protein, 33 aa, synthetic construct
  REGION 1..33: note = Synthetic peptide
QEFLRYQHQQGGKQEQENEGNNIFSGFKRDFLE

SEQ ID NO: 28 - protein, 34 aa, synthetic construct
  REGION 1..34: note = Synthetic peptide
YNLERGDTIKLPAGTTSYLVNQDDEEDLRLVDLV

SEQ ID NO: 29 - protein, 80 aa, synthetic construct
  REGION 1..80: note = Synthetic peptide
NGPGKFEAFDLAKNKNQYLRGFSKNILEASYNTRYETIEKVLLEEQEKDRKRRQQGEETDAIVKVSREQIEELKKLAKSS

SEQ ID NO: 30 - protein, 47 aa, synthetic construct
  REGION 1..47: note = Synthetic peptide
RYEARLSPGDVVIIPAGHPVAITASSNLNLLGFGINAENNERNFLSG

SEQ ID NO: 31 - protein, 24 aa, synthetic construct
  REGION 1..24: note = Synthetic peptide
HGEWRPSYEKQEDEEEKQKYRYQR

SEQ ID NO: 32 - protein, 19 aa, synthetic construct
  REGION 1..19: note = Synthetic peptide
PSYEKEEDEEEGQRERGRQ

SEQ ID NO: 33 - protein, 30 aa, synthetic construct
  REGION 1..30: note = Synthetic peptide
KSKPRTLFLPQYTDADFILVVLSGKATLTV

SEQ ID NO: 34 - protein, 22 aa, synthetic construct
  REGION 1..22: note = Synthetic peptide
SQSQKFRDEHQKIHQFRQGDIV

SEQ ID NO: 35 - protein, 23 aa, synthetic construct
  REGION 1..23: note = Synthetic peptide
VNSQKFPILNLIQMSATRVNLYQ

SEQ ID NO: 36 - protein, 40 aa, synthetic construct
  REGION 1..40: note = Synthetic peptide
YIAIKTNANAFVSHLAGKNSVFRALPVDVVANAYRISREQ

SEQ ID NO: 37 - protein, 31 aa, synthetic construct
  REGION 1..31: note = Synthetic peptide
APIVAVYVYDVNNNANQLEPRQKEFLLAGNN

SEQ ID NO: 38 - protein, 17 aa, synthetic construct
  REGION 1..17: note = Synthetic peptide
ENPSRADSYNPRAGRIT

SEQ ID NO: 39 - protein, 32 aa, synthetic construct
  REGION 1..32: note = Synthetic peptide
GLQLLKPTLTQQQEQAQAQDQYQQVQYSERQQ

SEQ ID NO: 40 - protein, 18 aa, synthetic construct
  REGION 1..18: note = Synthetic peptide
CRFDRLQAFEPLRKVRSE

SEQ ID NO: 41 - protein, 48 aa, synthetic construct
  REGION 1..48: note = Synthetic peptide
ILSPFWNVNAHSLVYMIQGRSRVQVVSNFGKTVFDGVLRPGQLLIIPQ

SEQ ID NO: 42 - protein, 24 aa, synthetic construct
  REGION 1..24: note = Synthetic peptide
AFTPRFQQQYYPGLSNESESETSE

SEQ ID NO: 43 - protein, 31 aa, synthetic construct
  REGION 1..31: note = Synthetic peptide
PRAGRVTNLNTQNFPILSLVQMSAVKVNLYQ

SEQ ID NO: 44 - protein, 32 aa, synthetic construct
  REGION 1..32: note = Synthetic peptide
HSVVYITQGRARVQVVNNNGKTVFNGELRRGQ

SEQ ID NO: 45 - protein, 32 aa, synthetic construct
  REGION 1..32: note = Synthetic peptide
HIAGKSSIFRALPNDVLANAYRISREEAQRLK

SEQ ID NO: 46 - protein, 29 aa, synthetic construct
  REGION 1..29: note = Synthetic peptide
VPVVAIYVTDLNNGANQLDPRQRDFLLAG

SEQ ID NO: 47 - protein, 52 aa, synthetic construct
  REGION 1..52: note = Synthetic peptide
TFCTMRVRQNIDNPNRADTYNPRAGRVTNLNSQNFPILNLVQMSAVKVNLYQ

SEQ ID NO: 48 - protein, 73 aa, synthetic construct
  REGION 1..73: note = Synthetic peptide
YIAFKTNPNSMVSHIAGKSSIFRALPTDVLANAYRISREEAQRLKHNRGDEFGAFTPLQYKSYQDVYNVAESS

SEQ ID NO: 49 - protein, 36 aa, synthetic construct
  REGION 1..36: note = Synthetic peptide
SQSHKFKDEHQKIHRFRQGDVIALPAGVAHWCYNDG

SEQ ID NO: 50 - protein, 29 aa, synthetic construct
  REGION 1..29: note = Synthetic peptide
TQGRAQVQVVNNNGKTVFNGELRRGQLLI

SEQ ID NO: 51 - protein, 24 aa, synthetic construct
  REGION 1..24: note = Synthetic peptide
NGASLVYIIQGRGITGPTFPGCPE

SEQ ID NO: 52 - protein, 21 aa, synthetic construct
  REGION 1..21: note = Synthetic peptide
CRFDRLQAFEPIRSVRSQAGT

SEQ ID NO: 53 - protein, 40 aa, synthetic construct
  REGION 1..40: note = Synthetic peptide
QNDQRGEIVRVERGLSLLQPYASLQEQEQGQMQSREHYQE

SEQ ID NO: 54 - protein, 35 aa, synthetic construct
  REGION 1..35: note = Synthetic peptide
PRQRDFLLAGNKRNPQAYRREVEEWSQNIFSGFST

SEQ ID NO: 55 - protein, 23 aa, synthetic construct
  REGION 1..23: note = Synthetic peptide
GGIYRELGAPDVGHPMSEVFRGC

SEQ ID NO: 56 - protein, 26 aa, synthetic construct
  REGION 1..26: note = Synthetic peptide
HSLVYIVQGHARVQVVSNLGKTVFNG

SEQ ID NO: 57 - protein, 23 aa, synthetic construct
  REGION 1..23: note = Synthetic peptide
ARSLKNNRGEELGAFTPRYQQQT

SEQ ID NO: 58 - protein, 23 aa, synthetic construct
  REGION 1..23: note = Synthetic peptide
LNSQKFPILNLVQLSATRVNLYQ

SEQ ID NO: 59 - protein, 84 aa, synthetic construct
  REGION 1..84: note = Synthetic peptide
QMYGRSIEQHSGQNIFSGFNNELLSEALGVNALVAKRLQGQNDQRGEIIRVKNGLKLLRPAFAQQQEQAQQQEQAQAQYQVQYS

SEQ ID NO: 60 - protein, 24 aa, synthetic construct
  REGION 1..24: note = Synthetic peptide
AQLFGPNVNPWHNPRQGGFRECRF

SEQ ID NO: 61 - protein, 23 aa, synthetic construct
  REGION 1..23: note = Synthetic peptide
GVAHWFYNEGDAPVVALYVFDLN

SEQ ID NO: 62 - protein, 40 aa, synthetic construct
  REGION 1..40: note = Synthetic peptide
YISFKTNANSMVSHLAGKNSIFRAMPVDVIANAYRISREQ

SEQ ID NO: 63 - protein, 38 aa, synthetic construct
  REGION 1..38: note = Synthetic peptide
KGPAKNWENVLLGLGVAGSAPGIEGDEIAPLAKENVAA

SEQ ID NO: 64 - protein, 31 aa, synthetic construct
  REGION 1..31: note = Synthetic peptide
RAVVKFNAPLAHLIMAGADVLAVPSRFEPCG

SEQ ID NO: 65 - protein, 22 aa, synthetic construct
  REGION 1..22: note = Synthetic peptide
KRAIKVVGTPAYEEMVRNCMNQ

SEQ ID NO: 66 - protein, 55 aa, synthetic construct
  REGION 1..55: note = Synthetic peptide
APWSKTGGLGDVLGGLPPAMAANGHRVMVISPRYDQYKDAWDTSVVAEIKVADRY

SEQ ID NO: 67 - protein, 109 aa, synthetic construct
  REGION 1..109: note = Synthetic peptide
INWMKAGILEADRVLTVSPYYAEELISGIARGCELDNIMRLTGITGIVNGMDVSEWDPSKDKYITAKYDATTAIEAKALNKEALQAEAGLPVDRKIPLIAFIGRLEEQK

SEQ ID NO: 68 - protein, 45 aa, synthetic construct
  REGION 1..45: note = Synthetic peptide
RGVDRVFIDHPSFLEKVWGKTGEKIYGPDTGVDYKDNQMRFSLLC

SEQ ID NO: 69 - protein, 21 aa, synthetic construct
  REGION 1..21: note = Synthetic peptide
LEAPRILNLNNNPYFKGTYGE

SEQ ID NO: 70 - protein, 26 aa, synthetic construct
  REGION 1..26: note = Synthetic peptide
YIQVEAPTGTFIASGVVVGKDTLLTN

SEQ ID NO: 71 - protein, 12 aa, synthetic construct
  REGION 1..12: note = Synthetic peptide
SRGPIYSNEFGK

SEQ ID NO: 72 - protein, 7 aa, synthetic construct
  REGION 1..7: note = Synthetic peptide
NSFNLER

SEQ ID NO: 73 - protein, 10 aa, synthetic construct
  REGION 1..10: note = Synthetic peptide
VLDLAIPVNR

SEQ ID NO: 74 - protein, 7 aa, synthetic construct
  REGION 1..7: note = Synthetic peptide
DDNEELR

SEQ ID NO: 75 - protein, 18 aa, synthetic construct
  REGION 1..18: note = Synthetic peptide
LSSGDVFVIPAGHPVAVK

SEQ ID NO: 76 - protein, 15 aa, synthetic construct
  REGION 1..15: note = Synthetic peptide
EDDEEEEQGEEEINK

SEQ ID NO: 77 - protein, 29 aa, synthetic construct
  REGION 1..29: note = Synthetic peptide
NILEASFNTDYEEIEKVLLEEHEKETQHR

SEQ ID NO: 78 - protein, 24 aa, synthetic construct
  REGION 1..24: note = Synthetic peptide
NILEASFNTDYEEIEKVLLEEHEK

SEQ ID NO: 79 - protein, 16 aa, synthetic construct
  REGION 1..16: note = Synthetic peptide
NILEASFNTDYEEIEK

SEQ ID NO: 80 - protein, 12 aa, synthetic construct
  REGION 1..12: note = Synthetic peptide
RQQSQEENVIVK

SEQ ID NO: 81 - protein, 11 aa, synthetic construct
  REGION 1..11: note = Synthetic peptide
QQSQEENVIVK

SEQ ID NO: 82 - protein, 11 aa, synthetic construct
  REGION 1..11: note = Synthetic peptide
LSRGQIEELSK

SEQ ID NO: 83 - protein, 8 aa, synthetic construct
  REGION 1..8: note = Synthetic peptide
GQIEELSK

SEQ ID NO: 84 - protein, 8 aa, synthetic construct
  REGION 1..8: note = Synthetic peptide
VLLEEHEK

SEQ ID NO: 85 - protein, 24 aa, synthetic construct
  REGION 1..24: note = Synthetic peptide
SKPHTIFLPQHTDADYILVVLSGK

SEQ ID NO: 86 - protein, 22 aa, synthetic construct
  REGION 1..22: note = Synthetic peptide
PHTIFLPQHTDADYILVVLSGK

SEQ ID NO: 87 - protein, 16 aa, synthetic construct
  REGION 1..16: note = Synthetic peptide
SNKFQTLFENENGHIR

SEQ ID NO: 88 - protein, 11 aa, synthetic construct
  REGION 1..11: note = Synthetic peptide
SKIFENLQNYR

SEQ ID NO: 89 - protein, 9 aa, synthetic construct
  REGION 1..9: note = Synthetic peptide
IFENLQNYR

SEQ ID NO: 90 - protein, 18 aa, synthetic construct
  REGION 1..18: note = Synthetic peptide
ILENQKQSHFADAQPQQR

SEQ ID NO: 91 - protein, 24 aa, synthetic construct
  REGION 1..24: note = Synthetic peptide
PGQLQSFLLSGNQNQQNYLSGFSK

SEQ ID NO: 92 - protein, 25 aa, synthetic construct
  REGION 1..25: note = Synthetic peptide
FFELTPEKNQQLQDLDLFVNSVDLK

SEQ ID NO: 93 - protein, 7 aa, synthetic construct
  REGION 1..7: note = Synthetic peptide
QLEELSK

SEQ ID NO: 94 - protein, 11 aa, synthetic construct
  REGION 1..11: note = Synthetic peptide
QEEDEDEDEER

SEQ ID NO: 95 - protein, 23 aa, synthetic construct
  REGION 1..23: note = Synthetic peptide
YQHQQGGKQEQENEGNNIFSGFK

SEQ ID NO: 96 - protein, 24 aa, synthetic construct
  REGION 1..24: note = Synthetic peptide
GDTIKLPAGTTSYLVNQDDEEDLR

SEQ ID NO: 97 - protein, 12 aa, synthetic construct
  REGION 1..12: note = Synthetic peptide
RQQGEETDAIVK

SEQ ID NO: 98 - protein, 11 aa, synthetic construct
  REGION 1..11: note = Synthetic peptide
VLLEEQEKDRK

SEQ ID NO: 99 - protein, 10 aa, synthetic construct
  REGION 1..10: note = Synthetic peptide
NILEASYNTR

SEQ ID NO: 100 - protein, 8 aa, synthetic construct
  REGION 1..8: note = Synthetic peptide
FEAFDLAK

SEQ ID NO: 101 - protein, 8 aa, synthetic construct
  REGION 1..8: note = Synthetic peptide
EQIEELKK

SEQ ID NO: 102 - protein, 7 aa, synthetic construct
  REGION 1..7: note = Synthetic peptide
EQIEELK

SEQ ID NO: 103 - protein, 7 aa, synthetic construct
  REGION 1..7: note = Synthetic peptide
NKNQYLR

SEQ ID NO: 104 - protein, 37 aa, synthetic construct
  REGION 1..37: note = Synthetic peptide
LSPGDVVIIPAGHPVAITASSNLNLLGFGINAENNER

SEQ ID NO: 105 - protein, 14 aa, synthetic construct
  REGION 1..14: note = Synthetic peptide
PSYEKQEDEEEKQK

SEQ ID NO: 106 - protein, 9 aa, synthetic construct
  REGION 1..9: note = Synthetic peptide
EEDEEEGQR

SEQ ID NO: 107 - protein, 20 aa, synthetic construct
  REGION 1..20: note = Synthetic peptide
TLFLPQYTDADFILVVLSGK

SEQ ID NO: 108 - protein, 24 aa, synthetic construct
  REGION 1..24: note = Synthetic peptide
GYVGLTFPGCPATHQQQFQLFEQR

SEQ ID NO: 109 - protein, 14 aa, synthetic construct
  REGION 1..14: note = Synthetic peptide
RGPQQYAEWQINEK

SEQ ID NO: 110 - protein, 21 aa, synthetic construct
  REGION 1..21: note = Synthetic peptide
LVDLVIPVNGPGKFEAFDLAK

SEQ ID NO: 111 - protein, 18 aa, synthetic construct
  REGION 1..18: note = Synthetic peptide
KNPQLQDLDIFVNYVEIK

SEQ ID NO: 112 - protein, 10 aa, synthetic construct
  REGION 1..10: note = Synthetic peptide
DEHQKIHQFR

SEQ ID NO: 113 - protein, 7 aa, synthetic construct
  REGION 1..7: note = Synthetic peptide
FRDEHQK

SEQ ID NO: 114 - protein, 13 aa, synthetic construct
  REGION 1..13: note = Synthetic peptide
FPILNLIQMSATR

SEQ ID NO: 115 - protein, 13 aa, synthetic construct
  REGION 1..13: note = Synthetic peptide
TNANAFVSHLAGK

SEQ ID NO: 116 - protein, 12 aa, synthetic construct
  REGION 1..12: note = Synthetic peptide
ALPVDVVANAYR

SEQ ID NO: 117 - protein, 20 aa, synthetic construct
  REGION 1..20: note = Synthetic peptide
YVYDVNNNANQLEPRQKEFL

SEQ ID NO: 118 - protein, 21 aa, synthetic construct
  REGION 1..21: note = Synthetic peptide
VYVYDVNNNANQLEPRQKEFL

SEQ ID NO: 119 - protein, 7 aa, synthetic construct
  REGION 1..7: note = Synthetic peptide
ADSYNPR

SEQ ID NO: 120 - protein, 16 aa, synthetic construct
  REGION 1..16: note = Synthetic peptide
KPTLTQQQEQAQAQDQ

SEQ ID NO: 121 - protein, 13 aa, synthetic construct
  REGION 1..13: note = Synthetic peptide
QAQAQDQYQQVQY

SEQ ID NO: 122 - protein, 11 aa, synthetic construct
  REGION 1..11: note = Synthetic peptide
QAQDQYQQVQY

SEQ ID NO: 123 - protein, 8 aa, synthetic construct
  REGION 1..8: note = Synthetic peptide
LQAFEPLR

SEQ ID NO: 124 - protein, 11 aa, synthetic construct
  REGION 1..11: note = Synthetic peptide
SRVQVVSNFGK

SEQ ID NO: 125 - protein, 10 aa, synthetic construct
  REGION 1..10: note = Synthetic peptide
WNVNAHSLVY

SEQ ID NO: 126 - protein, 9 aa, synthetic construct
  REGION 1..9: note = Synthetic peptide
NVNAHSLVY

SEQ ID NO: 127 - protein, 15 aa, synthetic construct
  REGION 1..15: note = Synthetic peptide
IQGRSRVQVVSNFGK

SEQ ID NO: 128 - protein, 14 aa, synthetic construct
  REGION 1..14: note = Synthetic peptide
GKTVFDGVLRPGQL

SEQ ID NO: 129 - protein, 15 aa, synthetic construct
  REGION 1..15: note = Synthetic peptide
FGKTVFDGVLRPGQL

SEQ ID NO: 130 - protein, 19 aa, synthetic construct
  REGION 1..19: note = Synthetic peptide
FQQQYYPGLSNESESETSE

SEQ ID NO: 131 - protein, 9 aa, synthetic construct
  REGION 1..9: note = Synthetic peptide
QQYYPGLSN

SEQ ID NO: 132 - protein, 10 aa, synthetic construct
  REGION 1..10: note = Synthetic peptide
QQQYYPGLSN

SEQ ID NO: 133 - protein, 21 aa, synthetic construct
  REGION 1..21: note = Synthetic peptide
VTNLNTQNFPILSLVQMSAVK

SEQ ID NO: 134 - protein, 19 aa, synthetic construct
  REGION 1..19: note = Synthetic peptide
ITQGRARVQVVNNNGKTVF

SEQ ID NO: 135 - protein, 22 aa, synthetic construct
  REGION 1..22: note = Synthetic peptide
ITQGRARVQVVNNNGKTVFNGE

SEQ ID NO: 136 - protein, 21 aa, synthetic construct
  REGION 1..21: note = Synthetic peptide
ITQGRARVQVVNNNGKTVFNG

SEQ ID NO: 137 - protein, 13 aa, synthetic construct
  REGION 1..13: note = Synthetic peptide
RVQVVNNNGKTVF

SEQ ID NO: 138 - protein, 18 aa, synthetic construct
  REGION 1..18: note = Synthetic peptide
RALPNDVLANAYRISREE

SEQ ID NO: 139 - protein, 16 aa, synthetic construct
  REGION 1..16: note = Synthetic peptide
SIFRALPNDVLANAYR

SEQ ID NO: 140 - protein, 15 aa, synthetic construct
  REGION 1..15: note = Synthetic peptide
SIFRALPNDVLANAY

SEQ ID NO: 141 - protein, 13 aa, synthetic construct
  REGION 1..13: note = Synthetic peptide
SIFRALPNDVLAN

SEQ ID NO: 142 - protein, 17 aa, synthetic construct
  REGION 1..17: note = Synthetic peptide
SSIFRALPNDVLANAYR

SEQ ID NO: 143 - protein, 21 aa, synthetic construct
  REGION 1..21: note = Synthetic peptide
SIFRALPNDVLANAYRISREE

SEQ ID NO: 144 - protein, 9 aa, synthetic construct
  REGION 1..9: note = Synthetic peptide
SIFRALPND

SEQ ID NO: 145 - protein, 19 aa, synthetic construct
  REGION 1..19: note = Synthetic peptide
IYVTDLNNGANQLDPRQRD

SEQ ID NO: 146 - protein, 21 aa, synthetic construct
  REGION 1..21: note = Synthetic peptide
VTNLNSQNFPILNLVQMSAVK

SEQ ID NO: 147 - protein, 8 aa, synthetic construct
  REGION 1..8: note = Synthetic peptide
QNIDNPNR

SEQ ID NO: 148 - protein, 7 aa, synthetic construct
  REGION 1..7: note = Synthetic peptide
ADTYNPR

SEQ ID NO: 149 - protein, 21 aa, synthetic construct
  REGION 1..21: note = Synthetic peptide
NIDNPNRADTYNPRAGRVTNL

SEQ ID NO: 150 - protein, 25 aa, synthetic construct
  REGION 1..25: note = Synthetic peptide
RVRQNIDNPNRADTYNPRAGRVTNL

SEQ ID NO: 151 - protein, 18 aa, synthetic construct
  REGION 1..18: note = Synthetic peptide
TNPNSMVSHIAGKSSIFR

SEQ ID NO: 152 - protein, 16 aa, synthetic construct
  REGION 1..16: note = Synthetic peptide
HNRGDEFGAFTPLQYK

SEQ ID NO: 153 - protein, 12 aa, synthetic construct
  REGION 1..12: note = Synthetic peptide
SYQDVYNVAESS

SEQ ID NO: 154 - protein, 8 aa, synthetic construct
  REGION 1..8: note = Synthetic peptide
ISREEAQR

SEQ ID NO: 155 - protein, 21 aa, synthetic construct
  REGION 1..21: note = Synthetic peptide
SIFRALPTDVLANAYRISREE

SEQ ID NO: 156 - protein, 19 aa, synthetic construct
  REGION 1..19: note = Synthetic peptide
YRISREEAQRLKHNRGDEF

SEQ ID NO: 157 - protein, 18 aa, synthetic construct
  REGION 1..18: note = Synthetic peptide
YRISREEAQRLKHNRGDE

SEQ ID NO: 158 - protein, 10 aa, synthetic construct
  REGION 1..10: note = Synthetic peptide
FKDEHQKIHR

SEQ ID NO: 159 - protein, 14 aa, synthetic construct
  REGION 1..14: note = Synthetic peptide
QGDVIALPAGVAHW

SEQ ID NO: 160 - protein, 9 aa, synthetic construct
  REGION 1..9: note = Synthetic peptide
TVFNGELRR

SEQ ID NO: 161 - protein, 8 aa, synthetic construct
  REGION 1..8: note = Synthetic peptide
TVFNGELR

SEQ ID NO: 162 - protein, 13 aa, synthetic construct
  REGION 1..13: note = Synthetic peptide
QVQVVNNNGKTVF

SEQ ID NO: 163 - protein, 13 aa, synthetic construct
  REGION 1..13: note = Synthetic peptide
YIIQGRGITGPTF

SEQ ID NO: 164 - protein, 14 aa, synthetic construct
  REGION 1..14: note = Synthetic peptide
VYIIQGRGITGPTF

SEQ ID NO: 165 - protein, 11 aa, synthetic construct
  REGION 1..11: note = Synthetic peptide
LQAFEPIRSVR

SEQ ID NO: 166 - protein, 22 aa, synthetic construct
  REGION 1..22: note = Synthetic peptide
GLSLLQPYASLQEQEQGQMQSR

SEQ ID NO: 167 - protein, 8 aa, synthetic construct
  REGION 1..8: note = Synthetic peptide
GEIVRVER

SEQ ID NO: 168 - protein, 13 aa, synthetic construct
  REGION 1..13: note = Synthetic peptide
RGLSLLQPYASLQ

SEQ ID NO: 169 - protein, 15 aa, synthetic construct
  REGION 1..15: note = Synthetic peptide
RGLSLLQPYASLQEQ

SEQ ID NO: 170 - protein, 16 aa, synthetic construct
  REGION 1..16: note = Synthetic peptide
RGLSLLQPYASLQEQE

SEQ ID NO: 171 - protein, 14 aa, synthetic construct
  REGION 1..14: note = Synthetic peptide
RGLSLLQPYASLQE

SEQ ID NO: 172 - protein, 7 aa, synthetic construct
  REGION 1..7: note = Synthetic peptide
RNPQAYR

SEQ ID NO: 173 - protein, 13 aa, synthetic construct
  REGION 1..13: note = Synthetic peptide
FLLAGNKRNPQAY

SEQ ID NO: 174 - protein, 10 aa, synthetic construct
  REGION 1..10: note = Synthetic peptide
EVEEWSQNIF

SEQ ID NO: 175 - protein, 12 aa, synthetic construct
  REGION 1..12: note = Synthetic peptide
LAGNKRNPQAYR

SEQ ID NO: 176 - protein, 12 aa, synthetic construct
  REGION 1..12: note = Synthetic peptide
FLLAGNKRNPQA

SEQ ID NO: 177 - protein, 13 aa, synthetic construct
  REGION 1..13: note = Synthetic peptide
ELGAPDVGHPMSE

SEQ ID NO: 178 - protein, 16 aa, synthetic construct
  REGION 1..16: note = Synthetic peptide
IVQGHARVQVVSNLGK

SEQ ID NO: 179 - protein, 14 aa, synthetic construct
  REGION 1..14: note = Synthetic peptide
IVQGHARVQVVSNL

SEQ ID NO: 180 - protein, 13 aa, synthetic construct
  REGION 1..13: note = Synthetic peptide
IVQGHARVQVVSN

SEQ ID NO: 181 - protein, 13 aa, synthetic construct
  REGION 1..13: note = Synthetic peptide
NNRGEELGAFTPR

SEQ ID NO: 182 - protein, 10 aa, synthetic construct
  REGION 1..10: note = Synthetic peptide
GEELGAFTPR

SEQ ID NO: 183 - protein, 13 aa, synthetic construct
  REGION 1..13: note = Synthetic peptide
FPILNLVQLSATR

SEQ ID NO: 184 - protein, 31 aa, synthetic construct
  REGION 1..31: note = Synthetic peptide
SIEQHSGQNIFSGFNNELLSEALGVNALVAK

SEQ ID NO: 185 - protein, 8 aa, synthetic construct
  REGION 1..8: note = Synthetic peptide
LQGQNDQR

SEQ ID NO: 186 - protein, 20 aa, synthetic construct
  REGION 1..20: note = Synthetic peptide
SGFNNELLSEALGVNALVAK

SEQ ID NO: 187 - protein, 20 aa, synthetic construct
  REGION 1..20: note = Synthetic peptide
PAFAQQQEQAQQQEQAQAQY

SEQ ID NO: 188 - protein, 15 aa, synthetic construct
  REGION 1..15: note = Synthetic peptide
VAKRLQGQNDQRGEI

SEQ ID NO: 189 - protein, 17 aa, synthetic construct
  REGION 1..17: note = Synthetic peptide
ALVAKRLQGQNDQRGEI

SEQ ID NO: 190 - protein, 13 aa, synthetic construct
  REGION 1..13: note = Synthetic peptide
LQGQNDQRGEIIR

SEQ ID NO: 191 - protein, 14 aa, synthetic construct
  REGION 1..14: note = Synthetic peptide
PNVNPWHNPRQGGF

SEQ ID NO: 192 - protein, 13 aa, synthetic construct
  REGION 1..13: note = Synthetic peptide
FYNEGDAPVVALY

SEQ ID NO: 193 - protein, 10 aa, synthetic construct
  REGION 1..10: note = Synthetic peptide
FYNEGDAPVV

SEQ ID NO: 194 - protein, 12 aa, synthetic construct
  REGION 1..12: note = Synthetic peptide
FYNEGDAPVVAL

SEQ ID NO: 195 - protein, 11 aa, synthetic construct
  REGION 1..11: note = Synthetic peptide
FYNEGDAPVVA

SEQ ID NO: 196 - protein, 13 aa, synthetic construct
  REGION 1..13: note = Synthetic peptide
TNANSMVSHLAGK

SEQ ID NO: 197 - protein, 12 aa, synthetic construct
  REGION 1..12: note = Synthetic peptide
AMPVDVIANAYR

SEQ ID NO: 198 - protein, 28 aa, synthetic construct
  REGION 1..28: note = Synthetic peptide
NWENVLLGLGVAGSAPGIEGDEIAPLAK

SEQ ID NO: 199 - protein, 19 aa, synthetic construct
  REGION 1..19: note = Synthetic peptide
NVLLGLGVAGSAPGIEGDE

SEQ ID NO: 200 - protein, 28 aa, synthetic construct
  REGION 1..28: note = Synthetic peptide
NWENVLLGLGVAGSAPGIEGDEIAPLAK

SEQ ID NO: 201 - protein, 21 aa, synthetic construct
  REGION 1..21: note = Synthetic peptide
FNAPLAHLIMAGADVLAVPSR

SEQ ID NO: 202 - protein, 10 aa, synthetic construct
  REGION 1..10: note = Synthetic peptide
FNAPLAHLIM

SEQ ID NO: 203 - protein, 21 aa, synthetic construct
  REGION 1..21: note = Synthetic peptide
FNAPLAHLIMAGADVLAVPSR

SEQ ID NO: 204 - protein, 12 aa, synthetic construct
  REGION 1..12: note = Synthetic peptide
VVGTPAYEEMVR

SEQ ID NO: 205 - protein, 21 aa, synthetic construct
  REGION 1..21: note = Synthetic peptide
TGGLGDVLGGLPPAMAANGHR

SEQ ID NO: 206 - protein, 17 aa, synthetic construct
  REGION 1..17: note = Synthetic peptide
YDQYKDAWDTSVVAEIK

SEQ ID NO: 207 - protein, 12 aa, synthetic construct
  REGION 1..12: note = Synthetic peptide
DAWDTSVVAEIK

SEQ ID NO: 208 - protein, 7 aa, synthetic construct
  REGION 1..7: note = Synthetic peptide
VMVISPR

SEQ ID NO: 209 - protein, 22 aa, synthetic construct
  REGION 1..22: note = Synthetic peptide
LTGITGIVNGMDVSEWDPSKDK

SEQ ID NO: 210 - protein, 18 aa, synthetic construct
  REGION 1..18: note = Synthetic peptide
VLTVSPYYAEELISGIAR

SEQ ID NO: 211 - protein, 14 aa, synthetic construct
  REGION 1..14: note = Synthetic peptide
EALQAEAGLPVDRK

SEQ ID NO: 212 - protein, 10 aa, synthetic construct
  REGION 1..10: note = Synthetic peptide
YDATTAIEAK

SEQ ID NO: 213 - protein, 9 aa, synthetic construct
  REGION 1..9: note = Synthetic peptide
IPLIAFIGR

SEQ ID NO: 214 - protein, 8 aa, synthetic construct
  REGION 1..8: note = Synthetic peptide
AGILEADR

SEQ ID NO: 215 - protein, 9 aa, synthetic construct
  REGION 1..9: note = Synthetic peptide
IPLIAFIGR

SEQ ID NO: 216 - protein, 11 aa, synthetic construct
  REGION 1..11: note = Synthetic peptide
VFIDHPSFLEK

SEQ ID NO: 217 - protein, 13 aa, synthetic construct
  REGION 1..13: note = Synthetic peptide
GPDTGVDYKDNQM

SEQ ID NO: 218 - protein, 16 aa, synthetic construct
  REGION 1..16: note = Synthetic peptide
IYGPDTGVDYKDNQMR

SEQ ID NO: 219 - protein, 11 aa, synthetic construct
  REGION 1..11: note = Synthetic peptide
IYGPDTGVDYK

SEQ ID NO: 220 - protein, 11 aa, synthetic construct
  REGION 1..11: note = Synthetic peptide
ILNLNNNPYFK

SEQ ID NO: 221 - protein, 16 aa, synthetic construct
  REGION 1..16: note = Synthetic peptide
APTGTFIASGVVVGKD

SEQ ID NO: 222 - protein, 463 aa, Vicia faba
MAATTLKDSFPLLTLLGIAFLASVCLSSRSDQDNPFVFESNRFQTLFENENGHIRLLQKFDQHSKLLENLQNYRLLEYKSKPHTIFLPQQTDADFILVVLSGKAILTVLLPNDRNSFSLERGDTIKLPAGTIGYLVNRDDEEDLRVLDLVIPVNRPGEPQSFLLSGNQNQPSILSGFSKNILEASFNTDYKEIEKVLLEEHGKEKYHRRGLKDRRQRGQEENVIVKISRKQIEELNKNAKSSSKKSTSSESEPFNLRSREPIYSNKFGKFFEITPKRNPQLQDLNIFVNYVEINEGSLLLPHYNSRAIVIVTVNEGKGDFELVGQRNENQQGLREEYDEEKEQGEEEIRKQVQNYKAKLSPGDVLVIPAGYPVAIKASSNLNLVGFGINAENNQRYFLAGEEDNVISQIHKPVKELAFPGSAQEVDTLLENQKQSHFANAQPRERERGSQEIKDHLYSILGSF

SEQ ID NO: 223 - protein, 446 aa, Cicer arietinum
MAIKARFPLLVLLGIVFLASVCAKSDKENPFFFKSNNCQTLFENENGHVRLLQRFDKRSQLFENLQNYRLMEYNSKPHTLFLPQHNDADFILVVLRGRAILTVLNPNDRNTFKLERGDTIKLPAGTIAYLANRDDNEDLRVLDLAIPVNRPGQFQSFSLSGNENQQSYFQGFSKKILEASFNSDYEEIERVLLEEQEQKPEQRRGHKGRQQSQETDVIVKISREQIEELSKNAKSNCKKSVSSESEPFNLRSRSPIYSNRFGNFFEITPEKNPQLKDLDIFVNSVEIKEGSLLLPHFNSRATVILVVNEGKGEVELVGLRNENEQENKKEDEEEEEDRNVQVQRFQSKLSSGDVVVIPASHPFSINASSDLFLLGFGINAQNNQRNFLAGEEDNVISQIQRPVKEVAFPGSAEEVDRLLKNQRQSHFANAQPQQKRKGSQRIRSPF

SEQ ID NO: 224 - protein, 418 aa, Lens culinaris
SRSDQENPFIFKSNRFQTIYENENGHIRLLQRFDKRSKIFENLQNYRLLEYKSKPHTIFLPQFTDADFILVVLSGKAILTVLNSNDRNSFNLERGDTIKLPAGTIAYLANRDDNEDLRVLDLAIPVNRPGQLQSFLLSGTQNQPSFLSGFSKNILEAAFNTEYEEIEKVLLEEQEQKSQHRRSLRDKRQEITNEDVIVKVSREQIEELSKNAKSSSKKSVSSESEPFNLRSRNPIYSNKFGKFFEITPEKNPQLQDLDIFVNSVEIKEGSLLLPNYNSRAIVIVTVNEGKGDFELVGQRNENQQEQREENDEEEGQEEETTKQVQRYRARLSPGDVLVIPAGHPVAINASSDLNLIGFGINAKNNQRNFLAGEEDNVISQIQRPVKELAFPGSSREVDRLLTNQKQSHFANAQPLQIE

SEQ ID NO: 225 - protein, 415 aa, Lens culinaris
SRSDQENPFIFKSNRFQTIYENENGHIRLLQKFDKRSKIFENLQNYRLLEYKSKPHTLFLPQYTDADFILVVLSGKAVLTVLNSNDRNSFNLERGDTIKLPAGTIAYLANRDDNEDLRVLDLAIPVNNPGQLESFLLSGTQNQPSFLSGFNKSILEAAFNTDYEEIEKVLLEDQEQEPQHRRSLRDRRQEINKENVIVKVSREQIKELSKNAKSSSKKSVSSESEPFNLRSRNPIYSNKFGKFFEITPEKNPQLQDLDIFVNSVEIKEGSLLLPNYNSRAIVIVTVNEGKGYFELVGQRNENQREENDDEEEQEEETSTQVQRYRAKLSPGDVFVVPAGHPVAINASSDLNLIGFGINAKNNQRNFLAGEEDNVISQIQRPVKELAFPGSSREVDRLLTNQKQSHFANAQPLQIE

SEQ ID NO: 226 - protein, 463 aa, Vicia narbonensis
MAAITMKVSFPLLMLLGISFLASVCVSSRSDQENPFIFKSNKFQTLFENDNGHIRLLQKFDERSKILENLQNYRLLEYKSKPRTIFLPQQTNADFILVVLSGKAILTVLKPDDRNSFNLERGDTIKLPAGTIAYLVNKDDNEDLRVLDLAIPVNGPDQLQSFLLSGSENQQSILSGFSKSVLEASFNTGYEEIEKVLLEEREKETQHRRSLRDKRQHSQDEDVIVKLSRGQIEELSRNAKSSSKKSVSSESEPFNLRSRNPIYSNKFGKFFEITPEKNPQLQDLDVLVNSVEIKEGSLLLPHYNSRAIVIVTVNDGKGDFEIVGQRNENRQGQRKEDDEEEEQGDENTNTQVQNYKAKLSRGDVFVIPAGHPVSIKASSNLDLLGFGINAKNNQRNFLAGEEDNVISQIDRPVKELAFPGSAQEVDRLLENQKQSHFANAQPQQRERGSHETRDHLSSILDAF

SEQ ID NO: 227 - protein, 396 aa, Glycine max
QYGHVRVLQRFNKRSQQLQNLRDYRILEFNSKPNTLLLPHHADADYLIVILNGTAILTLVNNDDRDSYNLQSGDALRVPAGTTYYVVNPDNDENLRMITLAIPVNKPGRFESFFLSSTQAQQSYLQGFSKNILEASYDTKFEEINKVLFGREEGQQQGEERLQESVIVEISKKQIRELSKHAKSSSRKTISSEDKPFNLRSRDPIYSNKLGKLFEITPEKNPQLRDLDVFLSVVDMNEGALFLPHFNSKAIVVLVINEGEANIELVGIKEQQQRQQQEEQPLEVRKYRAELSEQDIFVIPAGYPVVVNATSDLNFFAFGINAENNQRNFLAGSKDNVISQIPSQVQELAFPGSAKDIENLIKSQSESYFVDAQPQQKEEGNKGRKGPLSSILRAFY

SEQ ID NO: 228 - protein, 498 aa, Vicia sativa
MAKLLALSLSFCFLLFSSCFALREQSQQNECQLERINALEPDNRIESEGGLIETWNPNNRQFRCARVALSRATLQRNALRRPYYSNAPQEIYIQQGNGYFGMVFPGCPETHEEPQQSEQGEGRRYRDSHQKVNRFREGDIIAVPTGIAFWMYNDQDTPVIAISLTDTGSSNNQLDQMPRRFYLAGNQEQEFLRYQHQQGGKQEQDNDGNNIFSGFKRDFLEDAFNVNRHIVDRLQGRNEDEEKGAIVKVKGGLSIIAPPERQARHERGSRQEEDEDEKEERQPSHHKSRRDEDEDDKEKRHSQKGQSRRQGDNGLEETVCTAKLRANIGSSPSPDIYNPQAGRIKTVTSLDLPVLRWLKLSAEHGSLHKNAMFVPHYNLNANSVIYALKGRARLQVVNCNGNTVFDGELEAGRALTVPQNYAVAAKSLSERFTYVAFKTDDRASIARLAGTSSVIDDLPLDVVAATFNMQRNEARQLKSNNPFKFLVPPRQSEMRASA

SEQ ID NO: 229 - protein, 531 aa, Medicago truncatula
MAKLLALSLSLCFLLFSGCFAIREHQPHQKQQPQQNECQLEQLNALEPDNRIESEGGIIETWNPNNRQFRCAGVALSRCTLQRNSLRRPFYSNAPQEIFIQQGSGYFGMVFPGCPETFEEPQESEQRESRRIRESEQGESRRIRESEQGEGRRFRDSHQKVNRFREGDLIAVPTGTVFWMYNDQDTPVIAVSLIDTGSFQNQLDEMPRRFYLAGNQEQEFLQYQQQQVRGRGEQRRGREQQENEGGNIFSGFKRDFLEDALNVNRHIVDRLQGRNEDEEKGAIVKVRGGLSFVTPPERQSRHQGGSIIEEDEDEEDEWRRPHHQKSRRGEEEERPCRRGQKCERSNGLEETICTARLRQNIGSSSSPDIYNPEAGRIKTVTSFDLPALRWLRLSAEHGTLHRNAMFVPHYNLNANSAIYALRGRARLQVVNCNGNTVFDGELEAGRVLIVPQNFAVAAKSMSDRFQYVSFKTNDNAAIARLAGTQSTLSGVPMDVLAATYNMDRNEARQLKNNNLYKFLVPPRESERRAAA

SEQ ID NO: 230 - protein, 476 aa, Lotus japonicus
MAYKLFALSLSFCFLLFGGCFAIRQQSQQQNECQLERLNALKPDNRIESEAGYIETWNPTNNQFRCAGVALSRCTLRRNGLKRPSYSNAPQEIFIQQGSGIFGMIFPGCPETVEEPFESDQQGRRDRHQKVNRFREGDVIAVPPGVVFWMYNEEETPVIAVSLIDTGSYLNQLDQMPRRFYLSGNQEQEFLQYQRQEVRGREEENQGGNIFSGFGGEFLEDALNIDRNIVHKLQGRDEEQDKGAIVRVKGGLSVITPPERQSHRRGSEEEEDEEEDRPSRHQSRGGSRRNGLEETICTVRLRMNIGKSSSPDIFNPQAGRIKTATGFDFPALRFLKLSAEHGSLNRNAMVVPHYNLNANSIIYALRGRAWIQVVNCKGNRIFDGELEEGQVLIVPQNFVVAARSMSDKFNYVAFKTNDMPTMAKLAGATSEIQAMPLEVIQNAFNLEREQAKQVKFNNRFNFLVPPREQSQRRASA

SEQ ID NO: 231 - protein, 526 aa, PISUM ABYSSINICUM
MATTVESRFPLLLFPGIIFLASVCVTYANYDEGSETRVPGQRERGRQEGEKEEKRHGEWRPSYEKEEDEEEKQKYRYQREKEDEEEKQKYRYQREKKEEKEVQPGRERWEREEDEEQVDEEWRGSQRRQDPEERARLRHREERTKRDRRHKREGEEEERSSESQEQRNPFLFKSNKFLTLFENENGHIRRLQRFDKRSDLFENLQNYRLVEYRAKPHTIFLPQHIDADLILVVLNGKAILTVLSPNDRNSYNLERGDTIKIPAGTTSYLVNQDDEEDLRVVDFVIPVNRPGKFEAFGLSENKNQYLRGFSKNILEASLNTKYETIEKVLLEEQEKKPQQLRDRKRRQQGGERDAIIKVSREQIEELRKLAKSSSKKSLPSEFEPFNLRSHKPEYSNKFGKLFEITPEKKYPQLQDLDILVSCVEINKGALMLPHYNSRAIVVLLVNEGKGNLELLGLKNEQQEREDRKERNNEVQRYEARLSPGDVVIIPAGHPVAISASSNLNLLGFGTNAENNQRNFLSGSDDN

SEQ ID NO: 232 - protein, 541 aa, LATHYRUS ANNUUS
MATTIKSRFPLLLLLGIIFLASVCVTWANYDEGSEPRVPGQRERGRQEGEKEEKRHGEWRPSYEEEYDEGLEPKVPGKRERGRQEGEKEEKRHEEWRPSYEKEEDEEEKQKYNYQREKKEHKEVQPGRERWERKQDEKQVEEDEEPGEEQWRGSKRHEDPEERARLRHREEKTKSYVEDNEETSSKEGRNPFLFKSNKFLTLFENENGHIRRLQRFDERSDIFENLQNYRLVEYRAKPHTMFLPQHIDADLILVVLNGKAILTVLSPNDRNSYNLERGDTVKLPAGTTSYLVNQDDEEDLRVVDLAIPVNRPGKFEAFGLSANKNQYLRGFSKNILEASLNTKYETIEKVLLEERRDQKGRQQGQETNAIVKVSREQIEELRKLAKSSSKKSLLSESEPLNLRSQNPKYSNKFGKFFEITPQKKYPQLQDLDVSISCVEINKGALLLPHYNSRSIGILLVNEGKGNLELVGFKNEQQRQRENEETNKKLQRYEARLSSGDVVVIPEGHPVAISASSNLNLLGFGINAANNQRNFLTGSDDN

SEQ ID NO: 233 - protein, 589 aa, Vicia villosa
MATTIKSRFPVLLLLGIIFLTSVCVTYANYDEGREPSVPGQRERGRQEGEKEEKRHGEWRPSEEDEEEKYKYEEGRVPGQRERGRQEGEKEEKRHGKWRPSEEEDEEEKYRYEEGSEPRGPGQRETGRQEGEKEKQRPEREPSYEKEEDEEEKQKYQYHREKKEQREVRPGRERFERHEDEEQWRGIQRHEDPEERARERYRAEIAKRQVEEEREERDIPHEREQRNPFLFKSNKFQTLFQNENGYIRRLQRFDKRSDLFENLQNYRLVEYRAKPHTIFLPQHIDADLIIVVLSGRAILTVLSPDDRNSYNLERGDTIKLPAGTTSYLVNQDDEEDLRVVDLAIPVNRPGKVESFLLSGNKNQYLRGFSKNILEASFNTNYETIERVLLEEQDKESQQSIGQKRRSQRQETNALVKVSREQLEDLKRLAKSSSQEGLSSQFEPINLRSQNPKYSNKFGKVFEITPEKKYPQLQDLDLFVSSVDIKEGALMLPHYNSRAIVVLLVNEGRGNLELVGLKNEQQEQREKEDEQQERNNQVQRYEARLSPGDVVIIPAGHPVAVRASSDLNLLAFGINAENNQRNFLAGSDDN

SEQ ID NO: 234 - protein, 463 aa, Medicago truncatula
MAIKAPFQLLMLLGIFFLASVCVSSRDDRHDQENPFFFNANHFQTLFENENGHIRLLQRFDKRSKIFENLQNYRLLEYHSKPHTLFLPQHNDADFILAVLSGKAILTVLNPDNRNSFNLERGDTIKLPAGSIAYLANRDDNEDLRVLDLAIPVNRPGKFQSFSLSGSQNQQSFFSGFSKNILEAAFNANYEEIERVLIEEHEQEPQHRRGLRKDRRQQSQDSNVIVKVSREQIEELSRHAKSSSRRSGSSESAPFNLRSREPIYSNEFGNFFEITPEKNPQLKDLDILVNYAEIREGSLLLPHFNSRATVIVVVDEGKGEFELVGQRNENQQEQREEDEQQEEERSQQVQRYRARLSPGDVYVIPAGHPTVVSASSDLSLLGFGINAENNERNFLAGEEDNVISQIERPVKEVAFPGSAQDVESLLKNQRQSYFANAQPQQREREEGRSQRQRELISSILGVF

SEQ ID NO: 235 - protein, 499 aa, VICIA PEREGRINA
MATTFKSRFSLLLLLGIIFLAFVCVTCANYDEGSEPRVPGQRERGRQEGEKEEQSRERHPQREPSREKEEDEEEKQKYDEGTEPRVPGQRERGRQEGEKEEQRRERHPGQREPSQEEDEEREESDRRQEGSSKSEEQRNPFLFKSNKFLTLFQNGNGHIRLLQRFDKRSDLFENLQNYRLLEYRAKPHTIFLPQHIDADLILVVLSGRAILTVLSPDDRNSYNLERGDTIKLPAGTTSYPLNQDDEEDLRVVDLAISVNRPGKVESFNLSGNKNQYLRGFSENILEASFNTKYETIEKVLLEEQDKESQQPRGQRLQRQETNALVKVSREQVEELKRLARTSSKKGVSSEFEPFNLRSHGPKYSNKFGKFFEITPEKKYPQLQDLDISVSSVEINEGALFLPHYNSRAIVVVLVDEGKGNLELVGFKNEQQEQREKEDEQEERNKQVQRYEAKLSPGDVVIIPAGHPVAVSASSNLNLLGFGINAENNQRNFLTGSDDN

SEQ ID NO: 236 - protein, 515 aa, VICIA LUTEA
MATTIKLRFPLLLLLGVILLASVCVTCANYDEGSEPRVPGRPEGEKEEKHRGKLRPSYEKEEDEGEKQRYHYEKKEQKEAQPRREKKEQKEEEKQVEEESRESQRYEDPGERARERYRAEIIKRQVEKEREERDRRHQREGEEEEGSSKSRNPFLFKSNNFLTLFENENGHIRLLQRFDKRSDLFENLQNYRLVEYRAKPHTIFLPQHIDADLILVVLSGKAILTVLSPNNRNSYNLKRGDTIKLPAGTTSYLLNSDDEEDLRMVDLAISVNRPGKVESFNLSGNKNQYLRGFSKNILEASFNTKYETIEKVLLEEQDKESQQSIGQKRISQRQETNALVKVSREQIEEPKRLARSSSRKGVSSEFEPINLRSQRPKYSNKFGKFYEISPEKKYPQLQDLDVSVSSVEINEGALLLPHYNSRAIVTVLVNEGKGNLELIGFQNEQQGQREKEDEQQHERNKQVQRYDARLSSGDVVIIPAGHPVAVSASSNLDLLGFGINAENSQRNFLTGSDDN

SEQ ID NO: 237 - protein, 494 aa, Oryza brachyantha
MATTTFSRFSIYFCVLLLCHGSMAQLFSPTLNPWHSSRRGGSRDCRFDRLQAFEPLRRVRSEAGVTEYFDERNEQFQCTGTFVIRRVIEPQGLLVPRYTNTPGVVYIMQGRGSMGLTFPGCPATYQQQFQQFLPEGQSQSQKFRDEHQKIHQFRQGDIVALPAGVAHWFYNEGDTPVVALYVFDINNSANQLEPRQKDFLLAGNNNREQQVYGRSIEKHSGQNIFSGFNHELLSEALGISTLAAKRLQGQNDHRGEIIRVRNGLQLLKPTFTQQQEQAQSQYQVQYSEKQQESTRCNGLDENFCTINARLNIENPSRADTYNPRAGRITHLNNQKFPILNLVQMSATRVNLYQNAILSPYWNVNAHSLVYMVQGHARVQVVSNLGKTVFNSVLRPGQLLIIPQHYVVLKKAEREGCQYIAFKTNANSIVSQLAGKNSILRAMPVDVVANAYRISREQARDLKNNRGEELGAFTPKFEQQSYPGLSNESESEASE

SEQ ID NO: 238 - protein, 515 aa, Avena sativa
MATTSFPSVLFYSCIFLLYNGSMAQLFGQSFTPWQSSRQGGLKGCKFDRLQAFEPLRQVRSQAGVTEYFDEQNEQFRCTGVFVIRRVIEPQGLLLPQYHNAPGLVYILQGRGYTGLTFPGCPATFQQQFQPFDQAQDQSQSHLKDEHQRVHRFKQGDVIALPAGIVHWGYNDGDAPVVAIYVFDVNNNANQLEPRQKEFLLAGNNKEDQQFGQNIFSGFNIQLLSEALGISQQAAQRIQSQKEQRGEIIRVTQALQFLKPTMSQQELVEHQAYQPIQSQEGQSTQYQVGQSTQYQEGQSTQYQAGQSQDRSFNGLEENFCSLEARQNIGNPKRADTHNPRAGRITRLHGQNFPILNLVQMSATRVNLYQNAILSPFWNINAHSVVYMIQGHAQVQVVNNNGQTVFNDRLRQGQLLIVPQHYVVLKKAEREGCQYISFKTNPNSMVSHIAGKSSILRALPVDVLANAYRISRQEARNLKNNRGQESGVFTPKFTQTSFQPYPEGEDESSLTNKASE

SEQ ID NO: 239 - protein, 493 aa, Brachypodium distachyon
MAHTSFSSVLSYFCIFLLFHGSMAQVPGQGSTWQSPRQGGSRECSFDRLQTIEPLTQVRSQAGLTEYFDEQNEQFRCAGVSVIRRVIEPRGLLLPRYHNTPGLVYILEGSGFVGLAFPGCPETFLEQFQQSRQTQSTLGQSQCQSQSQKLGDVHQRVHQFTQGDVVALPAGVAHWFYNGGDAPVVAVYVFDVNNNANQLEPRQKEFLLAGNYNGVLQSGRNILNGLNAQLLSQAFGINEQTSRIIQNQNDGRGEIVRVEYGLQFLTPVVTQQQQKQPFLPIEPQEGQSSRNGLEENFCSLEPRQNIEDPNRADTYNPRAGSIARLNGQNFPILNLVQMSATRVNLQKNAIVSPFWNINAHSVVYVIQGQASVQVVNNQGRNVFNGLLRRGQLLIIPQNYVVLKKAESEGYQYIAFKTNANSMVSHIAGKNSILRALPVDVIANAYRISRQEAQNLKNNRGEEIGVLTPNFPQSSCQSYPIGDVDSSSTPKAQE

SEQ ID NO: 240 - protein, 489 aa, Oryza sativa
MAQFSFGGSPLQSPRGFRGDQDSRHQCRFEHLTALEATHQQRSEAGFTEYYNIEARNEFRCAGVSVRRLVVESKGLVLPMYANAHKLVYIVQGRGVFGMALPGCPETFQSVRSPFEQEVATAGEAQSSIQKMRDEHQQLHQFHQGDVIAVPAGVAHWLYNNGDSPVVAFTVIDTSNNANQLDPKRREFFLAGKPRSSWQQQSYSYQTEQLSRNQNIFAGFSPDLLSEALSVSKQTVLRLQGLSDPRGAIIRVENGLQALQPSLQVEPVKEEQTQAYLPTKQLQPTWLRSGGACGQQNVLDEIMCAFKLRKNIDNPQSSDIFNPHGGRITRANSQNFPILNIIQMSATRIVLQNNALLTPHWTVNAHTVMYVTAGQGHIQVVDHRGRSVFDGELHQQQILLIPQNFAVVVKARREGFAWVSFKTNHNAVDSQIAGKASILRALPVDVVANAYRLSREDSRHVKFNRGDEMAVFAPRRGPQQYAEWQINEK

SEQ ID NO: 241 - protein, 489 aa, Oryza sativa
MAQFSFGGSPLQSPRGFRGDQDSRHQCRFEHLTALEATHQQRSEAGFTEYYNIEARNEFRCAGVSVRRLVVESKGLVLPMYANAHKLVYIVQGRGVFGMALPGCPETFQSVRSPFEQEVATAGEAQSSMQKMRDEHQQLHQFHQGDVIAVPAGVAHWLYNNGDSPVVAFTVIDTSNNANQLDPKRREFFLAGKPRSSWQQQSYSYQTEQLSRNQNIFAGFNPDLLSEALSVSKQTVLRLQGLSDPRGAIIRVENGLQALQPSLQVEPVKEEQTQAYLPTKQLQPTWSRSGGACGQQNGLDEIMCAFKLRKNIDNPQSSDIFNPHGGRITRANSQNFPILNIIQMSATRIVLQNNALLTPHWTVNAHTVMYVTAGQGRIQVVDHRGRSVFDGELHQQQILLIPQNFAVVVKARREGFAWVSFKTNHNAVDSQIAGKASILRALPVDVVANAYRLSREDSRRVKFNRGDEMAVFAPRRGPQQYAEWQINEK

SEQ ID NO: 242 - protein, 504 aa, Oryza brachyantha
MVDMSIVVPVCLTIFLLSQVCIAQVSFDGSPLYSSRGFRGGSASQQQCRFEHLAALEVTHQEKSEAGSIEYYNTEARDEFRCARVSARRLVIESRGLVLPVYANAHKLLYIVQGRGVFGMALPGCPETFQSVRSAFEMATGDAESSTRKLRDEHQKIHQFRQGDVIAVPPGVAHWLYNNGDSPVVAFSVIDFGNNANQLDPKPREFFLAGKPWGWQQVQYSYQSEQQSKHQNIFAGFNPDLLAEALSVSRQTAMRLQELNDQRGAIIRVEQGLQLALDPSFQAEQEQEEQPQEYLSSQQQQPTWSQRSGACVQNNGLDEIMCAFKVSKNINSAQSTDIFNPRGGRITRANSQNFPVLNIIQMSATRTVLQNNALLTPHWTVNAHTVMYVTAGQGRIQVVDHRGRTVFDGELRQQQILLIPQNFAVAVKARHEGFSWVSFKTSHNAIDSQIAGKGSILRALPVDVLAKAYMLSREESRTLKYNRADETLVFAPRPEIQLYAESEK

SEQ ID NO: 243 - protein, 543 aa, Pisum fulvum
MATTTKSRFPLLLLLGIIFLASVVCVTYANYDEGSEPRVPGRRERGRQEGEKEEKRHGEWRPSYEKEEDEEEGQRERGRQEGEKEEKRHGEWGPSYEKQEDEEEKQKYRYQREKEDEEEKQKYRYQREKKEQKEVQPGRERWEREEDEEHVDEEWRGSQRHEDPEERARLRYREERTKRDRRHQREGEEEERSSESQERRNPFLFKSNKFQTLFENENGHIRLLQRFDKRSDLFENLQNYRLVEYRAKPHTIFLPQHIDADLILVVLSGKAILTVLSPNARNSYNLERGDTIKLPAGTTSYLVNQDDEEDLRLVDLVIPVNGPGKFEAFDLSKNKNQYLRGFSKNILEASYNTKYETIEKVLLEEQEKTDAIVKVSREQIEELRKHAKSSSKKIFPSEFEPINLRNHKPEYSNKFGKLFEITPEKKYPQLQDLDIFVSCVEINEGALMLPHYNSRAIVVLLVNEGKGNLELLGLENEQQEREDRKERNNEVQRYEARLSPGDVVIIPAGHPVAITASSNLNLLAFGINAENNQRNFLSGSDDN

SEQ ID NO: 244 - protein, 526 aa, PISUM ABYSSINICUM; identical to SEQ ID NO: 231

SEQ ID NO: 245 - protein, 589 aa, Vicia villosa; identical to SEQ ID NO: 233

SEQ ID NO: 246 - protein, 576 aa, Lathyrus hirsutus
MAIIIKSRFPLLLLLGIIFLASVCATWANYDEGSEPRVPGQRERGRQEGEKAEKSHEKWRPSYEEEYDEGSEPRVPGKRERGRQEGEKEEKRHGEWRPSHEEEYDEGSEPRVPTHGERGRQEGEKEEKRHEEWRPSYEKEEDEEEKEKYKYQREKKEQKEVQPGREKWERKQDEKHVEEDEDQEEEQWRGSKRREDPEERARLRYREERTKSNVEEETEERRNPFLFKSNKFLTLFENENGHIRRLQRFDERSDIFENLQNYRLVEYKAKPHTMFLPQHIDADLIIVVLNGKAILTVLSPNDRNSYNLERGDTIKLPAGTTSYLVNQDDEEDLRVVDLAIPVNRPGKFEAFGLSANKNQYLRGFSKNILEAFLNTKYETIEKVLLEEQERRDRKGRQQGQETNAIVKVSREQIEELRKLAKSSSKKSLLSESEPINLRSQNPKYSNKFGKLFEITPEKKYPQLQDLDVSISCVEINEGAPLLPHYNSRAIVLLLVNEGKGNLELVGFKNEQQRQRENEERNKKVQRYEARLSPGDVVVIPAGHPVAISASLNLNLVGFGVNAENNQRNFLTGSDDN

SEQ ID NO: 247 - protein, 564 aa, Lathyrus cicera
MATIIKSRFPLLLLLGIIFLASVCVTLANYDEGSEPRVPAQRERGRQEGEKEEKRHGEWRPSHEKEYDEGSEPRVPGRRERGRQEGEKEEKRHGEWRPSYEKEYDEGSEPRVPGRRERGRQEGEKEEKRHGEWRPSYEKEYDEEEKQKYQYEREKEEQKEVQPGRERWERKEDEEKEEDQWRGSQRHEDPEERARLRYRKERTKKYVEEDTEETSSESQGRRNPFLFKSNKFLTLFENENGYIRRLQRFDERSDIFENLQNYRLVEYRAKPHTIFLPQHIDADLILVILNGKAILTVLSPNDRNSYNLERGDTIKLPAGTTSYLVNEDDEEDLRVVDLVIPVNRPGKFEAFDLNQYLGGFSKSVLEASLNTKYETIEKVLLEEQQKQGQETNAIVKVSREQIEELRKLAKSSSKKSLLSELEPVNLRSHSPKYSNKFGKFFEITPEKKYPQLQDLDVSISCVEINEGALLLPHYNSRAIVVVLVNEGKGNLELLGVQNEDEQQERKERNKEVQRYEARLSPGDVVIIPSGHPVAVSASSNLNLLGFGINAENNQRNFLSGSDDN

SEQ ID NO: 248 - protein, 527 aa, Lathyrus sativus
MATIIKSRFPLLLLLGIIFLASVCVTYANYDEGSEPRVPAQRERGRQEGEKEEKRHGEWRPSSEKEYDEGSEPRVPGRRERGRQEGEKEEKRHGEWRPSYEKEYDEEEKQKYQYEREKKEQKEVEPGRERWERKEDEEKEEDQWRGSQRHEDPEERARLRYRKERTKKYVEEDTEETSSESQGRRNPFLFKSNKFLTLFENENGYIRRLQRFDERSDLFENLQNYRLVEYRAKPHTIFLPQHIDADLILVILNGKAILTVLSPNDRNSYNLERGDTIKLPAGTTSYLVNEDDEEDLRVVDLVIPVNRPGKFEAFDLNQYLGGFSKSVLKASLNTKYETIEKVLLEEQQKQGQETNAIVKVSREQIEELRKLAKSSSKKSLLSELEPVNLRSHSPKYSNKFGKFFEITPEKKYPQLQDLDVSISCVEINEGALLLPHYNSRAIVVLLVNEGKGNLELLGVQDEDEQQERKKRNKEVQRYEARLSPSDVVIIPAGHPVAVSASSNLNLLGFGINAENNERNFLSGSDDN

SEQ ID NO: 249 - protein, 495 aa, Oryza brachyantha
MATTVFSRFSTYFCVLLLCHGSMAQLFNPSTNPWHNPRQGSSRECRFDRLQPFEPLRKVRSEAGVTEYFDEKNELFQCTGTFVIRRVIQPQGLLVPRYTNAPGLVYIIQGRGSIGLTFPGCPATYQQQFQQFLPQEQSQSQKFRDEHQKIHQFRQGDIVALPAGVAHWFYNDGDAPVVAVYVYDVKNSANQLEPRQREFLLGGNNMRAQQVYGSSAEQHSRQNIFSGFGVEILSEALGISTVTTKRLQSQNDQRGEIIHVKNGLQFLKPTLTQQQEQAQAQYQEVQYSEQQQTSSRWNGLDENFCTIKARMNIENTSRADTYNPRAGRTTSLNSQKFPILNLVQMSATRVNLYQNAILSTFWNVNAHSLVYTIQGRARVQVVSNFGKTVFDGELRPGQLLIIPQHYVVLKKAQREGFRYIAIKTNANAFVSQLVGKNSVFRSLPVDVIANVYRISREQARSLKNNRGEEHGAFAPRSQQQSYPGFSNQSESETSE

SEQ ID NO: 250 - protein, 494 aa, Oryza brachyantha; identical to SEQ ID NO: 237

SEQ ID NO: 251 - protein, 500 aa, Zizania latifolia
MNMATINGPTIFFTVCLFLLCHGSLAQLLGQSTSQWQSSHRGSSRQCRFDRLQAFEPVRSVRSQAGTTEFFDASNELFQCAGVSIVRRIIEPRGLLLPQYTNGATIMYIIQGRGITGQTFPGCPESYQQQFQQSMQAQLTGSQSQSQKFKDEHQKINRFRQGDVIALPAGVAHWCYNDGEVPVVAIYVIDINNAANQLDPRQRDFLLAGNMRSPQAYRREVENQSQNIFSGFSAELLSEALGISTGVARQLQCQNDQRGEIVRVEHGLSLLQPYASLQEQEQKQEQPRERYQVTQHQQSQYGGGCSNGLDETFCAMRIWQNIDNPNLADTYNPRAGRVTNLNSQKFPILNLIQMSAVKVNLYQNALLSPFWNINSHSVVYVTQGCARVQVVNNNGKTVFNGELRRGQLLIIPQHYVVVKKAQREGCAYIAFKTNPNSMVSHIVGKSSIFRALPTDVLANAYRISREDAQRLKHNRGDELGAFTPLQYKSYQDVSSVAASS

SEQ ID NO: 252 - protein, 454 aa, Oryza sativa
CRFDRLQAFEPIRSVRSQAGTTEFFDVSNEQFQCTGVSAVRRVIEPRGLLLPHYTNGASLVYIIQGRGITGPTFPGCPESYQQQFQQSGQAQLTESQSQSHKFKDEHQKIHRFRQGDVIALPAGVAHWCYNDGEVPVVAIYVTDLNNGANQLDPRQRDFLLAGNKRNPQAYRREVEERSQNIFSGFSTELLSEALGVSSQVARQLQCQNDQRGEIVRVEHGLSLLQPYASLQEQEQGQVQSRERYQEGQYQQSQYGSGCSNGLDETFCTMKVRQNIDNPNRADTYNPRAGRVTNLNTQNFPILNLVQMSAVKVNLYQNALLSPFWNINAHSVVYITQGRARVQVVNNNGKTVFNGELRRGQLLIIPQHYAVVKKAQREGCAYIAFKTNPNSMVSHIAGKSSIFRALPNDVLANAYRISREEAQRLKHNRGDEFGAFTPIQYKSYQDVYNAAESS

SEQ ID NO: 253 - protein, 500 aa, Zizania latifolia; identical to SEQ ID NO: 251

SEQ ID NO: 254 - protein, 527 aa, Avena sativa
MATTSFPSMLFYFCIFLLFHGSMAQLFGQSSTPWQSSRQGGLRGCRFDRLQAFEPLRQVRSQAGITEYFDEQNEQFRCTGVSVIRRVIEPQGLVLPQYHNAPALVYILQGRGFTGLTFPGCPATFQQQFQPFDQSQFAQGQRQSQTIKDEHQRVQRFKQGDVVALPAGIVHWCYNDGDAPIVAIYVFDVNNNANQLEPRQKEFLLAGNNKREQQSGNNIFSGLSVQLLSEALGISQQAAQRIQSQNDQRGEIIRVSQGLQFLKPIVSQQVPGEQQVYQPIQTQEGQATQYQVGQSTQYQVGKSTPYQGGQSSQYQAGQSWDQSFNGLEENFCSLEARKNIENPQHADTYNPRAGRITRLNSKNFPILNIVQMSATRVNLYQNAILSPFWNINAHSVIYMIQGHARVQVVNNNGQTVFNDILRRGQLLIVPQHFVVLKKAEREGCQYISFKTNPNSMVSHIAGKSSILRALPIDVLANAYRISRQEARNLKNNRGEEFGAFTPKLTQKGFQSYQDIEEGSSSPVRASE

SEQ ID NO: 255 - protein, 498 aa, Oryza sativa
MASTNRPIVFFTVCLFLLCDGSLAQQLLGQSTSQWQSSRRGSPRGCRFDRLQAFEPIRSVRSQAGTTEFFDVSNELFQCTGVSVVRRVIEPRGLLLPHYTNGASLVYIIQGRGITGPTFPGCPETYQQQFQQSGQAGLTESQSQSHKFKDEHQKIHRFRQGDVIALPAGVAHWCYNDCEVPVVAIYVTDINNGANQLDPRQRDFLLAGNKRNPQAYRREVEEWSQNIFSGFSTELLSEAFGISNQVARQLQCQNDQKGEIVRVERGLSLLQPYASLQEQEQGQMQSREHYQEGGYQQSQYGSGCPNGLDETFCVNKVRQNIDNPNRADTYNPRAGRVTNLSQNFPILNLVQMSAVKVNLYQNTDTWISMGQEENALLSPFWNINAHSIVYITQGRAQVQVLRRGQLLIVPQHYVVVKKAQREGCAYIAFKTNPNSMVSHIAGKSSIFRALPTDVLANAYRISREEAQRLKHNRGDEFGAFTPLQYKSYQDVYNVAESS

SEQ ID NO: 256 - protein, 491 aa, Oryza brachyantha
MKSSIVFSTICLVLLCHGSLAQLLSQSTSQWQSSRRGSPRQCRFDQLQAFEPIRTVRSQAGVTEFYDVSNELFQCTGVSVVRRVIEPRGLLLPHYSNGATLVYIIQGRGITGPTFPGCPETYQQQFQQSGEAQPFEGQSHKFRDEHQKIHRFRQGDVVALPAGVAHWCYNDGEVPIVAIYVTDIYNSANQLDPRHRDFFLAGNNKVAQQLYRSEARENSKNIFGGFSVELLSEALGISRGVARQLQCQNDQRGEIVRVEHGLALLQPYASVQEQQQEQVQSRDYEQTQYQQKQPQGSCSNGLDETFCTMRLRQNIDNPNLADTYNPKAGRITYLNGQKFPILNLVQMSAVKVNLYQNAVLSPFWNINAHSVVYITQGRARVQVVNNNGKTVFDGELRQGQLLIIPQHHVVLKKAQREGCSYIALKTNPNSIVSHIAGKNSIFRALPGDVVTNAYRISREEAKRIKHNRGDESGVFAPSHAYRSYQDMSVAA

SEQ ID NO: 257 - protein, 492 aa, Brachypodium distachyon
MAHTSFSSFLSYFCLFLLFHGSMAQVLGQVSTWQSSRQGGSRDCSFDRLQAIEPVTQVRSQAGLTEYFDEQNEQFRCAGVFVIRRVIEPRGLLLPRYHNTPGLVYILQGNGFVGLTFPGCPETFREQFQQFRQTQSTLGQSQCQSQKLGDVHQRVHQFTQGDVVALPTGVAHWIYNGGDAPVVIVYVFDVNNNANQLEPRQKEFLLGGNYNGVLQYGQNIFSGFNAQLLSQAFGINEQTSQRIQNQNDGRGDIIRVDNGLQFLKPVVTQQQPEQPFMPIQHQTGQSSRNGLEENFCSLEPRQNIEDPNRADTYNPRAGSITRLNGQNFPILNLVQMSATRVNLQKNAILSPFWNINAHSVVYVIQGHALVQVVNNQGHNVFNGLLHRGQLLIIPQNYVVLKKAESEGYQYIAFKTNANSMVSHIAGKNSILRALPVDVIANAYRISRQEAQNLKNNRGEETGVLTPNFSQSTCQSYQTEDVQSLRPMSHWSE

SEQ ID NO: 258 - protein, 166 aa, Oryza sativa
  SITE 120: note = misc_feature - Xaa can be any naturally occurring amino acid
MASNKVVFSALLLIIVSVLAATATMADHHKDQVVYSLGERCQPGMGYPMYSLPRCRAVVKRQCVGHGAPGGAVDEQLRQDCCRQLAAVDDSWCRCSALNHMVGGIYRELGATDVGHPMAXVFPGCRRGDLERAAASLPAFCNVDIPNGTGGVCYWLGYPRTPRTGH

SEQ ID NO: 259 - protein, 165 aa, Oryza sativa
MASNKVVFSALLLIIVSVLRRDGTMADHHKDQVVYSLGERCQPGMGYPMYSLPRCRAVVKRQCVGHGAPGAVDEQLRQDCCRQLAAVDDSWCRCSALNHMVGGIYRELGATDVGHPMAEVFPGCRRGDLERAAASLPAFCNVDIPNGTGGVCYWLGYPRTPRTGH

SEQ ID NO: 260 - protein, 157 aa, Oryza sativa
MASNKVVISALLVVVVSVLAATTTMADHHQEQVVYTPGQLCQPGIGYPTYPLPRCRAFVKRQCVAPGTVDEQVRRGCCRQLAAIDSSWCRCDALNHMLRIIYRESGAADAGHPMAEVFRGCRRGDIERAAASLPAFCNVDIPNGVGGVCYWLPGTGY

SEQ ID NO: 261 - protein, 495 aa, Oryza sativa
MTISVFSRFSIYFCVLLLCNGSMAQLFDPATNQWQTHRQGSFRECRFERLQAFEPLQNVRSEAGVTEYFDETNELFQCTGTFVIRRVIQPQGLLIPRYANTPGMVYIIQGRGSMGLTFPGCPATYQQQSQQFLFQGESQSQKFIDEHQKIHQFRQGDIVVLPTGVAHWFYNDGDTPVVALYVYDINNSANQLEPRHREFLLAGKNNRVQQVYGRSIQQHSGQNIFNGFSVEPLSEALNINTVTTKRLQSQNDQRGEIIHVKNGLQLLKPTLTQRQEQEQAQYQEVQYSEKPQTSSRWNGLEENLCTIKTRLNIENPSRADSYDPRAGRITSLDSQKFPILNIIQMSATRVNLYQNAILTPFWNVNAHSLMYVIRGRARVQVVSNFGKTVFDGVLRPEQLLIIPQNYVVLKKAQHEGCQYIAINTNANAFVSHLAGVDSVFHALPVDVIANAYCISREEARRLKNNRGDEYGPFPPRLQQQIYPEFSNESKGETSE

SEQ ID NO: 262 - protein, 480 aa, Oryza sativa
LLCHGSMAQIFSLGINPWQNPRQGGSRECRFDRLQAFEPLRKVRHEAGVTEYFDEKNEQFQCTGTLVIRRIIEPQGLLLPRYSNTPGLVYIIQGTGVLGLTFPGCPATYQKQFRHFGLEGGSQRQGKKLRDENQKIHQFRQGDVVALPSGIPHWFYNEGDTPVVALFVFDVNNNANQLEPRQKEFLLAGNNIEQQVSNPSINKHSGQNIFNGFNTKLLSEALGVNIEVTRRLQSQNDRRGDIIRVKNGLRLIKPTITQQQEQTQDQYQQIQYHREQRSTSKYNGLDENFCAIRARLNIENPNHADTYNPRAGRITNLNSQKFSILNLVQMSATRVNLYQNAILSPFWNINAHSLVYTIQGRARVQVVSNHGKAVFNGVLRPGQLLIIPQNYVVMKKAELEGFQFIAFKTNPNAMVNHIAGKNSVLRAMPVDVIANAYRISRQEARSLKNNRGEEIGAFTPRYQQQKIHQEYSNPNESETQ

SEQ ID NO: 263 - protein, 518 aa, Avena sativa
MATTRFPSLLFYSCIFLLCNGSMAQLFGQSFTPWQSSRQGGLRGCRFDRLQAFEPLRQVRSQAGITEYFDEQNEQFRCAGVSVIRRVIEPQGLLLPQYHNAPGLVYILQGRGFTGLTFPGCPATFQQQFQPFDQARFAQGQSKSQNLKDEHQRVHHIKQGDVVALPAGIVHWCYNDGDAPIVAVYVFDVNNNANQLEPRQKEFLLAGNNKREQQFGQNIFSGFSVQLLSEALGISQQAAQKIQSQNDQRGEIIRVSQGLQFLKPFVSQQGPVEHQAYQPIQSQQEQSTQYQVGQSPQYQEGQSTQYQSGQSWDQSFNGLEENFCSLEARQNIENPKRADTYNPRAGRITHLNSKNFPTLNLVQMSATRVNLYQNAILSPYWNINAHSVMHMIQGRARVQVVNNHGQTVFNDILRRGQLLIIPQHYVVLKKAEREGCQYISFKTTPNSMVSYIAGKTSILRALPVDVLANAYRISRQESQNLKNNRGEEFGAFTPKFAQTGSQSYQDEGESSSTEKASE

SEQ ID NO: 264 - protein, 605 aa, Oryza rufipogon
MSALTTSQLATSATGFGIADRSAPSSLLRHGFQGLKPRSPAGGDATSLSVTTSARATPKQQRSVQRGSRRFPSVVVYATGAGMNVVFVGAEMAPWSKTGGLGDVLGGLPPAMAANGHRVMVISPRYDQYKDAWDTSVVAEIKVADRYERVRFFHCYKRGVDRVFVDHPSFLEKVWGKTGEKIYGPDTGVDYKDNQMRFSLLCQAPRILNLNNNPYFKGTYGEDVVFVCNDWHTGPLASYLKNNYQPNGIYRNAKVAFCIHNISYQGRFAFEDYPELNLSERFRSSFDFIDGYDTPVEGRKINWMKAGILEADRVLTVSPYYAEELISGIARGCELDNIMRLTGITGIVNGMDVSEWDPSKDKYITAKYDATTAIEAKALNKEALQAEAGLPVDRKIPLIAFIGRLEEQKGPDVMAAAIPELMQEDVQIVLLGTGKKKFEKLLKSMEEKYPGKVRAVVKFNAPLAHLIMAGADVLAVPSRFEPCGLIQLQGMRYGTPCACASTGGLVDTVIEGKTGFHMGRLSVDCKVVEPSDVKKVAATLKRAIKVVGTPAYEEMVRNCMNQDLSWKGPAKNWENVLLGLGVAGSAPGIEGDEIAPLAKENVAAP

SEQ ID NO: 265 - protein, 609 aa, ORYZA OFFICINALIS
MSALTTSQLATSATGFGIADRSAPSSLLRHGFQGLKPRSPAGGDASSLSVTTSARATPKQQRSVQRGSRRFPSVVVYATGAGMNVVFVGAEMAPWSKTGGLGDVLGGLPPAMAANGHRVMVISPRHDQYKDAWDTSVVAEIKVADRYERVRFFHCYKRGVDRVFIDHPSFLEKVWGKTGEKIYGPDTGVDYKDNQMRFSLLCQAALEAPRILNLNNNPYFKGTYGEDVVFVCNDWHTGPLPSYLKNNYQPNGIYRNAKVAFCIHNISYQGRFAFEDYPELNLSERFRSSFDFIDGYDTPVEGRKINWMKAGILESDRVLTVSPYYAEELISGIARGCELDNIMRLTGITGIVNGMDVSEWDPSKDKYIAAKYDATTAIEAKALNKEALQAEAGLPVDRKIPLIAFIGRLEEQKGPDVMAAAIPELMQENVQIVLLGTGKKKFEKLLKSMEEKYPGKVRAVVKFNAPLAHLIMAGADVLAVPSRFEPCGLIQLQGMRYGTPCACASTGGLVDTVIEGKTGFHMGRLSVDCKVVEPSDVQKVATTLKRAIKIVGTPAYNEMVRNCMNQDLSWKGPAKNWENVLLGLGVAGSAPGVEGEEIAPLAKENVAAP

SEQ ID NO: 266 - protein, 603 aa, Hordeum vulgare
MAALATSQLATSGTVLGVTDRFRRPGFQGLRPRNPADAALGMRTIGASAAPKQSRKAHRGSRRCLSVVVRATGSGMNLVFVGAEMAPWSKTGGLGDVLGGLPPAMAANGHRVMVVSPRYDQYKDAWDTSVISEIKVADEYERVRFFHCYKRGVDRVFIDHPWFLEKVRGKTKEKIYGPDAGTDYEDNQQRFSLLCQAALEAPRILNLNNNPYFSGPYGEDVVFVCNDWHTGLLACYLKSNYQSNGIYRTAKVAFCIHNISYQGRFSFDDFAQLNLPDRFKSSFDFIDGYDKPVEGRKINWMKAGILQADKVLTVSPYYAEELISDEARGCELDNIMRLTGITGIVNGMDVSEWDPTKDKFLAVNYDITTALEAKALNKEALQAEVGLPVDRKVPLVAFIGRLEEQKGPDVMIAAIPEILKEEDVQIILLGTGKKKFEKLLKSMEEKFPGKVRAVVRFNAPLAHQMMAGADLLAVTSRFEPCGLIQLQGMRYGTPCVCASTGGLVDTIVEGKTGFHMGRLSVDCNVVEPADVKKVATTLKRAVKVVGTPAYQEMVKNCMIQDLSWKGPAKNWEDVLLELGVEGSEPGIVGEEIAPLAMENVAAP

SEQ ID NO: 267 - protein, 336 aa, Staphylococcus aureus
MKGKFLKVSSLFVATLTTATLVSSPAANALSSKAMDNHPQQSQSSKQQTPKIQKGGNLKPLEQREHANVILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDEPNNPDNPNNPDNPDNGDNNNSDNPDAA

SEQ ID NO: 268 - protein, 14 aa, synthetic construct
  REGION 1..14: note = Synthetic peptide
RGPQQYAEWQINER

SEQ ID NO: 269 - protein, 14 aa, synthetic construct
  REGION 1..14: note = Synthetic peptide
RGPQQYAEWQINDK

SEQ ID NO: 270 - protein, 14 aa, synthetic construct
  REGION 1..14: note = Synthetic peptide
RGPQQFAEWQINEK

SEQ ID NO: 271 - protein, 14 aa, synthetic construct
  REGION 1..14: note = Synthetic peptide
KGPQQYAEWQINEK

SEQ ID NO: 272 - protein, 14 aa, synthetic construct
  REGION 1..14: note = Synthetic peptide
RGPEQYAEWQINEK

SEQ ID NO: 273 - protein, 14 aa, synthetic construct
  REGION 1..14: note = Synthetic peptide
RGPQEYAEWQINEK

SEQ ID NO: 274 - protein, 14 aa, synthetic construct
  REGION 1..14: note = Synthetic peptide
RGPQQYADWQINEK

SEQ ID NO: 275 - protein, 14 aa, synthetic construct
  REGION 1..14: note = Synthetic peptide
RGPQQYAEYQINEK

SEQ ID NO: 276 - protein, 14 aa, synthetic construct
  REGION 1..14: note = Synthetic peptide
KGPEQYAEWQINEK

SEQ ID NO: 277 - protein, 14 aa, synthetic construct
  REGION 1..14: note = Synthetic peptide
KGPQEYAEWQINEK

SEQ ID NO: 278 - protein, 14 aa, synthetic construct
  REGION 1..14: note = Synthetic peptide
KGPQQFAEWQINEK

SEQ ID NO: 279 - protein, 14 aa, synthetic construct
  REGION 1..14: note = Synthetic peptide
RGPEQFAEWQINEK

SEQ ID NO: 280 - protein, 14 aa, synthetic construct
  REGION 1..14: note = Synthetic peptide
KGPQQYAEWQINER

SEQ ID NO: 281 - protein, 14 aa, synthetic construct
  REGION 1..14: note = Synthetic peptide
RGPQQYAEWQINDR

SEQ ID NO: 282 - protein, 14 aa, synthetic construct
  REGION 1..14: note = Synthetic peptide
RGPQQYADWQINDK

SEQ ID NO: 283 - protein, 14 aa, synthetic construct
  REGION 1..14: note = Synthetic peptide
RGPQQFAEWQINER

SEQ ID NO: 284 - protein, 14 aa, synthetic construct
  REGION 1..14: note = Synthetic peptide
RGPQQYAEWQVNEK

SEQ ID NO: 285 - protein, 14 aa, synthetic construct
  REGION 1..14: note = Synthetic peptide
RGPQQFAEWQINEK

SEQ ID NO: 286 - protein, 14 aa, synthetic construct
  REGION 1..14: note = Synthetic peptide
KGPQQFAEWQINER

SEQ ID NO: 287 - protein, 14 aa, synthetic construct
  REGION 1..14: note = Synthetic peptide
KGPQQFAEWQVNEK

SEQ ID NO: 288 - protein, 14 aa, synthetic construct
  REGION 1..14: note = Synthetic peptide
RGPQQFAEWQVNDK

SEQ ID NO: 289 - protein, 14 aa, synthetic construct
  REGION 1..14: note = Synthetic peptide
RGPQQYADWQINDR

SEQ ID NO: 290 - protein, 14 aa, synthetic construct
  REGION 1..14: note = Synthetic peptide
KGPQQYADWQINDK

SEQ ID NO: 291 - protein, 14 aa, synthetic construct
  REGION 1..14: note = Synthetic peptide
RGPQQFADYQINEK

SEQ ID NO: 292 - protein, 14 aa, synthetic construct
  REGION 1..14: note = Synthetic peptide
RGPQQYARWQINEK

SEQ ID NO: 293 - protein, 14 aa, synthetic construct
  REGION 1..14: note = Synthetic peptide
RGPQQYAEWQINEE

SEQ ID NO: 294 - protein, 14 aa, synthetic construct
  REGION 1..14: note = Synthetic peptide
HGPQQYAEWQINEK

SEQ ID NO: 295 - protein, 14 aa, synthetic construct
  REGION 1..14: note = Synthetic peptide
RGPYQYAEWQINEK

SEQ ID NO: 296 - protein, 14 aa, synthetic construct
  REGION 1..14: note = Synthetic peptide
RGPQQYMEWQINEK

SEQ ID NO: 297 - protein, 14 aa, synthetic construct
  REGION 1..14: note = Synthetic peptide
RGPQQYAEWCINEK

SEQ ID NO: 298 - protein, 14 aa, synthetic construct
  REGION 1..14: note = Synthetic peptide
RGPQPYAEWQINEK

SEQ ID NO: 299 - protein, 14 aa, synthetic construct
  REGION 1..14: note = Synthetic peptide
RGGQQYAEWQINED

SEQ ID NO: 300 - protein, 14 aa, synthetic construct
  REGION 1..14: note = Synthetic peptide
RGPQQYARWKINEK

SEQ ID NO: 301 - protein, 14 aa, synthetic construct
  REGION 1..14: note = Synthetic peptide
RGGQQYAETQINEK

SEQ ID NO: 302 - protein, 14 aa, synthetic construct
  REGION 1..14: note = Synthetic peptide
RGPLQYAEWQNNEK

SEQ ID NO: 303 - protein, 14 aa, synthetic construct
  REGION 1..14: note = Synthetic peptide
EGPQQYAEWQINED

SEQ ID NO: 304 - protein, 14 aa, synthetic construct
  REGION 1..14: note = Synthetic peptide
RGPQQYAEWQINLL

SEQ ID NO: 305 - protein, 14 aa, synthetic construct
  REGION 1..14: note = Synthetic peptide
RGPQQGGEWQINEK

SEQ ID NO: 306 - protein, 14 aa, synthetic construct
  REGION 1..14: note = Synthetic peptide
RGPQQYAEWQIGGG

SEQ ID NO: 307 - protein, 14 aa, synthetic construct
  REGION 1..14: note = Synthetic peptide
RGPQQKYEWQINEK

SEQ ID NO: 308 - protein, 14 aa, synthetic construct
  REGION 1..14: note = Synthetic peptide
RGPQAQYEWQINEK

SEQ ID NO: 309 - protein, 14 aa, synthetic construct
  REGION 1..14: note = Synthetic peptide
RPHQQYAEWQINEK

SEQ ID NO: 310 - protein, 14 aa, synthetic construct
  REGION 1..14: note = Synthetic peptide
RGPQHHHEWQINEK

SEQ ID NO: 311 - protein, 14 aa, synthetic construct
  REGION 1..14: note = Synthetic peptide
RGPPQYAPPQINEK

SEQ ID NO: 312 - protein, 14 aa, synthetic construct
  REGION 1..14: note = Synthetic peptide
RGPQCYYEWCINEK

SEQ ID NO: 313 - protein, 14 aa, synthetic construct
  REGION 1..14: note = Synthetic peptide
RGPTQYAEGQINEG

SEQ ID NO: 314 - protein, 15 aa, synthetic construct
  REGION 1..15: note = Synthetic peptide
RGPQQYAEWQINEKG

SEQ ID NO: 315 - protein, 15 aa, synthetic construct
  REGION 1..15: note = Synthetic peptide
RGPQQYAEWQINEKY

SEQ ID NO: 316 - protein, 16 aa, synthetic construct
  REGION 1..16: note = Synthetic peptide
RGPQQYAFTEWQINEK

SEQ ID NO: 317 - protein, 17 aa, synthetic construct
  REGION 1..17: note = Synthetic peptide
RGPQSQYAEWQINEKPM

SEQ ID NO: 318 - protein, 16 aa, synthetic construct
  REGION 1..16: note = Synthetic peptide
RGPQQYAEWQINEKKK

SEQ ID NO: 319 - protein, 17 aa, synthetic construct
  REGION 1..17: note = Synthetic peptide
RRRRGPQQYAEWQINEK

SEQ ID NO: 320 - protein, 13 aa, synthetic construct
  REGION 1..13: note = Synthetic peptide
RGPQQYAEWQINE

SEQ ID NO: 321 - protein, 12 aa, synthetic construct
  REGION 1..12: note = Synthetic peptide
RGPQQYAEWQIN

SEQ ID NO: 322 - protein, 11 aa, synthetic construct
  REGION 1..11: note = Synthetic peptide
RGPQQYAEWQI

SEQ ID NO: 323 - protein, 13 aa, synthetic construct
  REGION 1..13: note = Synthetic peptide
GPQQYAEWQINEK

SEQ ID NO: 324 - protein, 12 aa, synthetic construct
  REGION 1..12: note = Synthetic peptide
PQQYAEWQINEK

SEQ ID NO: 325 - protein, 11 aa, synthetic construct
  REGION 1..11: note = Synthetic peptide
QQYAEWQINEK

SEQ ID NO: 326 - protein, 8 aa, synthetic construct
  REGION 1..8: note = Synthetic peptide
QQYAEWQI

SEQ ID NO: 327 - protein, 11 aa, synthetic construct
  REGION 1..11: note = Synthetic peptide
PQQYAEWQINE

SEQ ID NO: 328 - protein, 10 aa, synthetic construct
  REGION 1..10: note = Synthetic peptide
PQQYAEWQIN

SEQ ID NO: 329 - protein, 7 aa, synthetic construct
  REGION 1..7: note = Synthetic peptide
RGPQQYA

SEQ ID NO: 330 - protein, 7 aa, synthetic construct
  REGION 1..7: note = Synthetic peptide
EWQINEK

SEQ ID NO: 331 - protein, 7 aa, synthetic construct
  REGION 1..7: note = Synthetic peptide
EWQINEK

SEQ ID NO: 332 - protein, 7 aa, synthetic construct
  REGION 1..7: note = Synthetic peptide
FLPQHTD

SEQ ID NO: 333 - protein, 13 aa, synthetic construct
  REGION 1..13: note = Synthetic peptide
GPQQYAEWQINEK

SEQ ID NO: 334 - protein, 12 aa, synthetic construct
  REGION 1..12: note = Synthetic peptide
PGQLQSFLLSGN

SEQ ID NO: 335 - protein, 22 aa, synthetic construct
  REGION 1..22: note = Synthetic peptide
PGQLQSFLLSGNQNQQNYLSGF

SEQ ID NO: 336 - protein, 8 aa, synthetic construct
  REGION 1..8: note = Synthetic peptide
PQQYAEWQ

SEQ ID NO: 337 - protein, 22 aa, synthetic construct
  REGION 1..22: note = Synthetic peptide
QLQSFLLSGNQNQQNYLSGFSK

SEQ ID NO: 338 - protein, 12 aa, synthetic construct
  REGION 1..12: note = Synthetic peptide
QNQQNYLSGFSK

SEQ ID NO: 339 - protein, 12 aa, synthetic construct
  REGION 1..12: note = Synthetic peptide
QSFLLSGNQNQQ

SEQ ID NO: 340 - protein, 9 aa, synthetic construct
  REGION 1..9: note = Synthetic peptide
QSFLLSGNQ

SEQ ID NO: 341 - protein, 7 aa, synthetic construct
  REGION 1..7: note = Synthetic peptide
RGPQQYA

SEQ ID NO: 342 - protein, 8 aa, synthetic construct
  REGION 1..8: note = Synthetic peptide
DALEPDNR

SEQ ID NO: 343 - protein, 17 aa, synthetic construct
  REGION 1..17: note = Synthetic peptide
SEEGYYGEQQQQPGMTR

SEQ ID NO: 344 - protein, 14 aa, synthetic construct
  REGION 1..14: note = Synthetic peptide
GYYGEQQQQPGMTR

SEQ ID NO: 345 - protein, 11 aa, synthetic construct
  REGION 1..11: note = Synthetic peptide
IDGYDTPVEGR

SEQ ID NO: 346 - protein, 9 aa, synthetic construct
  REGION 1..9: note = Synthetic peptide
NGVLRPGQL

SEQ ID NO: 347 - protein, 9 aa, synthetic construct
  REGION 1..9: note = Synthetic peptide
RHGEWGPSY

SEQ ID NO: 348 - protein, 4 aa, synthetic construct
  REGION 1..4: note = Synthetic peptide
FWMA

SEQ ID NO: 349 - protein, 12 aa, synthetic construct
  REGION 1..12: note = Synthetic peptide
TVFDGVLRPGQL

SEQ ID NO: 350 - protein, 16 aa, synthetic construct
  REGION 1..16: note = Synthetic peptide
RLQSQNDQRGEIIHVK

SEQ ID NO: 351 - protein, 16 aa, synthetic construct
  REGION 1..16: note = Synthetic peptide
HGPVEMPYTLLYPSSK

SEQ ID NO: 352 - protein, 9 aa, synthetic construct
  REGION 1..9: note = Synthetic peptide
LDALEPDNR

SEQ ID NO: 353 - protein, 186 aa, Oryza sativa
MASKVVFFAAALMAAMVAISGAQLSESEMRFRDRQCQREVQDSPLDACRQVLDRQLTGRERFQPMFRRPGALGLRMQCCQQLQDVSRECRCAAIRRMVRSYEESMPMPLEQGWSSSSSEYYGGEGSSSEQGYYGEGSSEEGYYGEQQQQPGMTRVRLTRARQYAAQLPSMCRVEPQQCSIFAAGQY

SEQ ID NO: 354 - protein, 517 aa, Pisum sativum
MAKLLALSLSFCFLLLGGCFALREQPQQNECQLERLDALEPDNRIESEGGLIETWNPNNKQFRCAGVALSRATLQRNALRRPYYSNAPQEIFIQQGNGYFGMVFPGCPETFEEPQESEQGEGRRYRDRHQKVNRFREGDIIAVPTGIVFWMYNDQDTPVIAVSLTDIRSSNNQLDQMPRRFYLAGNHEQEFLQYQHQQGGKQEQENEGNNIFSGFKRDYLEDAFNVNRHIVDRLQGRNEDEEKGAIVKVKGGLSIISPPEKQARHQRGSRQEEDEDEEKQPRHQRGSRQEEEEDEDEERQPRHQRRRGEEEEEDKKERGGSQKGKSRRQGDNGLEETVCTAKLRLNIGPSSSPDIYNPEAGRIKTVTSLDLPVLRWLKLSAEHGSLHKNAMFVPHYNLNANSIIYALKGRARLQVVNCNGNTVFDGELEAGRALTVPQNYAVAAKSLSDRFSYVAFKTNDRAGIARLAGTSSVINNLPLDVVAATFNLQRNEARQLKSNNPFKFLVPARESENRASA

SEQ ID NO: 355 - protein, 861 aa, Pisum sativum
MFSGVTGILNRGHKIKGTVVLMRKNVLDINSLTTVGGVIGQGFDILGSTVDNLTAFLGRSVSLQLISATKPDATGKGKLGKATFLEGIISSLPTLGAGQSAFKIHFEWDDDMGIPGAFYIKNFMQTEFFLVSLTLDDIPNHGSIYFVCNSWIYNAKHHKIDRIFFANQTYLPSETPAPLVHYREEELNNLRGDGTGERKEWERIYDYDVYNDLGNPDSGENHARPVLGGSETYPYPRRGRTGRKPTRKDPNSESRSDYVYLPRDEAFGHLKSSDFLTYGLKAVSQNVVPALESVFFDLNFTPNEFDSFDEVHGLYEGGIKLPTNILSQISPLPVLKEIFRTDGENTLKYPPPKVIQVSRSGWMTDEEFAREMLAGVNPNVICCLQEFPPRSKLDSQIYGDHTSKISKEHLEPNLEGLTVEEAIQNKKLFLLDHHDSIMPYLRRINSTSTKAYATRTILFLNNNQNLKPLAIELSLPHPQGDEHGAVSYVYQPALEGVESSIWLLAKAYVIVNDSCYHQLVSHWLNTHAVVEPFVIATNRHLSCLHPIYKLLYPHYRDTMNINSLARLSLVNDGGIIEKTFLWGRYSMEMSSKVYKNWVFTEQALPADLIKRGMAIEDPSSPCGVKLVVEDYPYAVDGLEIWAIIKTWVQDYVSLYYTSDEKLRQDSELQAWWKELVEVGHGDKKNEPWWPKMQTREDLIEVCSIVIWTASALHAAVNFGQYSYGGLILNRPTLSRRFMPEKGSAEFEELVKSPQKAYLKTITPKFQTLIDLSVIEILSRHASDELYLGERDNPNWTSDKRALEAFKKFGNKLAEIEKKLTQRNNDEKLRNRHGPVEMPYTLLYPSSKEGLTFRGIPNSISI

SEQ ID NO: 356 - protein, 10 aa, synthetic construct
  REGION 1..10: note = Synthetic peptide
KNPQLQDLDI

SEQ ID NO: 357 - protein, 11 aa, synthetic construct
  REGION 1..11: note = Synthetic peptide
GQSTSQWQSSR

SEQ ID NO: 358 - protein, 10 aa, synthetic construct
  REGION 1..10: note = Synthetic peptide
QSTSQWQSSR

SEQ ID NO: 359 - protein, 10 aa, synthetic construct
  REGION 1..10: note = Synthetic peptide
QSTSQWQSSR

SEQ ID NO: 360 - protein, 12 aa, synthetic construct
  REGION 1..12: note = Synthetic peptide
EEEEQGEEEINK

SEQ ID NO: 361 - protein, 10 aa, synthetic construct
  REGION 1..10: note = Synthetic peptide
PSTNPWHSPR

SEQ ID NO: 362 - protein, 14 aa, synthetic construct
  REGION 1..14: note = Synthetic peptide
AQAQDQYQQVQYSE

SEQ ID NO: 363 - protein, 26 aa, synthetic construct
  REGION 1..26: note = Synthetic peptide
SEAGVTEYFDEKNELFQCTGTFVIRR

SEQ ID NO: 364 - protein, 15 aa, synthetic construct
  REGION 1..15: note = Synthetic peptide
QAQAQDQYQQVQYSE

SEQ ID NO: 365 - protein, 13 aa, synthetic construct
  REGION 1..13: note = Synthetic peptide
GSMGLTFPGCPAT

SEQ ID NO: 366 - protein, 14 aa, synthetic construct
  REGION 1..14: note = Synthetic peptide
GSMGLTFPGCPATY

SEQ ID NO: 367 - protein, 8 aa, synthetic construct
  REGION 1..8: note = Synthetic peptide
LGAFTPRY

SEQ ID NO: 368 - protein, 10 aa, synthetic construct
  REGION 1..10: note = Synthetic peptide
LGAFTPRYQQ

SEQ ID NO: 369 - protein, 16 aa, synthetic construct
  REGION 1..16: note = Synthetic peptide
ALGVNALVAKRLQGQN

SEQ ID NO: 370 - protein, 9 aa, synthetic construct
  REGION 1..9: note = Synthetic peptide
LGAFTPRYQ

SEQ ID NO: 371 - protein, 13 aa, synthetic construct
  REGION 1..13: note = Synthetic peptide
GSMGLTFPGCPAT

SEQ ID NO: 372 - protein, 14 aa, synthetic construct
  REGION 1..14: note = Synthetic peptide
GSMGLTFPGCPATY

SEQ ID NO: 373 - protein, 14 aa, synthetic construct
  REGION 1..14: note = Synthetic peptide
SNNPFKFLVPARQS

SEQ ID NO: 374 - protein, 9 aa, synthetic construct
  REGION 1..9: note = Synthetic peptide
CAGVFVIRR

SEQ ID NO: 375 - protein, 10 aa, synthetic construct
  REGION 1..10: note = Synthetic peptide
GSPLQSPRGF

SEQ ID NO: 376 - protein, 9 aa, synthetic construct
  REGION 1..9: note = Synthetic peptide
RSSWQQQSY

SEQ ID NO: 377 - protein, 11 aa, synthetic construct
  REGION 1..11: note = Synthetic peptide
SFGGSPLQSPR

SEQ ID NO: 378 - protein, 11 aa, synthetic construct
  REGION 1..11: note = Synthetic peptide
YLPTKQLQPTW

SEQ ID NO: 379 - protein, 10 aa, synthetic construct
  REGION 1..10: note = Synthetic peptide
GKPRSSWQQQ

SEQ ID NO: 380 - protein, 11 aa, synthetic construct
  REGION 1..11: note = Synthetic peptide
FGGSPLQSPRG

SEQ ID NO: 381 - protein, 14 aa, synthetic construct
  REGION 1..14: note = Synthetic peptide
LNLLGFGINAENNE

SEQ ID NO: 382 - protein, 9 aa, synthetic construct
  REGION 1..9: note = Synthetic peptide
GALMLPHYN

SEQ ID NO: 383 - protein, 11 aa, synthetic construct
  REGION 1..11: note = Synthetic peptide
GALMLPHYNSR

SEQ ID NO: 384 - protein, 9 aa, synthetic construct
  REGION 1..9: note = Synthetic peptide
VFDGVLRPG

SEQ ID NO: 385 - protein, 6 aa, synthetic construct
  REGION 1..6: note = Synthetic peptide
LQSQND

SEQ ID NO: 386 - protein, 11 aa, synthetic construct
  REGION 1..11: note = Synthetic peptide
LQSQNDQRGEI

SEQ ID NO: 387 - protein, 14 aa, synthetic construct
  REGION 1..14: note = Synthetic peptide
QSQNDQRGEIIHVK

SEQ ID NO: 388 - protein, 8 aa, synthetic construct
  REGION 1..8: note = Synthetic peptide
RGEIIHVK

SEQ ID NO: 389 - protein, 8 aa, synthetic construct
  REGION 1..8: note = Synthetic peptide
RLQSQNDQ

SEQ ID NO: 390 - protein, 10 aa, synthetic construct
  REGION 1..10: note = Synthetic peptide
RLQSQNDQRG

SEQ ID NO: 391 - protein, 14 aa, synthetic construct
  REGION 1..14: note = Synthetic peptide
RLQSQNDQRGEIIH

SEQ ID NO: 392 - protein, 4 aa, synthetic construct
  REGION 1..4: note = Synthetic peptide
MPMP

SEQ ID NO: 393 - protein, 4 aa, synthetic construct
  REGION 1..4: note = Synthetic peptide
PMPL

SEQ ID NO: 394 - protein, 6 aa, synthetic construct
  REGION 1..6: note = Synthetic peptide
LEPDNR

SEQ ID NO: 395 - protein, 13 aa, synthetic construct
  REGION 1..13: note = Synthetic peptide
GIARLAGTSSVIN

SEQ ID NO: 396 - protein, 6 aa, synthetic construct
  REGION 1..6: note = Synthetic peptide
RSQNIF

SEQ ID NO: 397 - protein, 4 aa, synthetic construct
  REGION 1..4: note = Synthetic peptide
PNSM

SEQ ID NO: 398 - protein, 4 aa, synthetic construct
  REGION 1..4: note = Synthetic peptide
GHPM

SEQ ID NO: 399 - protein, 4 aa, synthetic construct
  REGION 1..4: note = Synthetic peptide
HPMS

SEQ ID NO: 400 - protein, 7 aa, synthetic construct
  REGION 1..7: note = Synthetic peptide
FLPQHTD

SEQ ID NO: 401 - protein, 7 aa, synthetic construct
  REGION 1..7: note = Synthetic peptide
EWQINEK

SEQ ID NO: 402 - protein, 13 aa, synthetic construct
  REGION 1..13: note = Synthetic peptide
GPQQYAEWQINEK

SEQ ID NO: 403 - protein, 8 aa, synthetic construct
  REGION 1..8: note = Synthetic peptide
PQQYAEWQ

SEQ ID NO: 404 - protein, 7 aa, synthetic construct
  REGION 1..7: note = Synthetic peptide
RGPQQYA

SEQ ID NO: 405 - protein, 4 aa, synthetic construct
  REGION 1..4: note = Synthetic peptide
HNPR

SEQ ID NO: 406-407 - skipped (2 entries)

SEQ ID NO: 408 - protein, 4 aa, synthetic construct
  REGION 1..4: note = Synthetic peptide
HPSF

SEQ ID NO: 409 - protein, 22 aa, synthetic construct
  REGION 1..22: note = Synthetic peptide
PGQLQSFLLSGNQNQQNYLSGF

SEQ ID NO: 410 - protein, 22 aa, synthetic construct
  REGION 1..22: note = Synthetic peptide
QLQSFLLSGNQNQQNYLSGFSK

SEQ ID NO: 411 - protein, 12 aa, synthetic construct
  REGION 1..12: note = Synthetic peptide
QSFLLSGNQNQQ

SEQ ID NO: 412 - protein, 12 aa, synthetic construct
  REGION 1..12: note = Synthetic peptide
PGQLQSFLLSGN

SEQ ID NO: 413 - protein, 9 aa, synthetic construct
  REGION 1..9: note = Synthetic peptide
QSFLLSGNQ

SEQ ID NO: 414 - protein, 12 aa, synthetic construct
  REGION 1..12: note = Synthetic peptide
QNQQNYLSGFSK

SEQ ID NO: 415 - protein, 6 aa, synthetic construct
  REGION 1..6: note = Synthetic peptide
YLRGFS

SEQ ID NO: 416 - protein, 11 aa, synthetic construct
  REGION 1..11: note = Synthetic peptide
PVEMPTLLYPS

SEQ ID NO: 417 - protein, 13 aa, synthetic construct
  REGION 1..13: note = Synthetic peptide
RGPQQYAEWQINE

SEQ ID NO: 418 - protein, 12 aa, synthetic construct
  REGION 1..12: note = Synthetic peptide
TVFDGVLRPGQL

SEQ ID NO: 419 - protein, 9 aa, synthetic construct
  REGION 1..9: note = Synthetic peptide
LDALEPDNR

SEQ ID NO: 420 - protein, 16 aa, synthetic construct
  REGION 1..16: note = Synthetic peptide
RLQSQNDQRGEIIHVK

SEQ ID NO: 421 - protein, 14 aa, synthetic construct
  REGION 1..14: note = Synthetic peptide
VLDLAIPVNRPGQL

SEQ ID NO: 422 - protein, 16 aa, synthetic construct
  REGION 1..16: note = Synthetic peptide
HGPVEMPYTLLYPSSK

SEQ ID NO: 423 - protein, 14 aa, synthetic construct
  REGION 1..14: note = Synthetic peptide
GYYGEQQQQPGMTR

SEQ ID NO: 424 - protein, 17 aa, synthetic construct
  REGION 1..17: note = Synthetic peptide
SEEGYYGEQQQQPGMTR

SEQ ID NO: 425 - protein, 6 aa, synthetic construct
  REGION 1..6: note = Synthetic peptide
LRGFSK